SEQUENCE LISTING (563 sequences)

SEQ ID NO: 1 - DNA, 609 bp, synthetic construct
  misc_feature 1..609: note = Synthetic
  source 1..609: mol_type = other DNA
cgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagaattcggcaagcggatggctgatgaaaccaagccaaccaggaagggcagcccacctatcaaggtgtactgccttccagacgaacgaagagagattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatga

SEQ ID NO: 2 - DNA, 609 bp, synthetic construct
  misc_feature 1..609: note = Synthetic
  source 1..609: mol_type = other DNA
cgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgagggcaagcggatggctgatgaaaccaagccaaccaggaagggcagcccacctatcaaggtgtactgccttccagacgaacgaagagagattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatga

SEQ ID NO: 3 - skipped

SEQ ID NO: 4 - DNA, 19 bp, synthetic construct
  misc_feature 1..19: note = Synthetic
  source 1..19: mol_type = other DNA
aggcggtaaaggtgagcag

SEQ ID NO: 5 - DNA, 19 bp, synthetic construct
  misc_feature 1..19: note = Synthetic
  source 1..19: mol_type = other DNA
gaagcctgcgaagagttgc

SEQ ID NO: 6 - DNA, 20 bp, synthetic construct
  misc_feature 1..20: note = Synthetic
  source 1..20: mol_type = other DNA
ctctgtttatcggcagttcg

SEQ ID NO: 7 - DNA, 20 bp, synthetic construct
  misc_feature 1..20: note = Synthetic
  source 1..20: mol_type = other DNA
gtattcgtgcagggcaagat

SEQ ID NO: 8 - DNA, 20 bp, synthetic construct
  misc_feature 1..20: note = Synthetic
  source 1..20: mol_type = other DNA
ctctgtttatcggcagttcg

SEQ ID NO: 9 - DNA, 20 bp, synthetic construct
  misc_feature 1..20: note = Synthetic
  source 1..20: mol_type = other DNA
tcgatggtccagcaagctac

SEQ ID NO: 10 - DNA, 20 bp, synthetic construct
  misc_feature 1..20: note = Synthetic
  source 1..20: mol_type = other DNA
ctcggtgtcacgggtaagat

SEQ ID NO: 11 - DNA, 20 bp, synthetic construct
  misc_feature 1..20: note = Synthetic
  source 1..20: mol_type = other DNA
cacgacgtaggggttctgat

SEQ ID NO: 12 - DNA, 20 bp, synthetic construct
  misc_feature 1..20: note = Synthetic
  source 1..20: mol_type = other DNA
tgcgctttgacagttgtttt

SEQ ID NO: 13 - DNA, 20 bp, synthetic construct
  misc_feature 1..20: note = Synthetic
  source 1..20: mol_type = other DNA
ttcgtcagccagctctcata

SEQ ID NO: 14 - DNA, 20 bp, synthetic construct
  misc_feature 1..20: note = Synthetic
  source 1..20: mol_type = other DNA
attcaccgaaacccattgag

SEQ ID NO: 15 - DNA, 20 bp, synthetic construct
  misc_feature 1..20: note = Synthetic
  source 1..20: mol_type = other DNA
catgattcggaacgcataga

SEQ ID NO: 16 - DNA, 20 bp, synthetic construct
  misc_feature 1..20: note = Synthetic
  source 1..20: mol_type = other DNA
ttcttccgaaccctgatctt

SEQ ID NO: 17 - DNA, 20 bp, synthetic construct
  misc_feature 1..20: note = Synthetic
  source 1..20: mol_type = other DNA
tccttggaacgatgcttttt

SEQ ID NO: 18 - DNA, 20 bp, synthetic construct
  misc_feature 1..20: note = Synthetic
  source 1..20: mol_type = other DNA
gtcggtctgatcctgtggtt

SEQ ID NO: 19 - DNA, 20 bp, synthetic construct
  misc_feature 1..20: note = Synthetic
  source 1..20: mol_type = other DNA
gtctgcttttgcgacacaac

SEQ ID NO: 20 - DNA, 20 bp, synthetic construct
  misc_feature 1..20: note = Synthetic
  source 1..20: mol_type = other DNA
cgtctctacgacctggcact

SEQ ID NO: 21 - DNA, 19 bp, synthetic construct
  misc_feature 1..19: note = Synthetic
  source 1..19: mol_type = other DNA
gtacccgcctcccacttct

SEQ ID NO: 22 - DNA, 20 bp, synthetic construct
  misc_feature 1..20: note = Synthetic
  source 1..20: mol_type = other DNA
ggtcagcaccgaaatcactt

SEQ ID NO: 23 - DNA, 19 bp, synthetic construct
  misc_feature 1..19: note = Synthetic
  source 1..19: mol_type = other DNA
agctgccgtacttggaggt

SEQ ID NO: 24 - DNA, 20 bp, synthetic construct
  misc_feature 1..20: note = Synthetic
  source 1..20: mol_type = other DNA
acggcaagaaaaccatcatc

SEQ ID NO: 25 - DNA, 20 bp, synthetic construct
  misc_feature 1..20: note = Synthetic
  source 1..20: mol_type = other DNA
tgaggtacatcggcatgttg

SEQ ID NO: 26 - DNA, 20 bp, synthetic construct
  misc_feature 1..20: note = Synthetic
  source 1..20: mol_type = other DNA
agcaacctgtatcgcctgac

SEQ ID NO: 27 - DNA, 20 bp, synthetic construct
  misc_feature 1..20: note = Synthetic
  source 1..20: mol_type = other DNA
tcctcgatttgcttctggac

SEQ ID NO: 28 - DNA, 20 bp, synthetic construct
  misc_feature 1..20: note = Synthetic
  source 1..20: mol_type = other DNA
gcagatgatcgccaaaaact

SEQ ID NO: 29 - DNA, 20 bp, synthetic construct
  misc_feature 1..20: note = Synthetic
  source 1..20: mol_type = other DNA
gggcacttcttcaacagctc

SEQ ID NO: 30 - DNA, 18 bp, synthetic construct
  misc_feature 1..18: note = Synthetic
  source 1..18: mol_type = other DNA
caaatccgtggccttctg

SEQ ID NO: 31 - DNA, 20 bp, synthetic construct
  misc_feature 1..20: note = Synthetic
  source 1..20: mol_type = other DNA
acggtcaaggtccagaacag

SEQ ID NO: 32 - DNA, 18 bp, synthetic construct
  misc_feature 1..18: note = Synthetic
  source 1..18: mol_type = other DNA
gatctgccgaaggtcacg

SEQ ID NO: 33 - DNA, 20 bp, synthetic construct
  misc_feature 1..20: note = Synthetic
  source 1..20: mol_type = other DNA
cttgtggatgccgaagtacc

SEQ ID NO: 34 - DNA, 20 bp, synthetic construct
  misc_feature 1..20: note = Synthetic
  source 1..20: mol_type = other DNA
gttcgacgacaacagccttt

SEQ ID NO: 35 - DNA, 20 bp, synthetic construct
  misc_feature 1..20: note = Synthetic
  source 1..20: mol_type = other DNA
aagaatgttggccggaatct

SEQ ID NO: 36 - DNA, 20 bp, synthetic construct
  misc_feature 1..20: note = Synthetic
  source 1..20: mol_type = other DNA
gccatgcctataccgacttc

SEQ ID NO: 37 - DNA, 20 bp, synthetic construct
  misc_feature 1..20: note = Synthetic
  source 1..20: mol_type = other DNA
tgcccagtaccagaagatca

SEQ ID NO: 38 - DNA, 20 bp, synthetic construct
  misc_feature 1..20: note = Synthetic
  source 1..20: mol_type = other DNA
ggcagctcatcatcaacaac

SEQ ID NO: 39 - DNA, 20 bp, synthetic construct
  misc_feature 1..20: note = Synthetic
  source 1..20: mol_type = other DNA
ccaggacgaaaacgaaaaga

SEQ ID NO: 40 - DNA, 19 bp, synthetic construct
  misc_feature 1..19: note = Synthetic
  source 1..19: mol_type = other DNA
aagcgccgtttgatcttct

SEQ ID NO: 41 - DNA, 19 bp, synthetic construct
  misc_feature 1..19: note = Synthetic
  source 1..19: mol_type = other DNA
catggggatgttcagcagt

SEQ ID NO: 42 - DNA, 20 bp, synthetic construct
  misc_feature 1..20: note = Synthetic
  source 1..20: mol_type = other DNA
acgtactacccgctgcatct

SEQ ID NO: 43 - DNA, 20 bp, synthetic construct
  misc_feature 1..20: note = Synthetic
  source 1..20: mol_type = other DNA
gatagaacgcttcggtgtcc

SEQ ID NO: 44 - DNA, 20 bp, synthetic construct
  misc_feature 1..20: note = Synthetic
  source 1..20: mol_type = other DNA
ataaccagctcgccatcaag

SEQ ID NO: 45 - DNA, 19 bp, synthetic construct
  misc_feature 1..19: note = Synthetic
  source 1..19: mol_type = other DNA
cagcaccaggaacatcgtc

SEQ ID NO: 46 - DNA, 20 bp, synthetic construct
  misc_feature 1..20: note = Synthetic
  source 1..20: mol_type = other DNA
cgagctgctgaacaaggttt

SEQ ID NO: 47 - DNA, 20 bp, synthetic construct
  misc_feature 1..20: note = Synthetic
  source 1..20: mol_type = other DNA
gagccaggtcaaacgagtgt

SEQ ID NO: 48 - DNA, 20 bp, synthetic construct
  misc_feature 1..20: note = Synthetic
  source 1..20: mol_type = other DNA
gactacaccgagggggaaag

SEQ ID NO: 49 - DNA, 20 bp, synthetic construct
  misc_feature 1..20: note = Synthetic
  source 1..20: mol_type = other DNA
gagaaggacaccgaccgtta

SEQ ID NO: 50 - DNA, 20 bp, synthetic construct
  misc_feature 1..20: note = Synthetic
  source 1..20: mol_type = other DNA
gactacaccgagggggaaag

SEQ ID NO: 51 - DNA, 19 bp, synthetic construct
  misc_feature 1..19: note = Synthetic
  source 1..19: mol_type = other DNA
gtgggtcaacatggagctg

SEQ ID NO: 52 - DNA, 20 bp, synthetic construct
  misc_feature 1..20: note = Synthetic
  source 1..20: mol_type = other DNA
tctcctggcgttcaagattc

SEQ ID NO: 53 - DNA, 20 bp, synthetic construct
  misc_feature 1..20: note = Synthetic
  source 1..20: mol_type = other DNA
tccgtcatgtcgattgtcag

SEQ ID NO: 54 - DNA, 20 bp, synthetic construct
  misc_feature 1..20: note = Synthetic
  source 1..20: mol_type = other DNA
tgcttgtctaccagcacgtc

SEQ ID NO: 55 - DNA, 20 bp, synthetic construct
  misc_feature 1..20: note = Synthetic
  source 1..20: mol_type = other DNA
tccgtcatgtcgattgtcag

SEQ ID NO: 56 - DNA, 20 bp, synthetic construct
  misc_feature 1..20: note = Synthetic
  source 1..20: mol_type = other DNA
catggcgcattacagcaata

SEQ ID NO: 57 - DNA, 20 bp, synthetic construct
  misc_feature 1..20: note = Synthetic
  source 1..20: mol_type = other DNA
gggaaagagttcgctcatgt

SEQ ID NO: 58 - DNA, 20 bp, synthetic construct
  misc_feature 1..20: note = Synthetic
  source 1..20: mol_type = other DNA
cgaggttggtggtaatcgtt

SEQ ID NO: 59 - DNA, 20 bp, synthetic construct
  misc_feature 1..20: note = Synthetic
  source 1..20: mol_type = other DNA
gccgcatagtgtagccagat

SEQ ID NO: 60 - DNA, 20 bp, synthetic construct
  misc_feature 1..20: note = Synthetic
  source 1..20: mol_type = other DNA
agactagccaccaccattgc

SEQ ID NO: 61 - DNA, 20 bp, synthetic construct
  misc_feature 1..20: note = Synthetic
  source 1..20: mol_type = other DNA
acggaggtagcagcagaaaa

SEQ ID NO: 62 - DNA, 20 bp, synthetic construct
  misc_feature 1..20: note = Synthetic
  source 1..20: mol_type = other DNA
tgcaatttcatctcctgctg

SEQ ID NO: 63 - DNA, 20 bp, synthetic construct
  misc_feature 1..20: note = Synthetic
  source 1..20: mol_type = other DNA
cgcaacgtctaccagttcag

SEQ ID NO: 64 - DNA, 20 bp, synthetic construct
  misc_feature 1..20: note = Synthetic
  source 1..20: mol_type = other DNA
gttgacgatgatgctgttgg

SEQ ID NO: 65 - DNA, 20 bp, synthetic construct
  misc_feature 1..20: note = Synthetic
  source 1..20: mol_type = other DNA
gccaggtgagaagtgctgtc

SEQ ID NO: 66 - DNA, 20 bp, synthetic construct
  misc_feature 1..20: note = Synthetic
  source 1..20: mol_type = other DNA
accttcatggtcagccattc

SEQ ID NO: 67 - DNA, 19 bp, synthetic construct
  misc_feature 1..19: note = Synthetic
  source 1..19: mol_type = other DNA
gagttcgacccggaagaag

SEQ ID NO: 68 - DNA, 20 bp, synthetic construct
  misc_feature 1..20: note = Synthetic
  source 1..20: mol_type = other DNA
accttcatggtcagccattc

SEQ ID NO: 69 - DNA, 20 bp, synthetic construct
  misc_feature 1..20: note = Synthetic
  source 1..20: mol_type = other DNA
atcaaggcgctacaagagga

SEQ ID NO: 70 - DNA, 20 bp, synthetic construct
  misc_feature 1..20: note = Synthetic
  source 1..20: mol_type = other DNA
tgtaacgcttcccggtagtc

SEQ ID NO: 71 - DNA, 20 bp, synthetic construct
  misc_feature 1..20: note = Synthetic
  source 1..20: mol_type = other DNA
agtctccgagctgcacaagt

SEQ ID NO: 72 - DNA, 20 bp, synthetic construct
  misc_feature 1..20: note = Synthetic
  source 1..20: mol_type = other DNA
accaggtcaatgtcgctctc

SEQ ID NO: 73 - DNA, 20 bp, synthetic construct
  misc_feature 1..20: note = Synthetic
  source 1..20: mol_type = other DNA
gccgaggttctgcttatgtc

SEQ ID NO: 74 - DNA, 20 bp, synthetic construct
  misc_feature 1..20: note = Synthetic
  source 1..20: mol_type = other DNA
tagtcctccgggtctagcaa

SEQ ID NO: 75 - DNA, 20 bp, synthetic construct
  misc_feature 1..20: note = Synthetic
  source 1..20: mol_type = other DNA
gcgacatggttgtgtacgtc

SEQ ID NO: 76 - DNA, 20 bp, synthetic construct
  misc_feature 1..20: note = Synthetic
  source 1..20: mol_type = other DNA
gcttttggtggtgttgacct

SEQ ID NO: 77 - DNA, 20 bp, synthetic construct
  misc_feature 1..20: note = Synthetic
  source 1..20: mol_type = other DNA
tgattatgttggtgcgctgt

SEQ ID NO: 78 - DNA, 19 bp, synthetic construct
  misc_feature 1..19: note = Synthetic
  source 1..19: mol_type = other DNA
tttttcgcccgtatctgtg

SEQ ID NO: 79 - DNA, 20 bp, synthetic construct
  misc_feature 1..20: note = Synthetic
  source 1..20: mol_type = other DNA
gtatccaacggcgtcagaat

SEQ ID NO: 80 - DNA, 20 bp, synthetic construct
  misc_feature 1..20: note = Synthetic
  source 1..20: mol_type = other DNA
gcaccagaatctcgtcgttt

SEQ ID NO: 81 - DNA, 19 bp, synthetic construct
  misc_feature 1..19: note = Synthetic
  source 1..19: mol_type = other DNA
gtcaacggcacagcagagt

SEQ ID NO: 82 - DNA, 18 bp, synthetic construct
  misc_feature 1..18: note = Synthetic
  source 1..18: mol_type = other DNA
ctcggtcttgccttgctc

SEQ ID NO: 83 - DNA, 20 bp, synthetic construct
  misc_feature 1..20: note = Synthetic
  source 1..20: mol_type = other DNA
gtatccaacggcgtcagaat

SEQ ID NO: 84 - DNA, 20 bp, synthetic construct
  misc_feature 1..20: note = Synthetic
  source 1..20: mol_type = other DNA
gcgaagtcgctcttcttgat

SEQ ID NO: 85 - DNA, 20 bp, synthetic construct
  misc_feature 1..20: note = Synthetic
  source 1..20: mol_type = other DNA
gctctttggcatcgtctctc

SEQ ID NO: 86 - DNA, 20 bp, synthetic construct
  misc_feature 1..20: note = Synthetic
  source 1..20: mol_type = other DNA
tcgctataatgaccccgaag

SEQ ID NO: 87 - DNA, 20 bp, synthetic construct
  misc_feature 1..20: note = Synthetic
  source 1..20: mol_type = other DNA
gttgcgcgagttaatttcgt

SEQ ID NO: 88 - DNA, 20 bp, synthetic construct
  misc_feature 1..20: note = Synthetic
  source 1..20: mol_type = other DNA
aacggggaaggtcaagttct

SEQ ID NO: 89 - DNA, 20 bp, synthetic construct
  misc_feature 1..20: note = Synthetic
  source 1..20: mol_type = other DNA
tgatggtatgcaggatcagc

SEQ ID NO: 90 - DNA, 20 bp, synthetic construct
  misc_feature 1..20: note = Synthetic
  source 1..20: mol_type = other DNA
ggcaagagctttgagcagat

SEQ ID NO: 91 - DNA, 20 bp, synthetic construct
  misc_feature 1..20: note = Synthetic
  source 1..20: mol_type = other DNA
tgtcaaacaagcccagctaa

SEQ ID NO: 92 - DNA, 20 bp, synthetic construct
  misc_feature 1..20: note = Synthetic
  source 1..20: mol_type = other DNA
ggcgaaggtaatgaaggaca

SEQ ID NO: 93 - DNA, 20 bp, synthetic construct
  misc_feature 1..20: note = Synthetic
  source 1..20: mol_type = other DNA
tgcgaaaagcatcacctatg

SEQ ID NO: 94 - DNA, 20 bp, synthetic construct
  misc_feature 1..20: note = Synthetic
  source 1..20: mol_type = other DNA
tggttctcggtctggacaat

SEQ ID NO: 95 - DNA, 20 bp, synthetic construct
  misc_feature 1..20: note = Synthetic
  source 1..20: mol_type = other DNA
acctgccgttaagtcgagaa

SEQ ID NO: 96 - DNA, 21 bp, synthetic construct
  misc_feature 1..21: note = Synthetic
  source 1..21: mol_type = other DNA
ggatagcttgcaacatcagga

SEQ ID NO: 97 - DNA, 20 bp, synthetic construct
  misc_feature 1..20: note = Synthetic
  source 1..20: mol_type = other DNA
acccggacaagctgaaaaag

SEQ ID NO: 98 - DNA, 20 bp, synthetic construct
  misc_feature 1..20: note = Synthetic
  source 1..20: mol_type = other DNA
ttggttgaacatagcggtga

SEQ ID NO: 99 - DNA, 20 bp, synthetic construct
  misc_feature 1..20: note = Synthetic
  source 1..20: mol_type = other DNA
gttcagacgacgctcatcaa

SEQ ID NO: 100 - DNA, 19 bp, synthetic construct
  misc_feature 1..19: note = Synthetic
  source 1..19: mol_type = other DNA
ctggtcgctgaatgtcgat

SEQ ID NO: 101 - DNA, 20 bp, synthetic construct
  misc_feature 1..20: note = Synthetic
  source 1..20: mol_type = other DNA
gctcatcaccaccaagaaca

SEQ ID NO: 102 - DNA, 20 bp, synthetic construct
  misc_feature 1..20: note = Synthetic
  source 1..20: mol_type = other DNA
catgtcgaaggcgacgatag

SEQ ID NO: 103 - DNA, 20 bp, synthetic construct
  misc_feature 1..20: note = Synthetic
  source 1..20: mol_type = other DNA
ttcgatggatgtttgctttg

SEQ ID NO: 104 - DNA, 20 bp, synthetic construct
  misc_feature 1..20: note = Synthetic
  source 1..20: mol_type = other DNA
gacagctcgttgagggaatc

SEQ ID NO: 105 - DNA, 20 bp, synthetic construct
  misc_feature 1..20: note = Synthetic
  source 1..20: mol_type = other DNA
ggtactgaccgcactcacct

SEQ ID NO: 106 - DNA, 20 bp, synthetic construct
  misc_feature 1..20: note = Synthetic
  source 1..20: mol_type = other DNA
caaaacctcccccttcaatc

SEQ ID NO: 107 - DNA, 20 bp, synthetic construct
  misc_feature 1..20: note = Synthetic
  source 1..20: mol_type = other DNA
tttgagcaatgccaagacag

SEQ ID NO: 108 - DNA, 20 bp, synthetic construct
  misc_feature 1..20: note = Synthetic
  source 1..20: mol_type = other DNA
ctccggattgaaccagtacg

SEQ ID NO: 109 - DNA, 20 bp, synthetic construct
  misc_feature 1..20: note = Synthetic
  source 1..20: mol_type = other DNA
aagtcaccaagtgggtcgag

SEQ ID NO: 110 - DNA, 20 bp, synthetic construct
  misc_feature 1..20: note = Synthetic
  source 1..20: mol_type = other DNA
cacgcctgggaacttgataa

SEQ ID NO: 111 - DNA, 20 bp, synthetic construct
  misc_feature 1..20: note = Synthetic
  source 1..20: mol_type = other DNA
gaatgctattgccgagaagc

SEQ ID NO: 112 - DNA, 20 bp, synthetic construct
  misc_feature 1..20: note = Synthetic
  source 1..20: mol_type = other DNA
ctcgacccacttggtgactt

SEQ ID NO: 113 - DNA, 20 bp, synthetic construct
  misc_feature 1..20: note = Synthetic
  source 1..20: mol_type = other DNA
caggggaaaggtgttttcaa

SEQ ID NO: 114 - DNA, 20 bp, synthetic construct
  misc_feature 1..20: note = Synthetic
  source 1..20: mol_type = other DNA
gcttctcggcaatagcattc

SEQ ID NO: 115 - DNA, 20 bp, synthetic construct
  misc_feature 1..20: note = Synthetic
  source 1..20: mol_type = other DNA
actggtccacccaggaagtc

SEQ ID NO: 116 - DNA, 19 bp, synthetic construct
  misc_feature 1..19: note = Synthetic
  source 1..19: mol_type = other DNA
ggctaagggtcgaaatgga

SEQ ID NO: 117 - DNA, 20 bp, synthetic construct
  misc_feature 1..20: note = Synthetic
  source 1..20: mol_type = other DNA
actttacgccaagggagagg

SEQ ID NO: 118 - DNA, 60 bp, synthetic construct
  misc_feature 1..60: note = Synthetic
  source 1..60: mol_type = other DNA
gcggcagagatgaacacgaccatcagcggctgcacagcgccattgacccaggcgtgttcc

SEQ ID NO: 119 - DNA, 59 bp, synthetic construct
  misc_feature 1..59: note = Synthetic
  source 1..59: mol_type = other DNA
tgcgaggcagcggcctggtggaacacgcctgggtcaatggcgctgtgcagccgctgatg

SEQ ID NO: 120 - DNA, 60 bp, synthetic construct
  misc_feature 1..60: note = Synthetic
  source 1..60: mol_type = other DNA
tggctgctgaacccccagccggaactgaccccacaaggcctcaccctccttgcgggattg

SEQ ID NO: 121 - DNA, 60 bp, synthetic construct
  misc_feature 1..60: note = Synthetic
  source 1..60: mol_type = other DNA
tgccccggcgtgagtcggggcaatcccgcaaggagggtgaggccttgtggggtcagttcc

SEQ ID NO: 122 - DNA, 60 bp, synthetic construct
  misc_feature 1..60: note = Synthetic
  source 1..60: mol_type = other DNA
tggctgctgaacccccagccggaactgaccccacaaggccagttcctcgcgtgtcgatgg

SEQ ID NO: 123 - DNA, 60 bp, synthetic construct
  misc_feature 1..60: note = Synthetic
  source 1..60: mol_type = other DNA
aggtttggcgaagtcgatgaccatcgacacgcgaggaactggccttgtggggtcagttcc

SEQ ID NO: 124 - DNA, 60 bp, synthetic construct
  misc_feature 1..60: note = Synthetic
  source 1..60: mol_type = other DNA
atttttcaccaacatccttcgtctgctcgatgagcggggccgccaagggttagggcttgc

SEQ ID NO: 125 - DNA, 60 bp, synthetic construct
  misc_feature 1..60: note = Synthetic
  source 1..60: mol_type = other DNA
tagcggctaaagaaggaagtgcaagccctaacccttggcggccccgctcatcgagcagac

SEQ ID NO: 126 - DNA, 60 bp, synthetic construct
  misc_feature 1..60: note = Synthetic
  source 1..60: mol_type = other DNA
gaccatcaaggagcgggccaagcgcaagctggaacgcgacggagtatttccaatgacaac

SEQ ID NO: 127 - DNA, 60 bp, synthetic construct
  misc_feature 1..60: note = Synthetic
  source 1..60: mol_type = other DNA
tcagacggaacggaacagccgttgtcattggaaatactccgtcgcgttccagcttgcgct

SEQ ID NO: 128 - DNA, 60 bp, synthetic construct
  misc_feature 1..60: note = Synthetic
  source 1..60: mol_type = other DNA
cggccagtacatcaccaaaaccctgtaaggagtatttccatcatggctctgcgcacgatc

SEQ ID NO: 129 - DNA, 60 bp, synthetic construct
  misc_feature 1..60: note = Synthetic
  source 1..60: mol_type = other DNA
ttgcctgcgcgacggatggggatcgtgcgcagagccatgatggaaatactccttacaggg

SEQ ID NO: 130 - DNA, 60 bp, synthetic construct
  misc_feature 1..60: note = Synthetic
  source 1..60: mol_type = other DNA
atgccgtgcgtgcggtagcggctggacggctcgcctaatcatgatccaagcaattgcgat

SEQ ID NO: 131 - DNA, 60 bp, synthetic construct
  misc_feature 1..60: note = Synthetic
  source 1..60: mol_type = other DNA
cgccgaggcccgcgattgcaatcgcaattgcttggatcatgattaggcgagccgtccagc

SEQ ID NO: 132 - DNA, 60 bp, synthetic construct
  misc_feature 1..60: note = Synthetic
  source 1..60: mol_type = other DNA
ccgcgagaacaccaatagccaagggaagcaataccgatgaatgagttttgcagacacgat

SEQ ID NO: 133 - DNA, 60 bp, synthetic construct
  misc_feature 1..60: note = Synthetic
  source 1..60: mol_type = other DNA
tcttgaagatcaagcccttgatcgtgtctgcaaaactcattcatcggtattgcttccctt

SEQ ID NO: 134 - DNA, 60 bp, synthetic construct
  misc_feature 1..60: note = Synthetic
  source 1..60: mol_type = other DNA
ccggggcctcgcccttgatgaatacctggaggcagcatgaggcactgaattatgaaaaag

SEQ ID NO: 135 - DNA, 60 bp, synthetic construct
  misc_feature 1..60: note = Synthetic
  source 1..60: mol_type = other DNA
aggaccaaagcaaacagttcctttttcataattcagtgcctcatgctgcctccaggtatt

SEQ ID NO: 136 - DNA, 60 bp, synthetic construct
  misc_feature 1..60: note = Synthetic
  source 1..60: mol_type = other DNA
gggacttctcctggtcgagacttctgtgaggcactgaattaccatgcgtaagattctgac

SEQ ID NO: 137 - DNA, 60 bp, synthetic construct
  misc_feature 1..60: note = Synthetic
  source 1..60: mol_type = other DNA
tggccgcgagtgcgatgacggtcagaatcttacgcatggtaattcagtgcctcacagaag

SEQ ID NO: 138 - DNA, 60 bp, synthetic construct
  misc_feature 1..60: note = Synthetic
  source 1..60: mol_type = other DNA
cagccaggaccgcgtgaccatttcaagggggaactaaaccgccaatgagcgaagatcaaa

SEQ ID NO: 139 - DNA, 60 bp, synthetic construct
  misc_feature 1..60: note = Synthetic
  source 1..60: mol_type = other DNA
tggcgatgcgtccggtgccatttgatcttcgctcattggcggtttagttcccccttgaaa

SEQ ID NO: 140 - DNA, 60 bp, synthetic construct
  misc_feature 1..60: note = Synthetic
  source 1..60: mol_type = other DNA
atggtcccggccggcgcatgggttcggaaggagtaagccactccaaggagtaacttatga

SEQ ID NO: 141 - DNA, 60 bp, synthetic construct
  misc_feature 1..60: note = Synthetic
  source 1..60: mol_type = other DNA
aacattcttagcgagcttcttcataagttactccttggagtggcttactccttccgaacc

SEQ ID NO: 142 - DNA, 60 bp, synthetic construct
  misc_feature 1..60: note = Synthetic
  source 1..60: mol_type = other DNA
ccctaccaggcgtttgactattaactccaaggagtaacttggggaggcgcgatgaagaaa

SEQ ID NO: 143 - DNA, 60 bp, synthetic construct
  misc_feature 1..60: note = Synthetic
  source 1..60: mol_type = other DNA
gcaactgcgatgaagttggatttcttcatcgcgcctccccaagttactccttggagttaa

SEQ ID NO: 144 - DNA, 60 bp, synthetic construct
  misc_feature 1..60: note = Synthetic
  source 1..60: mol_type = other DNA
accgcgcaagcccgtctaagacctggtgaggggaggcgcgtgacgtatgaaaatccagac

SEQ ID NO: 145 - DNA, 60 bp, synthetic construct
  misc_feature 1..60: note = Synthetic
  source 1..60: mol_type = other DNA
ccgcgagcgcggcagctctagtctggattttcatacgtcacgcgcctcccctcaccaggt

SEQ ID NO: 146 - DNA, 60 bp, synthetic construct
  misc_feature 1..60: note = Synthetic
  source 1..60: mol_type = other DNA
aggcttcaagcccagcgaaaagaaagagtggtgatgacgtaacgactcttaggagctacg

SEQ ID NO: 147 - DNA, 60 bp, synthetic construct
  misc_feature 1..60: note = Synthetic
  source 1..60: mol_type = other DNA
gcttttttcagttgcatggtcgtagctcctaagagtcgttacgtcatcaccactctttct

SEQ ID NO: 148 - DNA, 60 bp, synthetic construct
  misc_feature 1..60: note = Synthetic
  source 1..60: mol_type = other DNA
agcccgcccaatcctgaaacgactcttaggagctacgaccggggaggggatagcgatgcc

SEQ ID NO: 149 - DNA, 60 bp, synthetic construct
  misc_feature 1..60: note = Synthetic
  source 1..60: mol_type = other DNA
gtgccagcagcttggcaaacggcatcgctatcccctccccggtcgtagctcctaagagtc

SEQ ID NO: 150 - DNA, 60 bp, synthetic construct
  misc_feature 1..60: note = Synthetic
  source 1..60: mol_type = other DNA
cgcctaccgtcggttcgagcggtaaggggaggggatagcgggaggaacggccgtttagcg

SEQ ID NO: 151 - DNA, 60 bp, synthetic construct
  misc_feature 1..60: note = Synthetic
  source 1..60: mol_type = other DNA
gaatgcccataggctttagccgctaaacggccgttcctcccgctatcccctccccttacc

SEQ ID NO: 152 - DNA, 60 bp, synthetic construct
  misc_feature 1..60: note = Synthetic
  source 1..60: mol_type = other DNA
cacgccataaggaggaacggccgtttagcggctaaagcctcgccctgcagggcgttctta

SEQ ID NO: 153 - DNA, 60 bp, synthetic construct
  misc_feature 1..60: note = Synthetic
  source 1..60: mol_type = other DNA
tcatgccctcccccttggagtaagaacgccctgcagggcgaggctttagccgctaaacgg

SEQ ID NO: 154 - DNA, 60 bp, synthetic construct
  misc_feature 1..60: note = Synthetic
  source 1..60: mol_type = other DNA
atagcgccctgcagggcgttcttactccaagggggagggccctttgagattccaatatgc

SEQ ID NO: 155 - DNA, 60 bp, synthetic construct
  misc_feature 1..60: note = Synthetic
  source 1..60: mol_type = other DNA
ggtgcatttcttgagcaattgcatattggaatctcaaagggccctcccccttggagtaag

SEQ ID NO: 156 - DNA, 60 bp, synthetic construct
  misc_feature 1..60: note = Synthetic
  source 1..60: mol_type = other DNA
gcgggctacttgtttttcacctgacctttgagattccaatggggagggcggcggatgctg

SEQ ID NO: 157 - DNA, 60 bp, synthetic construct
  misc_feature 1..60: note = Synthetic
  source 1..60: mol_type = other DNA
aggaagcccttcaaccgtgtcagcatccgccgccctccccattggaatctcaaaggtcag

SEQ ID NO: 158 - DNA, 60 bp, synthetic construct
  misc_feature 1..60: note = Synthetic
  source 1..60: mol_type = other DNA
cgcttgacgatgcgaagttctactgaggggagggcggcggctacaaccgtgcgcaaggcg

SEQ ID NO: 159 - DNA, 60 bp, synthetic construct
  misc_feature 1..60: note = Synthetic
  source 1..60: mol_type = other DNA
tacatacatcctccctaatgcgccttgcgcacggttgtagccgccgccctcccctcagta

SEQ ID NO: 160 - DNA, 60 bp, synthetic construct
  misc_feature 1..60: note = Synthetic
  source 1..60: mol_type = other DNA
tctgactacaaccgtgcgcaaggcgcattagggaggatgtgaaaagccgggcactgcccg

SEQ ID NO: 161 - DNA, 60 bp, synthetic construct
  misc_feature 1..60: note = Synthetic
  source 1..60: mol_type = other DNA
cgcagcagcaaaaataaagccgggcagtgcccggcttttcacatcctccctaatgcgcct

SEQ ID NO: 162 - DNA, 60 bp, synthetic construct
  misc_feature 1..60: note = Synthetic
  source 1..60: mol_type = other DNA
gggacgcgaaaaggtgagaaaagccgggcactgcccggctttccacggctcgacggcgtg

SEQ ID NO: 163 - DNA, 60 bp, synthetic construct
  misc_feature 1..60: note = Synthetic
  source 1..60: mol_type = other DNA
tcggcctctggtccgatccgcacgccgtcgagccgtggaaagccgggcagtgcccggctt

SEQ ID NO: 164 - DNA, 60 bp, synthetic construct
  misc_feature 1..60: note = Synthetic
  source 1..60: mol_type = other DNA
gggacgcgaaaaggtgagaaaagccgggcactgcccggctccttcgtccctccggttgtt

SEQ ID NO: 165 - DNA, 60 bp, synthetic construct
  misc_feature 1..60: note = Synthetic
  source 1..60: mol_type = other DNA
ggaaatggcgacgcgagagcaacaaccggagggacgaaggagccgggcagtgcccggctt

SEQ ID NO: 166 - DNA, 60 bp, synthetic construct
  misc_feature 1..60: note = Synthetic
  source 1..60: mol_type = other DNA
tgctcgtccgtactggcgcgcaggtagatgcgggcgacctggcgcattacagcaatacgc

SEQ ID NO: 167 - DNA, 60 bp, synthetic construct
  misc_feature 1..60: note = Synthetic
  source 1..60: mol_type = other DNA
gcgctaggcgcatttaaattgcgtattgctgtaatgcgccaggtcgcccgcatctacctg

SEQ ID NO: 168 - DNA, 60 bp, synthetic construct
  misc_feature 1..60: note = Synthetic
  source 1..60: mol_type = other DNA
agcgcggcagcggcgcgcagcatggcgtagcttcggcgctttgggcctagtctagccggc

SEQ ID NO: 169 - DNA, 60 bp, synthetic construct
  misc_feature 1..60: note = Synthetic
  source 1..60: mol_type = other DNA
gtattgctgtaatgcgccatgccggctagactaggcccaaagcgccgaagctacgccatg

SEQ ID NO: 170 - DNA, 60 bp, synthetic construct
  misc_feature 1..60: note = Synthetic
  source 1..60: mol_type = other DNA
tgtgctaatgtggttacgtgtattttatggaggttatccacggcctacatcctcacggct

SEQ ID NO: 171 - DNA, 60 bp, synthetic construct
  misc_feature 1..60: note = Synthetic
  source 1..60: mol_type = other DNA
cgtagatcggcttcggcctcagccgtgaggatgtaggccgtggataacctccataaaata

SEQ ID NO: 172 - DNA, 60 bp, synthetic construct
  misc_feature 1..60: note = Synthetic
  source 1..60: mol_type = other DNA
ggaacatcgaccactgagtgcctatgaggagctgttgtgacgaaagtatcttagcgggca

SEQ ID NO: 173 - DNA, 60 bp, synthetic construct
  misc_feature 1..60: note = Synthetic
  source 1..60: mol_type = other DNA
accgttatttgccattttcatgcccgctaagatactttcgtcacaacagctcctcatagg

SEQ ID NO: 174 - DNA, 60 bp, synthetic construct
  misc_feature 1..60: note = Synthetic
  source 1..60: mol_type = other DNA
gaacgatattgatcgagaagagccctgcgcagccgctgccggcgatgccccctcgacctc

SEQ ID NO: 175 - DNA, 60 bp, synthetic construct
  misc_feature 1..60: note = Synthetic
  source 1..60: mol_type = other DNA
tcctgaacgcctccctgatcgaggtcgagggggcatcgccggcagcggctgcgcagggct

SEQ ID NO: 176 - DNA, 60 bp, synthetic construct
  misc_feature 1..60: note = Synthetic
  source 1..60: mol_type = other DNA
ctcacaaagaaagccgggcaatgcccggctttttctgctgacgcctcctagatcgagcgc

SEQ ID NO: 177 - DNA, 60 bp, synthetic construct
  misc_feature 1..60: note = Synthetic
  source 1..60: mol_type = other DNA
aaggccgagcagaaacgctcgcgctcgatctaggaggcgtcagcagaaaaagccgggcat

SEQ ID NO: 178 - DNA, 60 bp, synthetic construct
  misc_feature 1..60: note = Synthetic
  source 1..60: mol_type = other DNA
gaaaatcgtgcgggtacgcctcgatgttcatacgcctcctacgctctagttctccttagt

SEQ ID NO: 179 - DNA, 60 bp, synthetic construct
  misc_feature 1..60: note = Synthetic
  source 1..60: mol_type = other DNA
gcaacgccgcgcgagaacctactaaggagaactagagcgtaggaggcgtatgaacatcga

SEQ ID NO: 180 - DNA, 60 bp, synthetic construct
  misc_feature 1..60: note = Synthetic
  source 1..60: mol_type = other DNA
gtgccccgatctgtactttgttcatacgctctagttctccagccaattacctcccgtcat

SEQ ID NO: 181 - DNA, 60 bp, synthetic construct
  misc_feature 1..60: note = Synthetic
  source 1..60: mol_type = other DNA
cacgacgaccgcggccgccaatgacgggaggtaattggctggagaactagagcgtatgaa

SEQ ID NO: 182 - DNA, 60 bp, synthetic construct
  misc_feature 1..60: note = Synthetic
  source 1..60: mol_type = other DNA
gtgccccgatctgtactttgttcatacgctctagttctccagttgccccctgcgcaggct

SEQ ID NO: 183 - DNA, 60 bp, synthetic construct
  misc_feature 1..60: note = Synthetic
  source 1..60: mol_type = other DNA
aacgacccagcaggccatcgagcctgcgcagggggcaactggagaactagagcgtatgaa

SEQ ID NO: 184 - DNA, 60 bp, synthetic construct
  misc_feature 1..60: note = Synthetic
  source 1..60: mol_type = other DNA
cctgctcgtggaacggctttttgacctctgccatagccaacgctttcactcctggttggt

SEQ ID NO: 185 - DNA, 60 bp, synthetic construct
  misc_feature 1..60: note = Synthetic
  source 1..60: mol_type = other DNA
acagcaaaggccgtaacggcaccaaccaggagtgaaagcgttggctatggcagaggtcaa

SEQ ID NO: 186 - DNA, 60 bp, synthetic construct
  misc_feature 1..60: note = Synthetic
  source 1..60: mol_type = other DNA
aagtcgtcctgctgcacggtcttgggatcattcatcgcttatatccccctaccctcacca

SEQ ID NO: 187 - DNA, 60 bp, synthetic construct
  misc_feature 1..60: note = Synthetic
  source 1..60: mol_type = other DNA
tcatcaggccggttctgacctggtgagggtagggggatataagcgatgaatgatcccaag

SEQ ID NO: 188 - DNA, 60 bp, synthetic construct
  misc_feature 1..60: note = Synthetic
  source 1..60: mol_type = other DNA
gcgattacaaggcgttcaaattgcatatatccccctaccctcatatcgtgatcccctccc

SEQ ID NO: 189 - DNA, 60 bp, synthetic construct
  misc_feature 1..60: note = Synthetic
  source 1..60: mol_type = other DNA
gacggccaccgtcgaggaaggggaggggatcacgatatgagggtagggggatatatgcaa

SEQ ID NO: 190 - DNA, 60 bp, synthetic construct
  misc_feature 1..60: note = Synthetic
  source 1..60: mol_type = other DNA
cgatggcgacgtacttggtgaggcgctggaagcggctcattcatctactcctacctcggg

SEQ ID NO: 191 - DNA, 60 bp, synthetic construct
  misc_feature 1..60: note = Synthetic
  source 1..60: mol_type = other DNA
gcgaggctcccttaaaactacccgaggtaggagtagatgaatgagccgcttccagcgcct

SEQ ID NO: 192 - DNA, 60 bp, synthetic construct
  misc_feature 1..60: note = Synthetic
  source 1..60: mol_type = other DNA
cccgtatctgtggccccacggcgttgtttcggttcttcatggggacgtgcttggcaatca

SEQ ID NO: 193 - DNA, 59 bp, synthetic construct
  misc_feature 1..59: note = Synthetic
  source 1..59: mol_type = other DNA
aacggccggggggtgcgcgtgattgccaagcacgtccccatgaagaaccgaaacaacgc

SEQ ID NO: 194 - DNA, 60 bp, synthetic construct
  misc_feature 1..60: note = Synthetic
  source 1..60: mol_type = other DNA
tcggcaacatatttctcggccgccgcgatctgttcgggctgtgcttgctccttcgtcagt

SEQ ID NO: 195 - DNA, 60 bp, synthetic construct
  misc_feature 1..60: note = Synthetic
  source 1..60: mol_type = other DNA
gagcgccgccgtcaagaactactgacgaaggagcaagcacagcccgaacagatcgcggcg

SEQ ID NO: 196 - DNA, 60 bp, synthetic construct
  misc_feature 1..60: note = Synthetic
  source 1..60: mol_type = other DNA
tcgctcttcttgatggagcgcatggggacgtgcttggcaaggctctgccctcgggcggac

SEQ ID NO: 197 - DNA, 60 bp, synthetic construct
  misc_feature 1..60: note = Synthetic
  source 1..60: mol_type = other DNA
gcaaggtcatgatgggcgtggtccgcccgagggcagagccttgccaagcacgtccccatg

SEQ ID NO: 198 - DNA, 60 bp, synthetic construct
  misc_feature 1..60: note = Synthetic
  source 1..60: mol_type = other DNA
aatcacgcgcaccccccggccgttttagcggctaaaaaagccgcttgccgaattctgacg

SEQ ID NO: 199 - DNA, 60 bp, synthetic construct
  misc_feature 1..60: note = Synthetic
  source 1..60: mol_type = other DNA
ttccttggtgtatccaacggcgtcagaattcggcaagcggcttttttagccgctaaaacg

SEQ ID NO: 200 - DNA, 60 bp, synthetic construct
  misc_feature 1..60: note = Synthetic
  source 1..60: mol_type = other DNA
atgcaggaaattactgaactgaggggacaggcgagagacgatggcgaaaattcacatggt

SEQ ID NO: 201 - DNA, 60 bp, synthetic construct
  misc_feature 1..60: note = Synthetic
  source 1..60: mol_type = other DNA
ccccgcccttgccctgcaaaaccatgtgaattttcgccatcgtctctcgcctgtcccctc

SEQ ID NO: 202 - DNA, 60 bp, synthetic construct
  misc_feature 1..60: note = Synthetic
  source 1..60: mol_type = other DNA
caccttcaacccaacaccggacaaaaaggatctactgtaaatgagcgaccagattgaaga

SEQ ID NO: 203 - DNA, 60 bp, synthetic construct
  misc_feature 1..60: note = Synthetic
  source 1..60: mol_type = other DNA
ccgcaatctcccggatcagctcttcaatctggtcgctcatttacagtagatcctttttgt

SEQ ID NO: 204 - DNA, 60 bp, synthetic construct
  misc_feature 1..60: note = Synthetic
  source 1..60: mol_type = other DNA
cggcctgtttgaacagctcgacgcggcggccgtgctatgaatcgcagaggcgcagatgaa

SEQ ID NO: 205 - DNA, 60 bp, synthetic construct
  misc_feature 1..60: note = Synthetic
  source 1..60: mol_type = other DNA
gcccggcaacgccgggctttttcatctgcgcctctgcgattcatagcacggccgccgcgt

SEQ ID NO: 206 - DNA, 60 bp, synthetic construct
  misc_feature 1..60: note = Synthetic
  source 1..60: mol_type = other DNA
gcagatgaaaaagcccggcgttgccgggcttgtttttgcggacgcctccctttttagccg

SEQ ID NO: 207 - DNA, 60 bp, synthetic construct
  misc_feature 1..60: note = Synthetic
  source 1..60: mol_type = other DNA
cgcactcgttagagttttagcggctaaaaagggaggcgtccgcaaaaacaagcccggcaa

SEQ ID NO: 208 - DNA, 60 bp, synthetic construct
  misc_feature 1..60: note = Synthetic
  source 1..60: mol_type = other DNA
ttgttccatctattttagtgaactgcgttcgatttatcaggttggctccggtaattggta

SEQ ID NO: 209 - DNA, 60 bp, synthetic construct
  misc_feature 1..60: note = Synthetic
  source 1..60: mol_type = other DNA
ggtaagagtattattattcttaccaattaccggagccaacctgataaatcgaacgcagtt

SEQ ID NO: 210 - DNA, 60 bp, synthetic construct
  misc_feature 1..60: note = Synthetic
  source 1..60: mol_type = other DNA
cttgcaacatcaggagccgtttcttttgttcgtcagtcattcttcggtcctccttgtagc

SEQ ID NO: 211 - DNA, 60 bp, synthetic construct
  misc_feature 1..60: note = Synthetic
  source 1..60: mol_type = other DNA
gccgcacagacaacggttccgctacaaggaggaccgaagaatgactgacgaacaaaagaa

SEQ ID NO: 212 - DNA, 60 bp, synthetic construct
  misc_feature 1..60: note = Synthetic
  source 1..60: mol_type = other DNA
aaaacaaaagcccggaaaccgggctttcgtctcttgccgctcatacggactcctgttggg

SEQ ID NO: 213 - DNA, 60 bp, synthetic construct
  misc_feature 1..60: note = Synthetic
  source 1..60: mol_type = other DNA
ccagcccgtgcgcgagctggcccaacaggagtccgtatgagcggcaagagacgaaagccc

SEQ ID NO: 214 - DNA, 60 bp, synthetic construct
  misc_feature 1..60: note = Synthetic
  source 1..60: mol_type = other DNA
aggccgcctacctgggcgaaaacatcggtgtttgtggcatgtcgaaggcgacgatagggg

SEQ ID NO: 215 - DNA, 60 bp, synthetic construct
  misc_feature 1..60: note = Synthetic
  source 1..60: mol_type = other DNA
aacgacgcacgacgccaaggcccctatcgtcgccttcgacatgccacaaacaccgatgtt

SEQ ID NO: 216 - DNA, 60 bp, synthetic construct
  misc_feature 1..60: note = Synthetic
  source 1..60: mol_type = other DNA
tgcgtcgttttcagtgcgttcatagggttctcccgccgtgtcgatacaccctcgcggtgg

SEQ ID NO: 217 - DNA, 60 bp, synthetic construct
  misc_feature 1..60: note = Synthetic
  source 1..60: mol_type = other DNA
tccatcgaaaagcaattaacccaccgcgagggtgtatcgacacggcgggagaaccctatg

SEQ ID NO: 218 - DNA, 60 bp, synthetic construct
  misc_feature 1..60: note = Synthetic
  source 1..60: mol_type = other DNA
ccgctaaaatttggggacaggtcatttacagaaagccagctgctcaaagctccttgaagg

SEQ ID NO: 219 - DNA, 60 bp, synthetic construct
  misc_feature 1..60: note = Synthetic
  source 1..60: mol_type = other DNA
ggagggtcaagcaagcggccccttcaaggagctttgagcagctggctttctgtaaatgac

SEQ ID NO: 220 - DNA, 60 bp, synthetic construct
  misc_feature 1..60: note = Synthetic
  source 1..60: mol_type = other DNA
ttagccgctaaaatttggggacaggtcatttacagaaagcgccccagccctcggcctccg

SEQ ID NO: 221 - DNA, 60 bp, synthetic construct
  misc_feature 1..60: note = Synthetic
  source 1..60: mol_type = other DNA
atcggcgtactggttcaatccggaggccgagggctggggcgctttctgtaaatgacctgt

SEQ ID NO: 222 - DNA, 60 bp, synthetic construct
  misc_feature 1..60: note = Synthetic
  source 1..60: mol_type = other DNA
cctgtcttggcattgctcaaagctccttgaaggggccgcttgcttgaccctccacggcga

SEQ ID NO: 223 - DNA, 60 bp, synthetic construct
  misc_feature 1..60: note = Synthetic
  source 1..60: mol_type = other DNA
ctggacgaattgaacacgcatcgccgtggagggtcaagcaagcggccccttcaaggagct

SEQ ID NO: 224 - DNA, 60 bp, synthetic construct
  misc_feature 1..60: note = Synthetic
  source 1..60: mol_type = other DNA
atcgaggtcaagcgccccggagaaatccggggcgtcatcccatcatcccctggcgtcagt

SEQ ID NO: 225 - DNA, 60 bp, synthetic construct
  misc_feature 1..60: note = Synthetic
  source 1..60: mol_type = other DNA
ggcgaaattccgggccggtcactgacgccaggggatgatgggatgacgccccggatttct

SEQ ID NO: 226 - DNA, 60 bp, synthetic construct
  misc_feature 1..60: note = Synthetic
  source 1..60: mol_type = other DNA
agttcaccctatctcctacttgcatcatcatcccctggcgcggattggcctccggtaatt

SEQ ID NO: 227 - DNA, 60 bp, synthetic construct
  misc_feature 1..60: note = Synthetic
  source 1..60: mol_type = other DNA
gccgggtagattcccaggtcaattaccggaggccaatccgcgccaggggatgatgatgca

SEQ ID NO: 228 - DNA, 60 bp, synthetic construct
  misc_feature 1..60: note = Synthetic
  source 1..60: mol_type = other DNA
gaggcgtcatgcttgaaaacacctttcccctggcgtgcaaggccctatctccttgagaga

SEQ ID NO: 229 - DNA, 60 bp, synthetic construct
  misc_feature 1..60: note = Synthetic
  source 1..60: mol_type = other DNA
ggcccggctacggtcgggcctctctcaaggagatagggccttgcacgccaggggaaaggt

SEQ ID NO: 230 - DNA, 60 bp, synthetic construct
  misc_feature 1..60: note = Synthetic
  source 1..60: mol_type = other DNA
ctccttgagagaggcccgaccgtagccgggcctcgttccgcggggttgatcctccggttg

SEQ ID NO: 231 - DNA, 60 bp, synthetic construct
  misc_feature 1..60: note = Synthetic
  source 1..60: mol_type = other DNA
gggcacttcgcccaggtcagcaaccggaggatcaaccccgcggaacgaggcccggctacg

SEQ ID NO: 232 - DNA, 40 bp, synthetic construct
  misc_feature 1..40: note = Synthetic
  source 1..40: mol_type = other DNA
tgccgccgcgcgcatggtcgtaatgggaccgatagcccgt

SEQ ID NO: 233 - DNA, 40 bp, synthetic construct
  misc_feature 1..40: note = Synthetic
  source 1..40: mol_type = other DNA
tttaacctacttcctttggttccgggggatctcgcgactc

SEQ ID NO: 234 - DNA, 20 bp, synthetic construct
  misc_feature 1..20: note = Synthetic
  source 1..20: mol_type = other DNA
atcgaagagaagcaggacga

SEQ ID NO: 235 - DNA, 20 bp, synthetic construct
  misc_feature 1..20: note = Synthetic
  source 1..20: mol_type = other DNA
tgctggtccatgaagatgaa

SEQ ID NO: 236 - DNA, 20 bp, synthetic construct
  misc_feature 1..20: note = Synthetic
  source 1..20: mol_type = other DNA
tcgagctgatgtttgacgac

SEQ ID NO: 237 - DNA, 20 bp, synthetic construct
  misc_feature 1..20: note = Synthetic
  source 1..20: mol_type = other DNA
ggacttgaggttgctctgct

SEQ ID NO: 238 - DNA, 20 bp, synthetic construct
  misc_feature 1..20: note = Synthetic
  source 1..20: mol_type = other DNA
gtggacattggtttcagcaa

SEQ ID NO: 239 - DNA, 60 bp, synthetic construct
  misc_feature 1..60: note = Synthetic
  source 1..60: mol_type = other DNA
gcgagaacctactaaggagaactagagcgtacgtgcttcgaaccactcggagggacggtt

SEQ ID NO: 240 - DNA, 60 bp, synthetic construct
  misc_feature 1..60: note = Synthetic
  source 1..60: mol_type = other DNA
aaccgtccctccgagtggttcgaagcacgtacgctctagttctccttagtaggttctcgc

SEQ ID NO: 241 - DNA, 20 bp, synthetic construct
  misc_feature 1..20: note = Synthetic
  source 1..20: mol_type = other DNA
aagcgatgaatgatcccaag

SEQ ID NO: 242 - DNA, 20 bp, synthetic construct
  misc_feature 1..20: note = Synthetic
  source 1..20: mol_type = other DNA
gagcaatggatagccgatgt

SEQ ID NO: 243 - DNA, 20 bp, synthetic construct
  misc_feature 1..20: note = Synthetic
  source 1..20: mol_type = other DNA
aagcgatgaatgatcccaag

SEQ ID NO: 244 - DNA, 20 bp, synthetic construct
  misc_feature 1..20: note = Synthetic
  source 1..20: mol_type = other DNA
tgtaacgcttcccggtagtc

SEQ ID NO: 245 - DNA, 20 bp, synthetic construct
  misc_feature 1..20: note = Synthetic
  source 1..20: mol_type = other DNA
cattgcaaagcgactgatgt

SEQ ID NO: 246 - DNA, 20 bp, synthetic construct
  misc_feature 1..20: note = Synthetic
  source 1..20: mol_type = other DNA
gatccgctccttgaactctg

SEQ ID NO: 247 - DNA, 18 bp, synthetic construct
  misc_feature 1..18: note = Synthetic
  source 1..18: mol_type = other DNA
aggcccttgccaatgaat

SEQ ID NO: 248 - DNA, 40 bp, synthetic construct
  misc_feature 1..40: note = Synthetic
  source 1..40: mol_type = other DNA
ttctttgaatgcgcgggcgtcctggtgagcgtagtccagc

SEQ ID NO: 249 - DNA, 40 bp, synthetic construct
  misc_feature 1..40: note = Synthetic
  source 1..40: mol_type = other DNA
cgttcccgcctgcccctgattggcccgctgatcgaccgct

SEQ ID NO: 250 - DNA, 19 bp, synthetic construct
  misc_feature 1..19: note = Synthetic
  source 1..19: mol_type = other DNA
aatgttgcaaggcgatcag

SEQ ID NO: 251 - DNA, 20 bp, synthetic construct
  misc_feature 1..20: note = Synthetic
  source 1..20: mol_type = other DNA
agccctcccgtatcgtagtt

SEQ ID NO: 252 - DNA, 40 bp, synthetic construct; identical to SEQ ID NO: 232
  misc_feature 1..40: note = Synthetic
  source 1..40: mol_type = other DNA

SEQ ID NO: 253 - DNA, 40 bp, synthetic construct; identical to SEQ ID NO: 233
  misc_feature 1..40: note = Synthetic
  source 1..40: mol_type = other DNA

SEQ ID NO: 254 - DNA, 20 bp, synthetic construct
  misc_feature 1..20: note = Synthetic
  source 1..20: mol_type = other DNA
atcgaagagaagcaggacga

SEQ ID NO: 255 - DNA, 20 bp, synthetic construct
  misc_feature 1..20: note = Synthetic
  source 1..20: mol_type = other DNA
tgctggtccatgaagatgaa

SEQ ID NO: 256 - DNA, 20 bp, synthetic construct
  misc_feature 1..20: note = Synthetic
  source 1..20: mol_type = other DNA
tcgagctgatgtttgacgac

SEQ ID NO: 257 - DNA, 20 bp, synthetic construct
  misc_feature 1..20: note = Synthetic
  source 1..20: mol_type = other DNA
ggacttgaggttgctctgct

SEQ ID NO: 258 - DNA, 40 bp, synthetic construct
  misc_feature 1..40: note = Synthetic
  source 1..40: mol_type = other DNA
ggaccaggcgcagtccaccatcaacggcctgatgagcgcc

SEQ ID NO: 259 - DNA, 25 bp, synthetic construct
  misc_feature 1..25: note = Synthetic
  source 1..25: mol_type = other DNA
atcggcgtgaagcccaacagggcca

SEQ ID NO: 260 - DNA, 20 bp, synthetic construct
  misc_feature 1..20: note = Synthetic
  source 1..20: mol_type = other DNA
gtggacattggtttcagcaa

SEQ ID NO: 261 - DNA, 20 bp, synthetic construct
  misc_feature 1..20: note = Synthetic
  source 1..20: mol_type = other DNA
agctcatgcatcacaacagc

SEQ ID NO: 262 - DNA, 20 bp, synthetic construct
  misc_feature 1..20: note = Synthetic
  source 1..20: mol_type = other DNA
gagcaatggatagccgatgt

SEQ ID NO: 263 - DNA, 20 bp, synthetic construct
  misc_feature 1..20: note = Synthetic
  source 1..20: mol_type = other DNA
aagcgatgaatgatcccaag

SEQ ID NO: 264 - DNA, 20 bp, synthetic construct
  misc_feature 1..20: note = Synthetic
  source 1..20: mol_type = other DNA
tgtaacgcttcccggtagtc

SEQ ID NO: 265 - DNA, 20 bp, synthetic construct
  misc_feature 1..20: note = Synthetic
  source 1..20: mol_type = other DNA
cattgcaaagcgactgatgt

SEQ ID NO: 266 - DNA, 20 bp, synthetic construct
  misc_feature 1..20: note = Synthetic
  source 1..20: mol_type = other DNA
gatccgctccttgaactctg

SEQ ID NO: 267 - DNA, 18 bp, synthetic construct
  misc_feature 1..18: note = Synthetic
  source 1..18: mol_type = other DNA
aggcccttgccaatgaat

SEQ ID NO: 268 - DNA, 25 bp, synthetic construct
  misc_feature 1..25: note = Synthetic
  source 1..25: mol_type = other DNA
ttctttgaatgcgcgggcgtcctgg

SEQ ID NO: 269 - DNA, 60 bp, synthetic construct
  misc_feature 1..60: note = Synthetic
  source 1..60: mol_type = other DNA
tgaacacgcatcgccgtggagggtcaagcagcggcaagagacgaaagcccggtttccggg

SEQ ID NO: 270 - DNA, 60 bp, synthetic construct
  misc_feature 1..60: note = Synthetic
  source 1..60: mol_type = other DNA
cccggaaaccgggctttcgtctcttgccgctgcttgaccctccacggcgatgcgtgttca

SEQ ID NO: 271 - DNA, 20 bp, synthetic construct
  misc_feature 1..20: note = Synthetic
  source 1..20: mol_type = other DNA
agccctcccgtatcgtagtt

SEQ ID NO: 272 - DNA, 40 bp, synthetic construct; identical to SEQ ID NO: 232
  misc_feature 1..40: note = Synthetic
  source 1..40: mol_type = other DNA

SEQ ID NO: 273 - DNA, 40 bp, synthetic construct; identical to SEQ ID NO: 233
  misc_feature 1..40: note = Synthetic
  source 1..40: mol_type = other DNA

SEQ ID NO: 274 - DNA, 20 bp, synthetic construct
  misc_feature 1..20: note = Synthetic
  source 1..20: mol_type = other DNA
atcgaagagaagcaggacga

SEQ ID NO: 275 - DNA, 20 bp, synthetic construct
  misc_feature 1..20: note = Synthetic
  source 1..20: mol_type = other DNA
tgctggtccatgaagatgaa

SEQ ID NO: 276 - DNA, 20 bp, synthetic construct
  misc_feature 1..20: note = Synthetic
  source 1..20: mol_type = other DNA
tcgagctgatgtttgacgac

SEQ ID NO: 277 - DNA, 20 bp, synthetic construct
  misc_feature 1..20: note = Synthetic
  source 1..20: mol_type = other DNA
ggacttgaggttgctctgct

SEQ ID NO: 278 - DNA, 40 bp, synthetic construct; identical to SEQ ID NO: 258
  misc_feature 1..40: note = Synthetic
  source 1..40: mol_type = other DNA

SEQ ID NO: 279 - DNA, 25 bp, synthetic construct
  misc_feature 1..25: note = Synthetic
  source 1..25: mol_type = other DNA
atcggcgtgaagcccaacagggcca

SEQ ID NO: 280 - DNA, 20 bp, synthetic construct
  misc_feature 1..20: note = Synthetic
  source 1..20: mol_type = other DNA
gtggacattggtttcagcaa

SEQ ID NO: 281 - DNA, 60 bp, synthetic construct; identical to SEQ ID NO: 239
  misc_feature 1..60: note = Synthetic
  source 1..60: mol_type = other DNA

SEQ ID NO: 282 - DNA, 60 bp, synthetic construct; identical to SEQ ID NO: 240
  misc_feature 1..60: note = Synthetic
  source 1..60: mol_type = other DNA

SEQ ID NO: 283 - DNA, 20 bp, synthetic construct
  misc_feature 1..20: note = Synthetic
  source 1..20: mol_type = other DNA
aagcgatgaatgatcccaag

SEQ ID NO: 284 - DNA, 20 bp, synthetic construct
  misc_feature 1..20: note = Synthetic
  source 1..20: mol_type = other DNA
tgtaacgcttcccggtagtc

SEQ ID NO: 285 - DNA, 20 bp, synthetic construct
  misc_feature 1..20: note = Synthetic
  source 1..20: mol_type = other DNA
cattgcaaagcgactgatgt

SEQ ID NO: 286 - DNA, 20 bp, synthetic construct
  misc_feature 1..20: note = Synthetic
  source 1..20: mol_type = other DNA
gatccgctccttgaactctg

SEQ ID NO: 287 - DNA, 18 bp, synthetic construct
  misc_feature 1..18: note = Synthetic
  source 1..18: mol_type = other DNA
aggcccttgccaatgaat

SEQ ID NO: 288 - DNA, 40 bp, synthetic construct; identical to SEQ ID NO: 248
  misc_feature 1..40: note = Synthetic
  source 1..40: mol_type = other DNA

SEQ ID NO: 289 - DNA, 40 bp, synthetic construct; identical to SEQ ID NO: 249
  misc_feature 1..40: note = Synthetic
  source 1..40: mol_type = other DNA

SEQ ID NO: 290 - DNA, 19 bp, synthetic construct
  misc_feature 1..19: note = Synthetic
  source 1..19: mol_type = other DNA
aatgttgcaaggcgatcag

SEQ ID NO: 291 - DNA, 20 bp, synthetic construct
  misc_feature 1..20: note = Synthetic
  source 1..20: mol_type = other DNA
agccctcccgtatcgtagtt

SEQ ID NO: 292 - DNA, 40 bp, synthetic construct; identical to SEQ ID NO: 232
  misc_feature 1..40: note = Synthetic
  source 1..40: mol_type = other DNA

SEQ ID NO: 293 - DNA, 40 bp, synthetic construct; identical to SEQ ID NO: 233
  misc_feature 1..40: note = Synthetic
  source 1..40: mol_type = other DNA

SEQ ID NO: 294 - DNA, 20 bp, synthetic construct
  misc_feature 1..20: note = Synthetic
  source 1..20: mol_type = other DNA
atcgaagagaagcaggacga

SEQ ID NO: 295 - DNA, 20 bp, synthetic construct
  misc_feature 1..20: note = Synthetic
  source 1..20: mol_type = other DNA
tgctggtccatgaagatgaa

SEQ ID NO: 296 - DNA, 20 bp, synthetic construct
  misc_feature 1..20: note = Synthetic
  source 1..20: mol_type = other DNA
tcgagctgatgtttgacgac

SEQ ID NO: 297 - DNA, 20 bp, synthetic construct
  misc_feature 1..20: note = Synthetic
  source 1..20: mol_type = other DNA
ggacttgaggttgctctgct

SEQ ID NO: 298 - DNA, 40 bp, synthetic construct; identical to SEQ ID NO: 258
  misc_feature 1..40: note = Synthetic
  source 1..40: mol_type = other DNA

SEQ ID NO: 299 - DNA, 25 bp, synthetic construct
  misc_feature 1..25: note = Synthetic
  source 1..25: mol_type = other DNA
atcggcgtgaagcccaacagggcca

SEQ ID NO: 300 - DNA, 20 bp, synthetic construct
  misc_feature 1..20: note = Synthetic
  source 1..20: mol_type = other DNA
gtggacattggtttcagcaa

SEQ ID NO: 301 - DNA, 60 bp, synthetic construct
  misc_feature 1..60: note = Synthetic
  source 1..60: mol_type = other DNA
ggttctgacctggtgagggtagggggatatacgtgcttcgaaccactcggagggacggtt

SEQ ID NO: 302 - DNA, 60 bp, synthetic construct
  misc_feature 1..60: note = Synthetic
  source 1..60: mol_type = other DNA
aaccgtccctccgagtggttcgaagcacgtatatccccctaccctcaccaggtcagaacc

SEQ ID NO: 303 - DNA, 20 bp, synthetic construct
  misc_feature 1..20: note = Synthetic
  source 1..20: mol_type = other DNA
tgtaacgcttcccggtagtc

SEQ ID NO: 304 - DNA, 20 bp, synthetic construct
  misc_feature 1..20: note = Synthetic
  source 1..20: mol_type = other DNA
gatccgctccttgaactctg

SEQ ID NO: 305 - DNA, 18 bp, synthetic construct
  misc_feature 1..18: note = Synthetic
  source 1..18: mol_type = other DNA
aggcccttgccaatgaat

SEQ ID NO: 306 - DNA, 40 bp, synthetic construct; identical to SEQ ID NO: 248
  misc_feature 1..40: note = Synthetic
  source 1..40: mol_type = other DNA

SEQ ID NO: 307 - DNA, 40 bp, synthetic construct; identical to SEQ ID NO: 249
  misc_feature 1..40: note = Synthetic
  source 1..40: mol_type = other DNA

SEQ ID NO: 308 - DNA, 19 bp, synthetic construct
  misc_feature 1..19: note = Synthetic
  source 1..19: mol_type = other DNA
aatgttgcaaggcgatcag

SEQ ID NO: 309 - DNA, 20 bp, synthetic construct
  misc_feature 1..20: note = Synthetic
  source 1..20: mol_type = other DNA
agccctcccgtatcgtagtt

SEQ ID NO: 310 - DNA, 40 bp, synthetic construct; identical to SEQ ID NO: 232
  misc_feature 1..40: note = Synthetic
  source 1..40: mol_type = other DNA

SEQ ID NO: 311 - DNA, 20 bp, synthetic construct
  misc_feature 1..20: note = Synthetic
  source 1..20: mol_type = other DNA
tgctggtccatgaagatgaa

SEQ ID NO: 312 - DNA, 20 bp, synthetic construct
  misc_feature 1..20: note = Synthetic
  source 1..20: mol_type = other DNA
tcgagctgatgtttgacgac

SEQ ID NO: 313 - DNA, 60 bp, synthetic construct
  misc_feature 1..60: note = Synthetic
  source 1..60: mol_type = other DNA
tcatcaggccggttctgacctggtgagggtagggggatatcgctatcccctccccttacc

SEQ ID NO: 314 - DNA, 60 bp, synthetic construct
  misc_feature 1..60: note = Synthetic
  source 1..60: mol_type = other DNA
ggtaaggggaggggatagcgatatccccctaccctcaccaggtcagaaccggcctgatga

SEQ ID NO: 315 - DNA, 20 bp, synthetic construct
  misc_feature 1..20: note = Synthetic
  source 1..20: mol_type = other DNA
cattgcaaagcgactgatgt

SEQ ID NO: 316 - DNA, 20 bp, synthetic construct
  misc_feature 1..20: note = Synthetic
  source 1..20: mol_type = other DNA
gatccgctccttgaactctg

SEQ ID NO: 317 - DNA, 18 bp, synthetic construct
  misc_feature 1..18: note = Synthetic
  source 1..18: mol_type = other DNA
aggcccttgccaatgaat

SEQ ID NO: 318 - DNA, 40 bp, synthetic construct; identical to SEQ ID NO: 248
  misc_feature 1..40: note = Synthetic
  source 1..40: mol_type = other DNA

SEQ ID NO: 319 - DNA, 20 bp, synthetic construct
  misc_feature 1..20: note = Synthetic
  source 1..20: mol_type = other DNA
agccctcccgtatcgtagtt

SEQ ID NO: 320 - DNA, 60 bp, synthetic construct
  misc_feature 1..60: note = Synthetic
  source 1..60: mol_type = other DNA
aaaacaaaagcccggaaaccgggctttcgtctcttgccgccggggttgatcctccggttg

SEQ ID NO: 321 - DNA, 20 bp, synthetic construct
  misc_feature 1..20: note = Synthetic
  source 1..20: mol_type = other DNA
tgctggtccatgaagatgaa

SEQ ID NO: 322 - DNA, 20 bp, synthetic construct
  misc_feature 1..20: note = Synthetic
  source 1..20: mol_type = other DNA
tcgagctgatgtttgacgac

SEQ ID NO: 323 - DNA, 60 bp, synthetic construct; identical to SEQ ID NO: 313
  misc_feature 1..60: note = Synthetic
  source 1..60: mol_type = other DNA

SEQ ID NO: 324 - DNA, 60 bp, synthetic construct; identical to SEQ ID NO: 314
  misc_feature 1..60: note = Synthetic
  source 1..60: mol_type = other DNA

SEQ ID NO: 325 - DNA, 20 bp, synthetic construct
  misc_feature 1..20: note = Synthetic
  source 1..20: mol_type = other DNA
cattgcaaagcgactgatgt

SEQ ID NO: 326 - DNA, 20 bp, synthetic construct
  misc_feature 1..20: note = Synthetic
  source 1..20: mol_type = other DNA
gatccgctccttgaactctg

SEQ ID NO: 327 - DNA, 18 bp, synthetic construct
  misc_feature 1..18: note = Synthetic
  source 1..18: mol_type = other DNA
aggcccttgccaatgaat

SEQ ID NO: 328 - DNA, 40 bp, synthetic construct; identical to SEQ ID NO: 248
  misc_feature 1..40: note = Synthetic
  source 1..40: mol_type = other DNA

SEQ ID NO: 329 - DNA, 60 bp, synthetic construct
  misc_feature 1..60: note = Synthetic
  source 1..60: mol_type = other DNA
gggcacttcgcccaggtcagcaaccggaggatcaaccccggcggcaagagacgaaagccc

SEQ ID NO: 330 - DNA, 40 bp, synthetic construct; identical to SEQ ID NO: 232
  misc_feature 1..40: note = Synthetic
  source 1..40: mol_type = other DNA

SEQ ID NO: 331 - DNA, 60 bp, synthetic construct
  misc_feature 1..60: note = Synthetic
  source 1..60: mol_type = other DNA
tgccccggcgtgagtcggggcaatcccgcaaggagggtgaccgcttgccctcatctgtta

SEQ ID NO: 332 - DNA, 60 bp, synthetic construct
  misc_feature 1..60: note = Synthetic
  source 1..60: mol_type = other DNA
ctggccggctaccgccggcgtaacagatgagggcaagcggtcaccctccttgcgggattg

SEQ ID NO: 333 - DNA, 20 bp, synthetic construct
  misc_feature 1..20: note = Synthetic
  source 1..20: mol_type = other DNA
tgctggtccatgaagatgaa

SEQ ID NO: 334 - DNA, 20 bp, synthetic construct
  misc_feature 1..20: note = Synthetic
  source 1..20: mol_type = other DNA
tcgagctgatgtttgacgac

SEQ ID NO: 335 - DNA, 60 bp, synthetic construct; identical to SEQ ID NO: 313
  misc_feature 1..60: note = Synthetic
  source 1..60: mol_type = other DNA

SEQ ID NO: 336 - DNA, 60 bp, synthetic construct; identical to SEQ ID NO: 314
  misc_feature 1..60: note = Synthetic
  source 1..60: mol_type = other DNA

SEQ ID NO: 337 - DNA, 20 bp, synthetic construct
  misc_feature 1..20: note = Synthetic
  source 1..20: mol_type = other DNA
cattgcaaagcgactgatgt

SEQ ID NO: 338 - DNA, 20 bp, synthetic construct
  misc_feature 1..20: note = Synthetic
  source 1..20: mol_type = other DNA
gatccgctccttgaactctg

SEQ ID NO: 339 - DNA, 18 bp, synthetic construct
  misc_feature 1..18: note = Synthetic
  source 1..18: mol_type = other DNA
aggcccttgccaatgaat

SEQ ID NO: 340 - DNA, 40 bp, synthetic construct; identical to SEQ ID NO: 248
  misc_feature 1..40: note = Synthetic
  source 1..40: mol_type = other DNA

SEQ ID NO: 341 - DNA, 20 bp, synthetic construct
  misc_feature 1..20: note = Synthetic
  source 1..20: mol_type = other DNA
agccctcccgtatcgtagtt

SEQ ID NO: 342 - DNA, 60 bp, synthetic construct; identical to SEQ ID NO: 320
  misc_feature 1..60: note = Synthetic
  source 1..60: mol_type = other DNA

SEQ ID NO: 343 - DNA, 60 bp, synthetic construct; identical to SEQ ID NO: 331
  misc_feature 1..60: note = Synthetic
  source 1..60: mol_type = other DNA

SEQ ID NO: 344 - DNA, 60 bp, synthetic construct; identical to SEQ ID NO: 332
  misc_feature 1..60: note = Synthetic
  source 1..60: mol_type = other DNA

SEQ ID NO: 345 - DNA, 60 bp, synthetic construct
  misc_feature 1..60: note = Synthetic
  source 1..60: mol_type = other DNA
tgacctggtgagggtagggggatatcgctatcccctccccggtcgtagctcctaagagtc

SEQ ID NO: 346 - DNA, 60 bp, synthetic construct
  misc_feature 1..60: note = Synthetic
  source 1..60: mol_type = other DNA
agcccgcccaatcctgaaacgactcttaggagctacgaccggggaggggatagcgatatc

SEQ ID NO: 347 - DNA, 20 bp, synthetic construct
  misc_feature 1..20: note = Synthetic
  source 1..20: mol_type = other DNA
cattgcaaagcgactgatgt

SEQ ID NO: 348 - DNA, 20 bp, synthetic construct
  misc_feature 1..20: note = Synthetic
  source 1..20: mol_type = other DNA
gatccgctccttgaactctg

SEQ ID NO: 349 - DNA, 18 bp, synthetic construct
  misc_feature 1..18: note = Synthetic
  source 1..18: mol_type = other DNA
aggcccttgccaatgaat

SEQ ID NO: 350 - DNA, 40 bp, synthetic construct; identical to SEQ ID NO: 248
  misc_feature 1..40: note = Synthetic
  source 1..40: mol_type = other DNA

SEQ ID NO: 351 - DNA, 60 bp, synthetic construct; identical to SEQ ID NO: 329
  misc_feature 1..60: note = Synthetic
  source 1..60: mol_type = other DNA

SEQ ID NO: 352 - DNA, 40 bp, synthetic construct; identical to SEQ ID NO: 232
  misc_feature 1..40: note = Synthetic
  source 1..40: mol_type = other DNA

SEQ ID NO: 353 - DNA, 20 bp, synthetic construct
  misc_feature 1..20: note = Synthetic
  source 1..20: mol_type = other DNA
tgctggtccatgaagatgaa

SEQ ID NO: 354 - DNA, 20 bp, synthetic construct
  misc_feature 1..20: note = Synthetic
  source 1..20: mol_type = other DNA
tcgagctgatgtttgacgac

SEQ ID NO: 355 - DNA, 25 bp, synthetic construct
  misc_feature 1..25: note = Synthetic
  source 1..25: mol_type = other DNA
atcggcgtgaagcccaacagggcca

SEQ ID NO: 356 - DNA, 20 bp, synthetic construct
  misc_feature 1..20: note = Synthetic
  source 1..20: mol_type = other DNA
gtggacattggtttcagcaa

SEQ ID NO: 357 - DNA, 20 bp, synthetic construct
  misc_feature 1..20: note = Synthetic
  source 1..20: mol_type = other DNA
aagcgatgaatgatcccaag

SEQ ID NO: 358 - DNA, 20 bp, synthetic construct
  misc_feature 1..20: note = Synthetic
  source 1..20: mol_type = other DNA
tgtaacgcttcccggtagtc

SEQ ID NO: 359 - DNA, 18 bp, synthetic construct
  misc_feature 1..18: note = Synthetic
  source 1..18: mol_type = other DNA
aggcccttgccaatgaat

SEQ ID NO: 360 - DNA, 40 bp, synthetic construct; identical to SEQ ID NO: 248
  misc_feature 1..40: note = Synthetic
  source 1..40: mol_type = other DNA

SEQ ID NO: 361 - DNA, 20 bp, synthetic construct
  misc_feature 1..20: note = Synthetic
  source 1..20: mol_type = other DNA
agccctcccgtatcgtagtt

SEQ ID NO: 362 - DNA, 40 bp, synthetic construct; identical to SEQ ID NO: 232
  misc_feature 1..40: note = Synthetic
  source 1..40: mol_type = other DNA

SEQ ID NO: 363 - DNA, 20 bp, synthetic construct
  misc_feature 1..20: note = Synthetic
  source 1..20: mol_type = other DNA
tgctggtccatgaagatgaa

SEQ ID NO: 364 - DNA, 20 bp, synthetic construct
  misc_feature 1..20: note = Synthetic
  source 1..20: mol_type = other DNA
tcgagctgatgtttgacgac

SEQ ID NO: 365 - DNA, 25 bp, synthetic construct
  misc_feature 1..25: note = Synthetic
  source 1..25: mol_type = other DNA
atcggcgtgaagcccaacagggcca

SEQ ID NO: 366 - DNA, 20 bp, synthetic construct
  misc_feature 1..20: note = Synthetic
  source 1..20: mol_type = other DNA
gtggacattggtttcagcaa

SEQ ID NO: 367 - DNA, 20 bp, synthetic construct
  misc_feature 1..20: note = Synthetic
  source 1..20: mol_type = other DNA
aagcgatgaatgatcccaag

SEQ ID NO: 368 - DNA, 20 bp, synthetic construct
  misc_feature 1..20: note = Synthetic
  source 1..20: mol_type = other DNA
tgtaacgcttcccggtagtc

SEQ ID NO: 369 - DNA, 18 bp, synthetic construct
  misc_feature 1..18: note = Synthetic
  source 1..18: mol_type = other DNA
aggcccttgccaatgaat

SEQ ID NO: 370 - DNA, 40 bp, synthetic construct; identical to SEQ ID NO: 248
  misc_feature 1..40: note = Synthetic
  source 1..40: mol_type = other DNA

SEQ ID NO: 371 - DNA, 20 bp, synthetic construct
  misc_feature 1..20: note = Synthetic
  source 1..20: mol_type = other DNA
agccctcccgtatcgtagtt

SEQ ID NO: 372 - DNA, 40 bp, synthetic construct; identical to SEQ ID NO: 232
  misc_feature 1..40: note = Synthetic
  source 1..40: mol_type = other DNA

SEQ ID NO: 373 - DNA, 20 bp, synthetic construct
  misc_feature 1..20: note = Synthetic
  source 1..20: mol_type = other DNA
tgctggtccatgaagatgaa

SEQ ID NO: 374 - DNA, 20 bp, synthetic construct
  misc_feature 1..20: note = Synthetic
  source 1..20: mol_type = other DNA
tcgagctgatgtttgacgac

SEQ ID NO: 375 - DNA, 25 bp, synthetic construct
  misc_feature 1..25: note = Synthetic
  source 1..25: mol_type = other DNA
atcggcgtgaagcccaacagggcca

SEQ ID NO: 376 - DNA, 20 bp, synthetic construct
  misc_feature 1..20: note = Synthetic
  source 1..20: mol_type = other DNA
gtggacattggtttcagcaa

SEQ ID NO: 377 - DNA, 20 bp, synthetic construct
  misc_feature 1..20: note = Synthetic
  source 1..20: mol_type = other DNA
aagcgatgaatgatcccaag

SEQ ID NO: 378 - DNA, 20 bp, synthetic construct
  misc_feature 1..20: note = Synthetic
  source 1..20: mol_type = other DNA
tgtaacgcttcccggtagtc

SEQ ID NO: 379 - DNA, 18 bp, synthetic construct
  misc_feature 1..18: note = Synthetic
  source 1..18: mol_type = other DNA
aggcccttgccaatgaat

SEQ ID NO: 380 - DNA, 40 bp, synthetic construct; identical to SEQ ID NO: 248
  misc_feature 1..40: note = Synthetic
  source 1..40: mol_type = other DNA

SEQ ID NO: 381 - DNA, 20 bp, synthetic construct
  misc_feature 1..20: note = Synthetic
  source 1..20: mol_type = other DNA
agccctcccgtatcgtagtt

SEQ ID NO: 382 - DNA, 40 bp, synthetic construct; identical to SEQ ID NO: 232
  misc_feature 1..40: note = Synthetic
  source 1..40: mol_type = other DNA

SEQ ID NO: 383 - DNA, 20 bp, synthetic construct
  misc_feature 1..20: note = Synthetic
  source 1..20: mol_type = other DNA
tgctggtccatgaagatgaa

SEQ ID NO: 384 - DNA, 20 bp, synthetic construct
  misc_feature 1..20: note = Synthetic
  source 1..20: mol_type = other DNA
tcgagctgatgtttgacgac

SEQ ID NO: 385 - DNA, 25 bp, synthetic construct
  misc_feature 1..25: note = Synthetic
  source 1..25: mol_type = other DNA
atcggcgtgaagcccaacagggcca

SEQ ID NO: 386 - DNA, 20 bp, synthetic construct
  misc_feature 1..20: note = Synthetic
  source 1..20: mol_type = other DNA
gtggacattggtttcagcaa

SEQ ID NO: 387 - DNA, 20 bp, synthetic construct
  misc_feature 1..20: note = Synthetic
  source 1..20: mol_type = other DNA
aagcgatgaatgatcccaag

SEQ ID NO: 388 - DNA, 20 bp, synthetic construct
  misc_feature 1..20: note = Synthetic
  source 1..20: mol_type = other DNA
tgtaacgcttcccggtagtc

SEQ ID NO: 389 - DNA, 18 bp, synthetic construct
  misc_feature 1..18: note = Synthetic
  source 1..18: mol_type = other DNA
aggcccttgccaatgaat

SEQ ID NO: 390 - DNA, 40 bp, synthetic construct; identical to SEQ ID NO: 248
  misc_feature 1..40: note = Synthetic
  source 1..40: mol_type = other DNA

SEQ ID NO: 391 - DNA, 20 bp, synthetic construct
  misc_feature 1..20: note = Synthetic
  source 1..20: mol_type = other DNA
agccctcccgtatcgtagtt

SEQ ID NO: 392 - DNA, 40 bp, synthetic construct; identical to SEQ ID NO: 232
  misc_feature 1..40: note = Synthetic
  source 1..40: mol_type = other DNA

SEQ ID NO: 393 - DNA, 20 bp, synthetic construct
  misc_feature 1..20: note = Synthetic
  source 1..20: mol_type = other DNA
tgctggtccatgaagatgaa

SEQ ID NO: 394 - DNA, 20 bp, synthetic construct
  misc_feature 1..20: note = Synthetic
  source 1..20: mol_type = other DNA
tcgagctgatgtttgacgac

SEQ ID NO: 395 - DNA, 25 bp, synthetic construct
  misc_feature 1..25: note = Synthetic
  source 1..25: mol_type = other DNA
atcggcgtgaagcccaacagggcca

SEQ ID NO: 396 - DNA, 20 bp, synthetic construct
  misc_feature 1..20: note = Synthetic
  source 1..20: mol_type = other DNA
gtggacattggtttcagcaa

SEQ ID NO: 397 - DNA, 20 bp, synthetic construct
  misc_feature 1..20: note = Synthetic
  source 1..20: mol_type = other DNA
aagcgatgaatgatcccaag

SEQ ID NO: 398 - DNA, 20 bp, synthetic construct
  misc_feature 1..20: note = Synthetic
  source 1..20: mol_type = other DNA
tgtaacgcttcccggtagtc

SEQ ID NO: 399 - DNA, 18 bp, synthetic construct
  misc_feature 1..18: note = Synthetic
  source 1..18: mol_type = other DNA
aggcccttgccaatgaat

SEQ ID NO: 400 - DNA, 40 bp, synthetic construct; identical to SEQ ID NO: 248
  misc_feature 1..40: note = Synthetic
  source 1..40: mol_type = other DNA

SEQ ID NO: 401 - DNA, 20 bp, synthetic construct
  misc_feature 1..20: note = Synthetic
  source 1..20: mol_type = other DNA
agccctcccgtatcgtagtt

SEQ ID NO: 402 - DNA, 40 bp, synthetic construct; identical to SEQ ID NO: 232
  misc_feature 1..40: note = Synthetic
  source 1..40: mol_type = other DNA

SEQ ID NO: 403 - DNA, 40 bp, synthetic construct; identical to SEQ ID NO: 233
  misc_feature 1..40: note = Synthetic
  source 1..40: mol_type = other DNA

SEQ ID NO: 404 - DNA, 20 bp, synthetic construct
  misc_feature 1..20: note = Synthetic
  source 1..20: mol_type = other DNA
atcgaagagaagcaggacga

SEQ ID NO: 405 - DNA, 20 bp, synthetic construct
  misc_feature 1..20: note = Synthetic
  source 1..20: mol_type = other DNA
tgctggtccatgaagatgaa

SEQ ID NO: 406 - DNA, 20 bp, synthetic construct
  misc_feature 1..20: note = Synthetic
  source 1..20: mol_type = other DNA
tcgagctgatgtttgacgac

SEQ ID NO: 407 - DNA, 20 bp, synthetic construct
  misc_feature 1..20: note = Synthetic
  source 1..20: mol_type = other DNA
ggacttgaggttgctctgct

SEQ ID NO: 408 - DNA, 40 bp, synthetic construct; identical to SEQ ID NO: 258
  misc_feature 1..40: note = Synthetic
  source 1..40: mol_type = other DNA

SEQ ID NO: 409 - DNA, 25 bp, synthetic construct
  misc_feature 1..25: note = Synthetic
  source 1..25: mol_type = other DNA
atcggcgtgaagcccaacagggcca

SEQ ID NO: 410 - DNA, 20 bp, synthetic construct
  misc_feature 1..20: note = Synthetic
  source 1..20: mol_type = other DNA
gtggacattggtttcagcaa

SEQ ID NO: 411 - DNA, 20 bp, synthetic construct
  misc_feature 1..20: note = Synthetic
  source 1..20: mol_type = other DNA
agctcatgcatcacaacagc

SEQ ID NO: 412 - DNA, 20 bp, synthetic construct
  misc_feature 1..20: note = Synthetic
  source 1..20: mol_type = other DNA
gagcaatggatagccgatgt

SEQ ID NO: 413 - DNA, 20 bp, synthetic construct
  misc_feature 1..20: note = Synthetic
  source 1..20: mol_type = other DNA
aagcgatgaatgatcccaag

SEQ ID NO: 414 - DNA, 20 bp, synthetic construct
  misc_feature 1..20: note = Synthetic
  source 1..20: mol_type = other DNA
tgtaacgcttcccggtagtc

SEQ ID NO: 415 - DNA, 20 bp, synthetic construct
  misc_feature 1..20: note = Synthetic
  source 1..20: mol_type = other DNA
cattgcaaagcgactgatgt

SEQ ID NO: 416 - DNA, 20 bp, synthetic construct
  misc_feature 1..20: note = Synthetic
  source 1..20: mol_type = other DNA
gatccgctccttgaactctg

SEQ ID NO: 417 - DNA, 60 bp, synthetic construct
  misc_feature 1..60: note = Synthetic
  source 1..60: mol_type = other DNA
atccaacggcgtcagccgagggcaagcggatggctgatgaaaccaagccaaccaggaagg

SEQ ID NO: 418 - DNA, 60 bp, synthetic construct
  misc_feature 1..60: note = Synthetic
  source 1..60: mol_type = other DNA
ccttcctggttggcttggtttcatcagccatccgcttgccctcggctgacgccgttggat

SEQ ID NO: 419 - DNA, 18 bp, synthetic construct
  misc_feature 1..18: note = Synthetic
  source 1..18: mol_type = other DNA
aggcccttgccaatgaat

SEQ ID NO: 420 - DNA, 40 bp, synthetic construct; identical to SEQ ID NO: 248
  misc_feature 1..40: note = Synthetic
  source 1..40: mol_type = other DNA

SEQ ID NO: 421 - DNA, 40 bp, synthetic construct; identical to SEQ ID NO: 249
  misc_feature 1..40: note = Synthetic
  source 1..40: mol_type = other DNA

SEQ ID NO: 422 - DNA, 19 bp, synthetic construct
  misc_feature 1..19: note = Synthetic
  source 1..19: mol_type = other DNA
aatgttgcaaggcgatcag

SEQ ID NO: 423 - DNA, 20 bp, synthetic construct
  misc_feature 1..20: note = Synthetic
  source 1..20: mol_type = other DNA
agccctcccgtatcgtagtt

SEQ ID NO: 424 - DNA, 40 bp, synthetic construct; identical to SEQ ID NO: 232
  misc_feature 1..40: note = Synthetic
  source 1..40: mol_type = other DNA

SEQ ID NO: 425 - DNA, 40 bp, synthetic construct; identical to SEQ ID NO: 233
  misc_feature 1..40: note = Synthetic
  source 1..40: mol_type = other DNA

SEQ ID NO: 426 - DNA, 20 bp, synthetic construct
  misc_feature 1..20: note = Synthetic
  source 1..20: mol_type = other DNA
atcgaagagaagcaggacga

SEQ ID NO: 427 - DNA, 20 bp, synthetic construct
  misc_feature 1..20: note = Synthetic
  source 1..20: mol_type = other DNA
tgctggtccatgaagatgaa

SEQ ID NO: 428 - DNA, 20 bp, synthetic construct
  misc_feature 1..20: note = Synthetic
  source 1..20: mol_type = other DNA
tcgagctgatgtttgacgac

SEQ ID NO: 429 - DNA, 20 bp, synthetic construct
  misc_feature 1..20: note = Synthetic
  source 1..20: mol_type = other DNA
ggacttgaggttgctctgct

SEQ ID NO: 430 - DNA, 40 bp, synthetic construct; identical to SEQ ID NO: 258
  misc_feature 1..40: note = Synthetic
  source 1..40: mol_type = other DNA

SEQ ID NO: 431 - DNA, 25 bp, synthetic construct
  misc_feature 1..25: note = Synthetic
  source 1..25: mol_type = other DNA
atcggcgtgaagcccaacagggcca

SEQ ID NO: 432 - DNA, 20 bp, synthetic construct
  misc_feature 1..20: note = Synthetic
  source 1..20: mol_type = other DNA
gtggacattggtttcagcaa

SEQ ID NO: 433 - DNA, 20 bp, synthetic construct
  misc_feature 1..20: note = Synthetic
  source 1..20: mol_type = other DNA
agctcatgcatcacaacagc

SEQ ID NO: 434 - DNA, 20 bp, synthetic construct
  misc_feature 1..20: note = Synthetic
  source 1..20: mol_type = other DNA
gagcaatggatagccgatgt

SEQ ID NO: 435 - DNA, 20 bp, synthetic construct
  misc_feature 1..20: note = Synthetic
  source 1..20: mol_type = other DNA
aagcgatgaatgatcccaag

SEQ ID NO: 436 - DNA, 20 bp, synthetic construct
  misc_feature 1..20: note = Synthetic
  source 1..20: mol_type = other DNA
tgtaacgcttcccggtagtc

SEQ ID NO: 437 - DNA, 20 bp, synthetic construct
  misc_feature 1..20: note = Synthetic
  source 1..20: mol_type = other DNA
cattgcaaagcgactgatgt

SEQ ID NO: 438 - DNA, 20 bp, synthetic construct
  misc_feature 1..20: note = Synthetic
  source 1..20: mol_type = other DNA
gatccgctccttgaactctg

SEQ ID NO: 439 - DNA, 60 bp, synthetic construct
  misc_feature 1..60: note = Synthetic
  source 1..60: mol_type = other DNA
gcagcccacctatcaaggtgtactgccttccagacgaacgaagagcgattgaggaaaagg

SEQ ID NO: 440 - DNA, 60 bp, synthetic construct
  misc_feature 1..60: note = Synthetic
  source 1..60: mol_type = other DNA
taggccgacaggctcatgccggccgccgccgccttttcctcaatcgctcttcgttcgtct

SEQ ID NO: 441 - DNA, 18 bp, synthetic construct
  misc_feature 1..18: note = Synthetic
  source 1..18: mol_type = other DNA
aggcccttgccaatgaat

SEQ ID NO: 442 - DNA, 40 bp, synthetic construct; identical to SEQ ID NO: 248
  misc_feature 1..40: note = Synthetic
  source 1..40: mol_type = other DNA

SEQ ID NO: 443 - DNA, 40 bp, synthetic construct; identical to SEQ ID NO: 249
  misc_feature 1..40: note = Synthetic
  source 1..40: mol_type = other DNA

SEQ ID NO: 444 - DNA, 19 bp, synthetic construct
  misc_feature 1..19: note = Synthetic
  source 1..19: mol_type = other DNA
aatgttgcaaggcgatcag

SEQ ID NO: 445 - DNA, 20 bp, synthetic construct
  misc_feature 1..20: note = Synthetic
  source 1..20: mol_type = other DNA
agccctcccgtatcgtagtt

SEQ ID NO: 446 - DNA, 20 bp, synthetic construct
  misc_feature 1..20: note = Synthetic
  source 1..20: mol_type = other DNA
gtttcagcaggccgcccagg

SEQ ID NO: 447 - DNA, 20 bp, synthetic construct
  misc_feature 1..20: note = Synthetic
  source 1..20: mol_type = other DNA
cgctgcataaccctgcttcg

SEQ ID NO: 448 - DNA, 40 bp, synthetic construct; identical to SEQ ID NO: 232
  misc_feature 1..40: note = Synthetic
  source 1..40: mol_type = other DNA

SEQ ID NO: 449 - DNA, 40 bp, synthetic construct; identical to SEQ ID NO: 233
  misc_feature 1..40: note = Synthetic
  source 1..40: mol_type = other DNA

SEQ ID NO: 450 - DNA, 20 bp, synthetic construct
  misc_feature 1..20: note = Synthetic
  source 1..20: mol_type = other DNA
atcgaagagaagcaggacga

SEQ ID NO: 451 - DNA, 20 bp, synthetic construct
  misc_feature 1..20: note = Synthetic
  source 1..20: mol_type = other DNA
tgctggtccatgaagatgaa

SEQ ID NO: 452 - DNA, 20 bp, synthetic construct
  misc_feature 1..20: note = Synthetic
  source 1..20: mol_type = other DNA
tcgagctgatgtttgacgac

SEQ ID NO: 453 - DNA, 25 bp, synthetic construct
  misc_feature 1..25: note = Synthetic
  source 1..25: mol_type = other DNA
atcggcgtgaagcccaacagggcca

SEQ ID NO: 454 - DNA, 20 bp, synthetic construct
  misc_feature 1..20: note = Synthetic
  source 1..20: mol_type = other DNA
gtggacattggtttcagcaa

SEQ ID NO: 455 - DNA, 60 bp, synthetic construct
  misc_feature 1..60: note = Synthetic
  source 1..60: mol_type = other DNA
caagcattgggttccgtatctaaccatgaccgtgcttcgaaccactcggagggacggttt

SEQ ID NO: 456 - DNA, 60 bp, synthetic construct
  misc_feature 1..60: note = Synthetic
  source 1..60: mol_type = other DNA
aaaccgtccctccgagtggttcgaagcacggtcatggttagatacggaacccaatgcttg

SEQ ID NO: 457 - DNA, 60 bp, synthetic construct
  misc_feature 1..60: note = Synthetic
  source 1..60: mol_type = other DNA
aggagaactagagcgtaattaccctgttatccctacaaacaccctttcaatgggcttcga

SEQ ID NO: 458 - DNA, 60 bp, synthetic construct
  misc_feature 1..60: note = Synthetic
  source 1..60: mol_type = other DNA
cattgaaagggtgtttgtagggataacagggtaattacgctctagttctccttagtaggt

SEQ ID NO: 459 - DNA, 20 bp, synthetic construct
  misc_feature 1..20: note = Synthetic
  source 1..20: mol_type = other DNA
aagcgatgaatgatcccaag

SEQ ID NO: 460 - DNA, 20 bp, synthetic construct
  misc_feature 1..20: note = Synthetic
  source 1..20: mol_type = other DNA
tgtaacgcttcccggtagtc

SEQ ID NO: 461 - DNA, 18 bp, synthetic construct
  misc_feature 1..18: note = Synthetic
  source 1..18: mol_type = other DNA
aggcccttgccaatgaat

SEQ ID NO: 462 - DNA, 40 bp, synthetic construct; identical to SEQ ID NO: 248
  misc_feature 1..40: note = Synthetic
  source 1..40: mol_type = other DNA

SEQ ID NO: 463 - DNA, 40 bp, synthetic construct; identical to SEQ ID NO: 249
  misc_feature 1..40: note = Synthetic
  source 1..40: mol_type = other DNA

SEQ ID NO: 464 - DNA, 19 bp, synthetic construct
  misc_feature 1..19: note = Synthetic
  source 1..19: mol_type = other DNA
aatgttgcaaggcgatcag

SEQ ID NO: 465 - DNA, 20 bp, synthetic construct
  misc_feature 1..20: note = Synthetic
  source 1..20: mol_type = other DNA
agccctcccgtatcgtagtt

SEQ ID NO: 466 - DNA, 40 bp, synthetic construct; identical to SEQ ID NO: 232
  misc_feature 1..40: note = Synthetic
  source 1..40: mol_type = other DNA

SEQ ID NO: 467 - DNA, 60 bp, synthetic construct
  misc_feature 1..60: note = Synthetic
  source 1..60: mol_type = other DNA
taattagcatttttcgctttattctgttgtcgagatcttctccttgcaggttcaacaact

SEQ ID NO: 468 - DNA, 59 bp, synthetic construct
  misc_feature 1..59: note = Synthetic
  source 1..59: mol_type = other DNA
aagatctcgacaacagaataaagcgaaaaatgctaataatgcactaacactcaggcctc

SEQ ID NO: 469 - DNA, 20 bp, synthetic construct
  misc_feature 1..20: note = Synthetic
  source 1..20: mol_type = other DNA
tgctggtccatgaagatgaa

SEQ ID NO: 470 - DNA, 20 bp, synthetic construct
  misc_feature 1..20: note = Synthetic
  source 1..20: mol_type = other DNA
tcgagctgatgtttgacgac

SEQ ID NO: 471 - DNA, 20 bp, synthetic construct
  misc_feature 1..20: note = Synthetic
  source 1..20: mol_type = other DNA
ggacttgaggttgctctgct

SEQ ID NO: 472 - DNA, 40 bp, synthetic construct; identical to SEQ ID NO: 258
  misc_feature 1..40: note = Synthetic
  source 1..40: mol_type = other DNA

SEQ ID NO: 473 - DNA, 25 bp, synthetic construct
  misc_feature 1..25: note = Synthetic
  source 1..25: mol_type = other DNA
atcggcgtgaagcccaacagggcca

SEQ ID NO: 474 - DNA, 20 bp, synthetic construct
  misc_feature 1..20: note = Synthetic
  source 1..20: mol_type = other DNA
gtggacattggtttcagcaa

SEQ ID NO: 475 - DNA, 60 bp, synthetic construct
  misc_feature 1..60: note = Synthetic
  source 1..60: mol_type = other DNA
caatgtctgatgcaatatggacaattggtttcttggtctcattaccctgttatccctaca

SEQ ID NO: 476 - DNA, 60 bp, synthetic construct
  misc_feature 1..60: note = Synthetic
  source 1..60: mol_type = other DNA
accttcgggtgggcctttctgcgtttataggtctcatgcttacgctctagttctccttag

SEQ ID NO: 477 - DNA, 60 bp, synthetic construct
  misc_feature 1..60: note = Synthetic
  source 1..60: mol_type = other DNA
cgaattgaaacggagggcgacaagaagggcgagaagtcgggcttctacgtcggccacctc

SEQ ID NO: 478 - DNA, 59 bp, synthetic construct
  misc_feature 1..59: note = Synthetic
  source 1..59: mol_type = other DNA
cgacttctcgcccttcttgtcgccctccgtttcaattcggtgcttcttgccgtccatga

SEQ ID NO: 479 - DNA, 20 bp, synthetic construct
  misc_feature 1..20: note = Synthetic
  source 1..20: mol_type = other DNA
cattgcaaagcgactgatgt

SEQ ID NO: 480 - DNA, 20 bp, synthetic construct
  misc_feature 1..20: note = Synthetic
  source 1..20: mol_type = other DNA
gatccgctccttgaactctg

SEQ ID NO: 481 - DNA, 18 bp, synthetic construct
  misc_feature 1..18: note = Synthetic
  source 1..18: mol_type = other DNA
aggcccttgccaatgaat

SEQ ID NO: 482 - DNA, 19 bp, synthetic construct
  misc_feature 1..19: note = Synthetic
  source 1..19: mol_type = other DNA
aatgttgcaaggcgatcag

SEQ ID NO: 483 - DNA, 20 bp, synthetic construct
  misc_feature 1..20: note = Synthetic
  source 1..20: mol_type = other DNA
agccctcccgtatcgtagtt

SEQ ID NO: 484 - DNA, 60 bp, synthetic construct
  misc_feature 1..60: note = Synthetic
  source 1..60: mol_type = other DNA
attgaaagggtgtttgtagggataacagggtaatgagaccaagaaaccaattgtccatat

SEQ ID NO: 485 - DNA, 60 bp, synthetic construct
  misc_feature 1..60: note = Synthetic
  source 1..60: mol_type = other DNA
gagaacctactaaggagaactagagcgtaagcatgagacctataaacgcagaaaggccca

SEQ ID NO: 486 - DNA, 22 bp, synthetic construct
  misc_feature 1..22: note = Synthetic
  source 1..22: mol_type = other DNA
ggtctcagtaatatcaagcttg

SEQ ID NO: 487 - DNA, 24 bp, synthetic construct
  misc_feature 1..24: note = Synthetic
  source 1..24: mol_type = other DNA
taggtctcaagcaactggatggcg

SEQ ID NO: 488 - DNA, 60 bp, synthetic construct
  misc_feature 1..60: note = Synthetic
  source 1..60: mol_type = other DNA
ggtctcagtaaggcgcgcccttggcagaacatatccatcgcgtccgccatctccagcagc

SEQ ID NO: 489 - DNA, 60 bp, synthetic construct
  misc_feature 1..60: note = Synthetic
  source 1..60: mol_type = other DNA
ggtctcagatttatcttcgtttcctgcaggtttttgttctgtgcagttgggttaagaata

SEQ ID NO: 490 - DNA, 60 bp, synthetic construct
  misc_feature 1..60: note = Synthetic
  source 1..60: mol_type = other DNA
ggtctcagtaaggtaatgcttggcatgttcatatagatggtttaacagatcattataaag

SEQ ID NO: 491 - DNA, 60 bp, synthetic construct
  misc_feature 1..60: note = Synthetic
  source 1..60: mol_type = other DNA
ggtctcatagtgcattaaatcctggaggcgttaatttacttctatgtgcttcaaaggctt

SEQ ID NO: 492 - DNA, 60 bp, synthetic construct
  misc_feature 1..60: note = Synthetic
  source 1..60: mol_type = other DNA
ggtctcaactagtatgttctagcgcttgcaccatcccatttaactgtaagaagaattgca

SEQ ID NO: 493 - DNA, 60 bp, synthetic construct
  misc_feature 1..60: note = Synthetic
  source 1..60: mol_type = other DNA
ggtctcactaaacatataatatagcaacaaaaagaatgaagcaatcgatgttagtacatg

SEQ ID NO: 494 - DNA, 60 bp, synthetic construct
  misc_feature 1..60: note = Synthetic
  source 1..60: mol_type = other DNA
ggtctcattagtctaatgaaggtttacttcaatattacgcttcattaaatggtttaactg

SEQ ID NO: 495 - DNA, 60 bp, synthetic construct
  misc_feature 1..60: note = Synthetic
  source 1..60: mol_type = other DNA
ggtctcaagcaggaccataagaagtccgaaaaactattaatctgtccaaaatgtttaata

SEQ ID NO: 496 - DNA, 20 bp, synthetic construct
  misc_feature 1..20: note = Synthetic
  source 1..20: mol_type = other DNA
tccagttgatccaccattga

SEQ ID NO: 497 - DNA, 20 bp, synthetic construct
  misc_feature 1..20: note = Synthetic
  source 1..20: mol_type = other DNA
caaccacaaatgaccagcac

SEQ ID NO: 498 - DNA, 40 bp, synthetic construct; identical to SEQ ID NO: 232
  misc_feature 1..40: note = Synthetic
  source 1..40: mol_type = other DNA

SEQ ID NO: 499 - DNA, 60 bp, synthetic construct
  misc_feature 1..60: note = Synthetic
  source 1..60: mol_type = other DNA
atatggacaattggtttcttggtctcattacatatatacacatgtatatatatcgtatgc

SEQ ID NO: 500 - DNA, 60 bp, synthetic construct
  misc_feature 1..60: note = Synthetic
  source 1..60: mol_type = other DNA
tgggcctttctgcgtttataggtctcatgctgtatacctatgaatgtcagtaagtatgta

SEQ ID NO: 501 - DNA, 40 bp, synthetic construct; identical to SEQ ID NO: 233
  misc_feature 1..40: note = Synthetic
  source 1..40: mol_type = other DNA

SEQ ID NO: 502 - DNA, 20 bp, synthetic construct
  misc_feature 1..20: note = Synthetic
  source 1..20: mol_type = other DNA
taggagtgcggttggaacgt

SEQ ID NO: 503 - DNA, 20 bp, synthetic construct
  misc_feature 1..20: note = Synthetic
  source 1..20: mol_type = other DNA
tgctggtccatgaagatgaa

SEQ ID NO: 504 - DNA, 20 bp, synthetic construct
  misc_feature 1..20: note = Synthetic
  source 1..20: mol_type = other DNA
tcgagctgatgtttgacgac

SEQ ID NO: 505 - DNA, 20 bp, synthetic construct
  misc_feature 1..20: note = Synthetic
  source 1..20: mol_type = other DNA
ggacttgaggttgctctgct

SEQ ID NO: 506 - DNA, 40 bp, synthetic construct; identical to SEQ ID NO: 258
  misc_feature 1..40: note = Synthetic
  source 1..40: mol_type = other DNA

SEQ ID NO: 507 - DNA, 25 bp, synthetic construct
  misc_feature 1..25: note = Synthetic
  source 1..25: mol_type = other DNA
atcggcgtgaagcccaacagggcca

SEQ ID NO: 508 - DNA, 20 bp, synthetic construct
  misc_feature 1..20: note = Synthetic
  source 1..20: mol_type = other DNA
gtggacattggtttcagcaa

SEQ ID NO: 509 - DNA, 60 bp, synthetic construct; identical to SEQ ID NO: 457
  misc_feature 1..60: note = Synthetic
  source 1..60: mol_type = other DNA

SEQ ID NO: 510 - DNA, 60 bp, synthetic construct; identical to SEQ ID NO: 458
  misc_feature 1..60: note = Synthetic
  source 1..60: mol_type = other DNA

SEQ ID NO: 511 - DNA, 20 bp, synthetic construct
  misc_feature 1..20: note = Synthetic
  source 1..20: mol_type = other DNA
aagcgatgaatgatcccaag

SEQ ID NO: 512 - DNA, 20 bp, synthetic construct
  misc_feature 1..20: note = Synthetic
  source 1..20: mol_type = other DNA
tgtaacgcttcccggtagtc

SEQ ID NO: 513 - DNA, 20 bp, synthetic construct
  misc_feature 1..20: note = Synthetic
  source 1..20: mol_type = other DNA
cattgcaaagcgactgatgt

SEQ ID NO: 514 - DNA, 20 bp, synthetic construct
  misc_feature 1..20: note = Synthetic
  source 1..20: mol_type = other DNA
gatccgctccttgaactctg

SEQ ID NO: 515 - DNA, 18 bp, synthetic construct
  misc_feature 1..18: note = Synthetic
  source 1..18: mol_type = other DNA
aggcccttgccaatgaat

SEQ ID NO: 516 - DNA, 40 bp, synthetic construct; identical to SEQ ID NO: 248
  misc_feature 1..40: note = Synthetic
  source 1..40: mol_type = other DNA

SEQ ID NO: 517 - DNA, 40 bp, synthetic construct; identical to SEQ ID NO: 249
  misc_feature 1..40: note = Synthetic
  source 1..40: mol_type = other DNA

SEQ ID NO: 518 - DNA, 19 bp, synthetic construct
  misc_feature 1..19: note = Synthetic
  source 1..19: mol_type = other DNA
aatgttgcaaggcgatcag

SEQ ID NO: 519 - DNA, 20 bp, synthetic construct
  misc_feature 1..20: note = Synthetic
  source 1..20: mol_type = other DNA
agccctcccgtatcgtagtt

SEQ ID NO: 520 - DNA, 60 bp, synthetic construct
  misc_feature 1..60: note = Synthetic
  source 1..60: mol_type = other DNA
gcatacgatatatatacatgtgtatatatgtaatgagaccaagaaaccaattgtccatat

SEQ ID NO: 521 - DNA, 60 bp, synthetic construct
  misc_feature 1..60: note = Synthetic
  source 1..60: mol_type = other DNA
tacatacttactgacattcataggtatacagcatgagacctataaacgcagaaaggccca

SEQ ID NO: 522 - DNA, 40 bp, synthetic construct; identical to SEQ ID NO: 232
  misc_feature 1..40: note = Synthetic
  source 1..40: mol_type = other DNA

SEQ ID NO: 523 - DNA, 60 bp, synthetic construct
  misc_feature 1..60: note = Synthetic
  source 1..60: mol_type = other DNA
gggagtatctggctgggccaacgttccaaccgcactcctaccggccagcctcgcagagca

SEQ ID NO: 524 - DNA, 60 bp, synthetic construct
  misc_feature 1..60: note = Synthetic
  source 1..60: mol_type = other DNA
gcggtgctcaacgggaatcctgctctgcgaggctggccggtaggagtgcggttggaacgt

SEQ ID NO: 525 - DNA, 20 bp, synthetic construct
  misc_feature 1..20: note = Synthetic
  source 1..20: mol_type = other DNA
tgctggtccatgaagatgaa

SEQ ID NO: 526 - DNA, 20 bp, synthetic construct
  misc_feature 1..20: note = Synthetic
  source 1..20: mol_type = other DNA
tcgagctgatgtttgacgac

SEQ ID NO: 527 - DNA, 20 bp, synthetic construct
  misc_feature 1..20: note = Synthetic
  source 1..20: mol_type = other DNA
ggacttgaggttgctctgct

SEQ ID NO: 528 - DNA, 40 bp, synthetic construct; identical to SEQ ID NO: 258
  misc_feature 1..40: note = Synthetic
  source 1..40: mol_type = other DNA

SEQ ID NO: 529 - DNA, 25 bp, synthetic construct
  misc_feature 1..25: note = Synthetic
  source 1..25: mol_type = other DNA
atcggcgtgaagcccaacagggcca

SEQ ID NO: 530 - DNA, 20 bp, synthetic construct
  misc_feature 1..20: note = Synthetic
  source 1..20: mol_type = other DNA
gtggacattggtttcagcaa

SEQ ID NO: 531 - DNA, 20 bp, synthetic construct
  misc_feature 1..20: note = Synthetic
  source 1..20: mol_type = other DNA
agctcatgcatcacaacagc

SEQ ID NO: 532 - DNA, 20 bp, synthetic construct
  misc_feature 1..20: note = Synthetic
  source 1..20: mol_type = other DNA
gagcaatggatagccgatgt

SEQ ID NO: 533 - DNA, 20 bp, synthetic construct
  misc_feature 1..20: note = Synthetic
  source 1..20: mol_type = other DNA
aagcgatgaatgatcccaag

SEQ ID NO: 534 - DNA, 20 bp, synthetic construct
  misc_feature 1..20: note = Synthetic
  source 1..20: mol_type = other DNA
tgtaacgcttcccggtagtc

SEQ ID NO: 535 - DNA, 20 bp, synthetic construct
  misc_feature 1..20: note = Synthetic
  source 1..20: mol_type = other DNA
cattgcaaagcgactgatgt

SEQ ID NO: 536 - DNA, 20 bp, synthetic construct
  misc_feature 1..20: note = Synthetic
  source 1..20: mol_type = other DNA
gatccgctccttgaactctg

SEQ ID NO: 537 - DNA, 18 bp, synthetic construct
  misc_feature 1..18: note = Synthetic
  source 1..18: mol_type = other DNA
aggcccttgccaatgaat

SEQ ID NO: 538 - DNA, 40 bp, synthetic construct; identical to SEQ ID NO: 248
  misc_feature 1..40: note = Synthetic
  source 1..40: mol_type = other DNA

SEQ ID NO: 539 - DNA, 40 bp, synthetic construct; identical to SEQ ID NO: 249
  misc_feature 1..40: note = Synthetic
  source 1..40: mol_type = other DNA

SEQ ID NO: 540 - DNA, 19 bp, synthetic construct
  misc_feature 1..19: note = Synthetic
  source 1..19: mol_type = other DNA
aatgttgcaaggcgatcag

SEQ ID NO: 541 - DNA, 20 bp, synthetic construct
  misc_feature 1..20: note = Synthetic
  source 1..20: mol_type = other DNA
agccctcccgtatcgtagtt

SEQ ID NO: 542 - DNA, 40 bp, synthetic construct; identical to SEQ ID NO: 232
  misc_feature 1..40: note = Synthetic
  source 1..40: mol_type = other DNA

SEQ ID NO: 543 - DNA, 60 bp, synthetic construct
  misc_feature 1..60: note = Synthetic
  source 1..60: mol_type = other DNA
cacgcgacaagcacgagcgagatatcccaatcaagctagtccggccagcctcgcagagca

SEQ ID NO: 544 - DNA, 60 bp, synthetic construct; identical to SEQ ID NO: 524
  misc_feature 1..60: note = Synthetic
  source 1..60: mol_type = other DNA

SEQ ID NO: 545 - DNA, 20 bp, synthetic construct
  misc_feature 1..20: note = Synthetic
  source 1..20: mol_type = other DNA
tgctggtccatgaagatgaa

SEQ ID NO: 546 - DNA, 20 bp, synthetic construct
  misc_feature 1..20: note = Synthetic
  source 1..20: mol_type = other DNA
tcgagctgatgtttgacgac

SEQ ID NO: 547 - DNA, 20 bp, synthetic construct
  misc_feature 1..20: note = Synthetic
  source 1..20: mol_type = other DNA
ggacttgaggttgctctgct

SEQ ID NO: 548 - DNA, 40 bp, synthetic construct; identical to SEQ ID NO: 258
  misc_feature 1..40: note = Synthetic
  source 1..40: mol_type = other DNA

SEQ ID NO: 549 - DNA, 25 bp, synthetic construct
  misc_feature 1..25: note = Synthetic
  source 1..25: mol_type = other DNA
atcggcgtgaagcccaacagggcca

SEQ ID NO: 550 - DNA, 20 bp, synthetic construct
  misc_feature 1..20: note = Synthetic
  source 1..20: mol_type = other DNA
gtggacattggtttcagcaa

SEQ ID NO: 551 - DNA, 20 bp, synthetic construct
  misc_feature 1..20: note = Synthetic
  source 1..20: mol_type = other DNA
aagcgatgaatgatcccaag

SEQ ID NO: 552 - DNA, 20 bp, synthetic construct
  misc_feature 1..20: note = Synthetic
  source 1..20: mol_type = other DNA
tgtaacgcttcccggtagtc

SEQ ID NO: 553 - DNA, 20 bp, synthetic construct
  misc_feature 1..20: note = Synthetic
  source 1..20: mol_type = other DNA
cattgcaaagcgactgatgt

SEQ ID NO: 554 - DNA, 20 bp, synthetic construct
  misc_feature 1..20: note = Synthetic
  source 1..20: mol_type = other DNA
gatccgctccttgaactctg

SEQ ID NO: 555 - DNA, 18 bp, synthetic construct
  misc_feature 1..18: note = Synthetic
  source 1..18: mol_type = other DNA
aggcccttgccaatgaat

SEQ ID NO: 556 - DNA, 40 bp, synthetic construct; identical to SEQ ID NO: 248
  misc_feature 1..40: note = Synthetic
  source 1..40: mol_type = other DNA

SEQ ID NO: 557 - DNA, 20 bp, synthetic construct
  misc_feature 1..20: note = Synthetic
  source 1..20: mol_type = other DNA
agccctcccgtatcgtagtt

SEQ ID NO: 558 - DNA, 22 bp, synthetic construct
  misc_feature 1..22: note = Synthetic
  source 1..22: mol_type = other DNA
ctcttgccatcggatgtgccca

SEQ ID NO: 559 - DNA, 24 bp, synthetic construct
  misc_feature 1..24: note = Synthetic
  source 1..24: mol_type = other DNA
ccagtgtggctggtcatcctctca

SEQ ID NO: 560 - DNA, 20 bp, synthetic construct
  misc_feature 1..20: note = Synthetic
  source 1..20: mol_type = other DNA
ttgtcggcggtggtgatgtc

SEQ ID NO: 561 - DNA, 24 bp, synthetic construct
  misc_feature 1..24: note = Synthetic
  source 1..24: mol_type = other DNA
atgcggtgaactgtggaataaacg

SEQ ID NO: 562 - DNA, 21 bp, synthetic construct
  misc_feature 1..21: note = Synthetic
  source 1..21: mol_type = other DNA
acgacgcccgtgattttgtag

SEQ ID NO: 563 - DNA, 20 bp, synthetic construct
  misc_feature 1..20: note = Synthetic
  source 1..20: mol_type = other DNA
gccttccagacgaacgaaga

(19) United States
(12) Patent Application Publication
(10) Pub. No.: US 2026/0337621 A1
(43) Pub. Date: Oct. 29, 2026

(54) SUPERIOR CONJUGATIVE PLASMIDS DELIVERED BY BACTERIA TO EUKARYOTIC CELLS

(71) Applicants: THE UNIVERSITY OF WESTERN ONTARIO, London (CA); UNIVERSITY OF GUELPH, Guelph (CA)

(72) Inventors: Bogumil KARAS, London (CA); David EDGELL, London (CA); Gregory GLOOR, London (CA); Rebecca SHAPIRO, Guelph (CA)

(73) Assignees: THE UNIVERSITY OF WESTERN ONTARIO, London, ON (CA); UNIVERSITY OF GUELPH, Guelph, ON (CA)

(21) Appl. No.: 19/105,992

(22) PCT Filed: Aug. 23, 2023
(86) PCT No.: PCT/CA2023/051115

§ 371 Date: Feb. 24, 2025

Related U.S. Application Data
(60) Provisional application No. 63/400,610, filed on Aug. 24, 2022.
(60) Provisional application No. 63/428,386, filed on Nov. 28, 2022.

(51) Int. Cl. C12N 15/81

(52) U.S. Cl. CPC C12N 15/81

(57) ABSTRACT

A DNA fragment for facilitating the transfer of a plasmid transferable (“transferable plasmid”) by conjugation from a donor bacterium to a recipient eukaryotic cell, the DNA fragment comprising a mutated traJ promoter operatively linked to a traJ gene that enables transfer of the transferable plasmid by conjugation from the donor bacterium to the recipient eukaryotic cell. Also, bacteria comprising the DNA fragment and methods of conjugatively transferring a gene of interest from a donor bacterium into a recipient eukaryotic cell.

### CROSS REFERENCE TO RELATED APPLICATIONS

This application is a national stage application under 35 U.S.C. § 371 of International Application No. PCT/CA2023/051115, filed Aug. 23, 2023, which in turn claims the benefit under 35 U.S.C. § 119 (e) of U.S. Provisional Ser. No. 63/400,610, filed Aug. 24, 2022 and of U.S. Provisional Ser. No. 63/428,386, filed Nov. 28, 2022, the contents of each of which are hereby incorporated by reference in its entirety into the present disclosure.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 24, 2025, is named “118494.0198.xml” and is 716,264 bytes in size. The sequence listing contained in this .XML file is part of the specification and is hereby incorporated by reference herein in its entirety.

### FIELD

The present disclosure relates to conjugative plasmids, more particularly to conjugative plasmids delivered by bacteria to diverse recipient eukaryotic cells.

### BACKGROUND ART

The fungal kingdom is exquisitely diverse and home to countless species with profound impacts on ecological nutrient cycling, industrial manufacturing, and health and disease in humans, animals, and plants^{1,2}. Yeast species are amongst the best-studied fungi and include the common yeast Saccharomyces cerevisiae, which is a primary fermenter of beer, wine, and bread, and a ubiquitous eukaryotic model system. S. cerevisiae is also an important synthetic biology chassis for the production of insulin, vaccine components, and other critical recombinant proteins³. The closely-related Saccharomyces boulardii is a promising probiotic therapeutic, particularly in the context of obesity and type 2 diabetes^{4,5}. Yeasts are also critical components of the human microbiota, including Candida species associated with vaginal yeast infections and invasive candidiasis⁶, as well as Malassezia species with notable associations to Crohn's disease and pancreatic cancer^{7,8}. The skin-associated yeast Candida auris is an emerging fungal pathogen that can cause life-threatening infections and is highly refractory to antifungal drug treatment⁹⁻¹¹. These diverse and critical roles in health, disease, and industrial manufacturing highlight the importance of studying and manipulating the biology of these key yeast species.

Given the diversity of yeast species and the breadth of niches they inhabit, there is a need to develop improved and innovative methods for DNA transformation in these organisms. Genetic transformation techniques enable the manipulation of genomes of industrially important yeasts, and further promote the ability to target, modify, or damage the genomes of fungal pathogens. Indeed, genetic-editing tools such as CRISPR have a promising role as novel antimicrobial agents due to their ability to specifically target pathogen-associated genes, leading to microbial death, growth inhibition, or targeted deletion of genes involved in antimicrobial resistance or virulence¹²⁻¹⁸. However, laboratory-based transformation protocols typically rely on chemical strategies to promote DNA uptake, which is not broadly applicable for manipulating yeasts in their native environments, such as those inhabiting the microbiome. One innovative strategy to promote the uptake of genetic material in situ is to exploit bacterial conjugation as a viable mechanism to transfer plasmids from bacteria to a recipient microbe via the bacterial type IV secretion system. Previous work has demonstrated the utility of conjugation for transferring plasmids, including those encoding CRISPR-based antimicrobials, between bacterial species, both in vitro^{19,20} and in vivo in mouse microbiome models²¹⁻²³. While conjugation typically occurs between bacterial species, cross-kingdom conjugation from bacteria to yeast and algae has been demonstrated²⁴⁻²⁷. Despite recently optimized protocols^{28,29}, conjugation to yeast still suffers from relatively low conjugation frequency compared to prokaryotic recipients.

The pTA-Mob 2.0 plasmid²⁸ is an example of a conjugative plasmid and it is composed of genetic elements required for plasmid maintenance and transfer^{30,31}. Two regions, Tra1 and Tra2, are responsible for the transfer of plasmid DNA. Tra1 harbors the relaxase-(traH-J), primase-(traA-G), and leader-(traK-M) operons, which together coordinate mobilization of the plasmid to the recipient³¹. The relaxase and leader operon encode the relaxosome, a protein complex essential for initial DNA-processing during conjugation. Assembly of the protein complex (TraH-J) is initiated by TraJ binding to the 19-bp inverted repeat sequence in the origin of transfer (oriT)³²⁻³⁴. The interaction of TraI and TraJ, which is stabilized by TraH, then orients the relaxase toward the nic-site³⁴. After formation of the relaxosome, TraI nicks and covalently binds to the plasmid DNA, ready for transfer to the recipient cell^{35,36}. The efficiency of the nicking reaction is attenuated by the binding of Trak to the oriT which orients the plasmid DNA into a more favorable position^{33,37} TraC1, of the primase operon, is a DNA primase that co-transfers (along with single-stranded binding (SSB) proteins) with the DNA to the recipient cell where it is involved in the restoration of a double-stranded plasmid³⁸. The primase operon also includes the TraG protein, which couples DNA processing by the relaxosome to DNA transfer by delivering the protein-DNA complex to the mating pair formation proteins^{39,40}. The Tra2 region contains proteins (TrbB-L, and TraF) required for mating pair formation, many of which are associated with the cell membrane. TrbC encodes a peptide responsible for forming the pilus. This peptide undergoes maturation by proteolytic cleavage followed by cyclization by TraF resulting in rigid pili^{41,42}. The pilus allows initial contact between the two cells and enables the transfer of single stranded plasmid DNA to the recipient cell.

### SUMMARY

In one embodiment, the present disclosure relates to a DNA fragment for facilitating the transfer of a plasmid transferable (“transferable plasmid”) by conjugation from a donor bacterium to a recipient eukaryotic cell, the DNA fragment comprising a mutated traJ promoter operatively linked to a traJ gene that enables transfer of the transferable plasmid by conjugation from the donor bacterium to the recipient eukaryotic cell.

In one embodiment of the DNA fragment of the present disclosure, the transferable plasmid is self-transmissible.

In another embodiment of the DNA fragment of the present disclosure, the transferable plasmid is not self-transmissible.

In another embodiment of the DNA fragment of the present disclosure, the DNA fragment is included in the genome of the donor bacterium.

In another embodiment of the DNA fragment of the present disclosure, the DNA fragment is included in the transferable plasmid.

In another embodiment of the DNA fragment of the present disclosure, the DNA fragment is included in a non-transferable plasmid within the donor bacterium.

In another embodiment of the DNA fragment of the present disclosure, the DNA fragment further comprises a gene of interest.

In another embodiment of the DNA fragment of the present disclosure, the DNA fragment is included in the transferable plasmid and gene of interest can be expressed in the recipient eukaryotic cell.

In another embodiment of the DNA fragment of the present disclosure, the gene of interest encodes for a protein that kills the recipient eukaryotic cell.

In another embodiment of the DNA fragment of the present disclosure, expression of the gene of interest is inducible.

In another embodiment of the DNA fragment of the present disclosure, the recipient eukaryotic cell is a fungal cell. In one aspect, the fungal cell is a yeast cell. In aspects, the yeast cell is any one of Saccharomyces cerevisiae, Candida auris, Candida bromeliacearum, Candida tolerans, Metschnikowia gruessii, Metschnikowia borealis, Metschnikowia pulcherrima and Metschnikowia lunata.

In another embodiment of the DNA fragment of the present disclosure, the recipient eukaryotic cell is an animal kingdom cell or a plant kingdom cell.

In another embodiment of the DNA fragment of the present disclosure, the recipient eukaryotic cell is a nematode cell.

In another embodiment of the DNA fragment of the present disclosure, the recipient eukaryotic cell is an algae cell.

In another embodiment of the DNA fragment of the present disclosure, the donor bacterium is a gram-negative bacterium.

In another embodiment of the DNA fragment of the present disclosure, the gram-negative bacterium is E. coli.

In another embodiment of the DNA fragment of the present disclosure, the donor bacterium is a probiotic.

In another embodiment of the DNA fragment of the present disclosure, the traJ promoter is a traJ promoter of a pTA-Mob 2.0 plasmid.

The DNA fragment according to any one of claims 1 to 20, wherein the mutated traJ promoter includes the nucleotide sequence ccgag (SEQ ID NO: 3).

The DNA fragment according to any one of claims 1 to 20, wherein the mutated traJ promoter comprises, or consists essentially of, or consists of SEQ ID NO: 2.

In another embodiment of the DNA fragment of the present disclosure, at least one of (i) the DNA fragment, (ii) the donor bacterium, (iii) the transferable plasmid and (iv) a non-transferable plasmid within the donor bacterium, includes further mutations that in combination with the mutated traJ promoter improves the transfer of the transferable plasmid from the donor bacterium to the recipient eukaryotic cell.

In another embodiment, the present invention provides for a bacterium for facilitating the transfer of a plasmid transferable (“transferable plasmid”) by conjugation from the bacterium to a recipient eukaryotic cell, the bacterium comprising: (i) a transferable plasmid and (ii) a DNA fragment carrying a mutated traJ promoter operatively linked to a traJ gene that enables transfer of the transferable plasmid by conjugation from the donor bacterium to the recipient eukaryotic cell.

In one embodiment of the bacterium of the present disclosure, the transferable plasmid is self-transmissible.

In another embodiment of the bacterium of the present disclosure, the transferable plasmid is not self-transmissible.

In another embodiment of the bacterium of the present disclosure, the DNA fragment is included in the genome of the bacterium.

In another embodiment of the bacterium of the present disclosure, the DNA fragment is included in the transferable plasmid.

In another embodiment of the bacterium of the present disclosure, the bacterium further includes a non-transferable plasmid and the DNA fragment is included in the non-transferable plasmid.

In another embodiment of the bacterium of the present disclosure, the DNA fragment further carries a gene of interest.

In another embodiment of the bacterium of the present disclosure, the DNA fragment is included in the transferable plasmid and the gene of interest is expressed in the recipient eukaryotic cell.

In another embodiment of the bacterium of the present disclosure, the gene of interest encodes for a protein that kills the recipient eukaryotic cell.

In another embodiment of the bacterium of the present disclosure, expression of the gene of interest is inducible.

In another embodiment of the bacterium of the present disclosure, the recipient eukaryotic cell is a fungal cell.

In another embodiment of the bacterium of the present disclosure, the fungal cell is a yeast cell.

In another embodiment of the bacterium of the present disclosure, the yeast cell is of any one of Saccharomyces cerevisiae, Candida auris, Candida bromeliacearum, Candida tolerans, Metschnikowia gruessii, Metschnikowia borealis, Metschnikowia pulcherrima and Metschnikowia lunata.

In another embodiment of the bacterium of the present disclosure, the recipient eukaryotic cell is an animal kingdom cell or a plant kingdom cell.

In another embodiment of the bacterium of the present disclosure, the recipient eukaryotic cell is a nematode cell.

In another embodiment of the bacterium of the present disclosure, the recipient eukaryotic cell is an algae cell.

In another embodiment of the bacterium of the present disclosure, the bacterium is a gram-negative bacterium.

In another embodiment of the bacterium of the present disclosure, the gram-negative bacterium is E. coli.

In another embodiment of the bacterium of the present disclosure, the bacterium is a probiotic.

In another embodiment of the bacterium of the present disclosure, the traJ promoter is of a pTA-Mob 2.0 plasmid.

In another embodiment of the bacterium of the present disclosure, the mutated traJ promoter includes the nucleotide sequence ccgag (SEQ ID NO: 3).

In another embodiment of the bacterium of the present disclosure, the mutated traJ promoter comprises, or consists essentially of, or consists of SEQ ID NO: 2.

In another embodiment of the bacterium of the present disclosure, at least one of the DNA fragment, the bacterium and the transferable plasmid, includes further mutations that in combination with the mutated traJ improves the transfer of the transferable plasmid from the donor bacterium to the recipient eukaryotic cell.

In another embodiment, the present disclosure relates to a method of conjugatively transferring a gene of interest from a donor bacterium into a recipient eukaryotic cell, the method comprising: (a) providing the bacterium of claim 28, the transferable plasmid including a gene of interest, and (b) conjugatively transferring the transferable plasmid from the bacterium to the recipient eukaryotic cell, thereby transferring the gene of interest to the recipient eukaryotic cell.

In one embodiment of the method of conjugatively transferring a gene of interest from a donor bacterium into a recipient eukaryotic cell, the gene of interest is operably joined to a promoter functional in the recipient eukaryotic cell.

In another embodiment of the method of conjugatively transferring a gene of interest from a donor bacterium into a recipient eukaryotic cell, the gene of interest is not native to the donor bacterium.

In another embodiment of the method of conjugatively transferring a gene of interest from a donor bacterium into a recipient eukaryotic cell, the gene of interest encodes a protein that kills the recipient cell.

In another embodiment of the method of conjugatively transferring a gene of interest from a donor bacterium into a recipient eukaryotic cell, the recipient eukaryotic cell is a fungal cell, and the gene of interest encodes an antifungal protein that kills the fungal cell.

In another embodiment of the method of conjugatively transferring a gene of interest from a donor bacterium into a recipient eukaryotic cell, expression of the gene of interest is inducible.

In another embodiment of the method of conjugatively transferring a gene of interest from a donor bacterium into recipient eukaryotic cell, the mutated traJ promoter includes the nucleotide sequence ccgag (SEQ ID NO: 3).

In another embodiment of the method of conjugatively transferring a gene of interest from a donor bacterium into a recipient eukaryotic cell, the mutated traJ promoter comprises, or consists essentially of, or consists of SEQ ID NO: 2.

### BRIEF DESCRIPTION OF THE DRAWINGS

The present disclosure will become more fully understood from the detailed description given herein and from the accompanying drawings, which are given by way of illustration only and do not limit the intended scope of the disclosure.

FIG. 1: Experiment Design. The eight-step flowchart shows experiments and major findings described in this disclosure.

FIG. 2: Transfer of minimized plasmids from E. coli to S. cerevisiae via conjugation. The pTA-Mob 2.0 plasmid was used as a template to create versions M1-M4. M3C1 was used as a template to create M5-M8. When available, two clones of the same plasmid were tested (e.g., M1 C1 and C2). Error bars represent the +95% confidence interval (Student's t-test was used to carry out pairwise comparisons between pTA-Mob 2.0 (control) and minimized versions of pTA-Mob 2.0: *P<0.05, **P<0.01; ***P<0.001). N=9 for all strains except N=27 for pTA-Mob 2.0.

FIG. 3: Identification of mutations in M3C1 responsible for improved conjugation to S. cerevisiae. S. cerevisiae transconjugant colony formation on minimal plates lacking histidine following conjugation of control: pTA-Mob 2.0, M3C1; hybrid: M3C1/M3C2; or mutated pTA-Mob 2.0 Tp/To plasmids from E. coli. Plasmid schematics for M3C1_F1-F5 generated by swapping PCR-amplified fragments from M3C1 into M3C2 are shown. Green arrows represent fragments originating from M3C1 and grey arrows represent fragments originating from M3C2. Mutations in pTA-Mob 2.0 Tp/To plasmids are indicated by green stars in the schematic of traJ (Tp, a cluster of mutations; and To, a single mutation). Transconjugant colony counts are reported below the plate images.

FIGS. 4A-4C: Creation and analysis of the pSC5 conjugative plasmid. 4A) Schematic of pSC5 plasmid map. N-Nourseothricin resistance gene encoded with the standard code for diatom (green) or the alternative yeast nuclear code for Candidal Metschnikowia (orange; Note—Also correctly translated in S. cerevisiae), HCA-HIS3, CEN6 and ARS4 for selection, replication, and maintenance in S. cerevisiae. 4B) Conjugation frequency of pSC5 (grey) compared to pTA-Mob 2.0 (black) in either a cis or trans setup from E. coli to S. cerevisiae. 4C) Bacterial conjugation frequency of pSC5 from E. coli to E. coli in a cis-configuration. Error bars represent the +95% confidence interval (Student's t-test: *P<0.05, **P<0.01, ***P<0.001). N=3 for cis- and N=4 for trans-experiment in S. cerevisiae and N=3 for cis-experiment in E. coli.

FIGS. 5A to 5C: Conjugation of pSC5 to wild yeast strains. 5A) pSC5 conjugated to S. cerevisiae and wild yeasts M. gruessi and C. auris were plated on YPD plates supplemented with NTC (100 μg mL⁻¹). 5B, 5C) Diagnostic double restriction enzyme digestion (EcoRI-HF and Agel-HF) of pSC5 plasmid rescued from yeast strains. The expected band sizes for pSC5 are 20,808-, 15,855-, 7,314-, 6,848-, 3,189-, 1,610-, and 6-bp. Cbr—C. bromeliacearum, Ct—C. tolerans, Mb—M. borealis, Mp—M. pulcherrima, Mg—M. gruessi, Ml—M. lunata, Sc—S. cerevisiae, and Ca—C. auris. Ladder: 2-log ladder.

FIGS. 6A to 6C: Development of pSC5 plasmid compatible with GG assembly. 6A) Two versions of the pSC5 plasmid were created. Version 1 (pSC5GGv1) has the mRFP landing pad located in the middle of the plasmid, and version 2 (pSC5GGv2) has the mRFP located between yeast elements HIS3 and CEN6-ARS4. 6B) Example of GG assembly to insert a gene of interest (ShBle) into pSC5GGv1. pSC5—original plasmid, pSCGGv1-domesticated plasmid, pSC5GGv1_ShBle—a selected E. coli colony with ShBle inserted grown on LB plates supplemented with gentamicin (40 μg mL⁻¹). 6C) Conjugation of pSC5 and pSC5GGv1_ShBle to S. cerevisiae. NTC—nourseothricin, Zeo—zeocin.

FIGS. 7A to 7B: Conjugation-based antifungal. 7A) Diagram illustrating the predicted outcomes on S. cerevisiae colony formation following delivery of various plasmids via bacterial conjugation. The control strain (left) harboring pSC5 and pAGE2.0.T selected on synthetic yeast media lacking either histidine or tryptophan will survive. Conversely, the experimental strain (right) harboring pSC5-toxic gene plasmid and pAGE2.0.T when selected on synthetic yeast medium lacking histidine will die. 7B) Assay for S. cerevisiae colony formation on-HIS or -TRP media following bacterial conjugation with three toxic plasmids. pSC5-toxic1—partially toxic gene identified in A. laidlawii, pSC5-toxic2—H. influenzae HindII restriction gene inserted in pSC5GGv1, pSC5-toxic2—H. influenzae HindII restriction gene inserted in pSC5GGv2.

FIG. 8: Deletion plasmid assembly strategy. Each deletion plasmid was assembled from nine standard fragments as described in (Soltysiak et al. 2019) and two modified fragments amplified with original forward primer and new reverse primer and new forward primer and original reverse primer. After assembly, each deletion was genotyped by multiplex PCR.

FIG. 9: Bacterial conjugation from S. meliloti to S. cerevisiae. Representative plates of yeast transconjugants following conjugation from three S. meliloti clones harboring either pTA-Mob 2.0 or pSC5, plated on minimal media lacking histidine and containing ampicillin (100 μg mL⁻¹).

FIG. 10: Quantitative real-time polymerase chain reaction (qRT-PCR) of traJ expression. traJ mRNA expression in E. coli harboring the conjugative plasmids pTA-Mob 2.1 and pSC5.1 by qRT-PCR. The mean±SE are given for six biological replicates normalized to the reference genes rrsA and cysG. Student's T-test was used to carry out the pairwise comparison between pTA-Mob 2.1 and pSC5.1 and asterisks denote the significant difference between pairwise comparison (*, P<0.05; **, P<0.01).

FIG. 11: Bacterial conjugation from E. coli to diverse yeast species. Representative plates of yeast transconjugants following conjugation from E. coli harboring either pSC5 or pTA-Mob 2.0, plated on YPAD media supplemented with nourseothricin (100 μg mL 1) and ampicillin (100 μg mL⁻¹).

FIGS. 12A to 12C: Conjugation frequency of pSC5 to Metschnikowia gruessi. 12A) Three replicates of M. gruessi transconjugants plated on five YPDA plates supplemented with nourseothricin (100 μg mL⁻¹) and with ampicillin (100 μg mL⁻¹). 12B) Dilution series (10-3-10⁻⁵) of M. gruessi plated on YPDA plates supplemented with ampicillin (100 μg mL⁻¹). 12C) Average conjugation frequency of M. gruessi represents the mean±standard deviation for three biological replicates.

FIG. 13: Genotyping transconjugants of diverse yeast species. Genotyping of diverse yeast strains following conjugation with E. coli harboring pSC5. Multiplex PCR was performed to amplify the nourseothricin resistance gene of amplicon size 283-bp. Ct—Candida tolerans, Cbr—Candida bromeliacearum, Mb—Metschnikowia borealis, Mp—Metschnikowia pulcherrima, Ml—Metschnikowia lunata, Cbe—Candida aff. bentonensis, Cub—Candida ubatubensis, Mg—Metschnikowia gruessi, Sc—Saccharomyces cerevisiae, Ca—Candida auris, B—big colony, M—medium colony, and S—small colony. Clones highlighted in green were further analyzed with restriction enzyme digest and a phenotypic conjugation screen (FIG. 5, FIG. 14).

FIG. 14: Phenotypic E. coli to E. coli conjugation screen of recovered transconjugant plasmids from diverse yeast species. Selected recovered pSC5 plasmids from big diverse yeast colonies and S. cerevisiae colonies were transformed into E. coli and tested for conjugation to E. coli harboring pAGE1.0. Transconjugant E. coli were spot plated on LB plates supplemented with chloramphenicol (30 μg mL⁻¹) and gentamicin (60 μg mL⁻¹). Ct—Candida tolerans, Cbr—Candida bromeliacearum, Cbe—Candida bentonensis, Cub—Candida ubatubensis, Mb—Metschnikowi borealis, Mp—Metschnikowi pulcherrima, Ml—Metschnikowi lunata, Mg—Metschnikowi gruessi, Sc—S. cerevisiae, Ca—Candida auris, Ec—Escherichia coli, B—Big colony, -ve—Negative (S. cerevisiae only).

FIGS. 15A-15C. Testing of plasmid pSC6 and inducible conjugation from E. coli to E. coli. 15A: A schematic representing the movement of the inducible conjugative plasmid pSC6 which contains a gentamicin (GM) marker, to the recipient cell which contains a tetracycline (Tet) marker. It also shows arabinose binding to a regulatory protein and to enable expression of gene 11 (TrbF), enabling conjugation. 15B: During conjugation strains were either induced with arabinose (100 μg/mL) or remained uninduced. They were then plated on selection plates supplemented with gentamicin (40 μg/mL) and tetracycline (10 μg/mL). pSC5 induced and uninduced are the positive controls. 15C: Bar graph showing the average transconjugants/mL for each donor strain. A student's T-test was performed to analyze the difference between induced pSC6, which contains our inducible cassette, and induced pSC5, the positive control. There was a significant decrease in average transconjugants/mL in pSC6 (p=0.00018, n=3).

### DETAILED DESCRIPTION

### 1. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this disclosure belongs.

The articles “a” and “an” are used herein to refer to one or more than one (i.e., to at least one) of the grammatical object of the article.

As used herein, the term “comprising” is intended to mean that the compounds, compositions and methods include the recited elements, but not exclude others. “Consisting essentially of’ when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

Terms of degree such as “substantially”, “about” and “approximately” as used herein mean a reasonable amount of deviation of the modified term such that the end result is not significantly changed. These terms of degree should be construed as including a deviation of at least +/−5% of the modified term if this deviation would not negate the meaning of the word it modifies.

A “conjugative plasmid” is a plasmid that is transferred from one organism, such as a bacterial cell, to another organism during a process termed conjugation. The term refers to a self-transmissible plasmid that carries genes promoting the plasmid's own transfer by conjugation. Cis-conjugative plasmids carry their own origin of replication, oriV, and an origin of transfer, oriT, and genes promoting the plasmid's own transfer by the conjugation process. When conjugation is initiated, a relaxase enzyme creates a “nick” in one plasmid DNA strand at the oriT. The enzyme may work alone or in a complex of over a dozen proteins. The transferred, or T-strand, is unwound from the plasmid and transferred into the recipient bacterium in a 5′-terminus to 3′-terminus direction through a conjugative pilus. The remaining strand is replicated, either independent of conjugative action (vegetative replication, beginning at the oriV) or in concert with conjugative replication. Conjugation functions can be plasmid encoded, but some conjugation genes can be found in the bacterial chromosome or another plasmid and can exhibit their activity in trans to a separate plasmid that encodes the oriT sequence.

Numerous conjugative plasmids are known, which can transfer associated genes within one species (narrow host range) or between many species (broad host range). A Trans-conjugative plasmid is a plasmid that is mobilizable but not self-transmissible. Conjugation can occur between species classified as different at any taxonomic level—including in the extreme between domains, e.g., bacteria to eukaryotes.

“Food grade” bacteria are bacteria that are approved for human or animal consumption.oriT: Site of origin of transfer replication. The mechanism of plasmid transfer by bacterial conjugation includes a replication of the plasmid in which a break is introduced into one strand of duplex plasmid DNA, DNA replication then commences at the site of the break (oriT), together with transfer of the cut strand to the recipient cell.

Probiotic micro-organisms are known to have a beneficial effect on the health and well-being of the host. Typical probiotic bacteria that have been employed in this respect belong to the Lactobacillus or the Bifidobacterium genus, such as Bifidobacterium longum, Bifidobacterium lactis, Lactobacillus acidophilus, Lactobacillus rhamnosus, Lactobacillus paracasei, Lactobacillus johnsonii, Lactobacillus plantarum, Lactobacillus salivarius, Enterococcus faecium, Saccharomyces boulardii, Lactobacillus reuteri.

“Superior conjugative plasmid” in this document, means a conjugative plasmid that promotes an increased conjugation frequency from a bacterium to a recipient eukaryotic species relative the conjugative frequency observed with pTA-Mob 2.0 when tested in cis (mobilizing itself) and/or trans (mobilizing another plasmid).

### 2. Overview

The present disclosure relates to improved DNA transfer from bacteria to a recipient cell of a eukaryotic species by optimizing the traJ promoter.

A DNA fragment comprising cluster mutation in the relaxase operon, specifically in the traJ promoter, significantly improved DNA conjugative transfer from bacteria to recipient eukaryotic cells, such as S. cerevisiae and diverse yeasts, including the emerging pathogen C. auris. Improved, streamlined, and Golden Gate assembly-compatible plasmid derivatives of pTA-Mob 2.0 have been generated to enable facile insertion of custom genetic cassettes. The designer DNA fragments of the present disclosure can be used as a novel antifungal reagent, with important applications for the development of next-generation antifungal therapeutics.

As such, in one embodiment, the present disclosure provides for a DNA fragment for facilitating superior conjugative transfer of a transferable plasmid from a donor bacterium to a recipient eukaryotic cell. In embodiments, the DNA fragment comprises a mutated traJ promoter operatively linked to a traJ gene that enables improved transfer of the transferable plasmid by conjugation from the donor bacterium to the recipient eukaryotic cell relative to a native traJ promoter. In one embodiment, the mutated traJ promoter includes the nucleotide sequence ccgag (SEQ ID NO: 3). In another embodiment, the mutated traJ promoter comprises, consists essentially of, or consists of SEQ ID NO: 2.

In embodiments the transferable plasmid is a cis-conjugative plasmid (i.e., self-transmissible). In embodiments, the transferable plasmid is a trans-conjugative plasmid (not self-transmissible).

In one embodiment, the DNA fragment is included in the genome of the donor bacterium. In another embodiment, the DNA fragment is included in the transferable plasmid. In another embodiment, the DNA fragment is included in a non-transferable plasmid within the donor bacterium.

In embodiments, the DNA fragment includes a gene of interest. In embodiments, the gene of interest can be included in the genome of the donor bacterium or in a non-transferable plasmid within the donor bacterium.

In embodiments, the gene of interest is included in the transferable plasmid. In embodiments, when the transferable plasmid is transferred to the recipient eukaryotic cell, the gene of interest is expressed in the recipient cell to encode for a protein of interest.

In embodiments, the gene of interest is an inducible gene. The inducible gene can be located in the genome of the donor bacteria, in the transferable plasmid or on any non-transferable plasmid within the donor bacteria. In addition, the inducible gene can be transferred to a eukaryotic cell (for example to kill it) but it can also have a function in the donor bacterial cell (for example to build the conjugation machinery that allows for the transfer of DNA).

The DNA fragments carrying the mutant traJ promoter linked to the traJ gene of the present disclosure provides superior conjugative transfer of a transferable plasmid from a donor bacterial cell to a recipient cell of a eukaryotic species (i.e., eukaryotic cell). In embodiments the eukaryotic species is a species of the fungal kingdom.

In embodiments, novel DNA fragments have been developed and validated for improved conjugation efficiency between bacteria and diverse yeast species, including Saccharomyces cerevisiae, Candida auris, Candida bromeliacearum, Candida tolerans, Metschnikowia gruessii, Metschnikowia borealis, Metschnikowia pulcherrima and Metschnikowia lunata.

In embodiments, the recipient eukaryotic cell is an animal kingdom cell or a plant kingdom cell.

In embodiments, the recipient eukaryotic cell is a nematode cell.

In embodiment, the recipient eukaryotic cell is an algae cell.

In embodiments, the donor bacterium is a gram-negative bacterium, such as E. coli.

In embodiments, the donor bacterium is a gram-positive bacterium, including probiotics.

In embodiments, at least one of (i) the DNA fragment, (ii) the donor bacterium, (iii) the transferable plasmid, and (iv) any non-transferable plasmid within the donor bacterium, includes further mutations that in combination with the mutated traJ promoter improves the transfer of the transferable plasmid from the donor bacterium to the recipient eukaryotic cell.

In another embodiment, the present disclosure provides for bacteria having a transferable plasmid and a DNA fragment of the present disclosure. As previously described, the DNA fragment can be located in the genome of the bacteria, the transferable plasmid or in any non-transferable plasmid within the bacteria.

The bacteria of the present disclosure can be gram-negative such as E. coli, or gram-positive. In embodiments, the bacteria are probiotic bacteria. Non-limiting examples of probiotic bacteria that can be used as donor bacteria in the present disclosure include Lactobacillus acidophilus, Lactobacillus rhamnosus GG, Saccharomyces boulardii, Bifidobacterium bifidum and Bacillus coagulans.

In one embodiment, the present disclosure provides for a method of conjugatively transferring a gene of interest from a donor bacterium into a recipient eukaryotic cell, the method comprising: (a) providing the bacterium of the present disclosure in which the transferable plasmid includes the gene of interest, and (b) conjugatively transferring the transferable plasmid from the bacterium to the recipient eukaryotic cell, thereby transferring the gene of interest to the recipient eukaryotic cell.

In one embodiment of the method of transferring the gene of interest, the gene of interest is operably joined to a promoter functional in the recipient cell of the recipient eukaryotic cell.

In another embodiment, the gene of interest is not native to the donor bacterium.

The gene of interest includes genes that provide new functions to the recipient eukaryotic cell, delete genes from the recipient eukaryotic cell, and/or kills the recipient eukaryotic cell. For example, when the recipient eukaryotic cell is a cell of the fungal kingdom, the plasmid of the present disclosure may include a gene that expresses in the recipient cell an antifungal protein. In embodiments, the gene of interest is inducible.

### EXAMPLES

The examples are described for the purposes of illustration and are not intended to limit the scope of the disclosure. The Examples form part of the description.

### 1. Materials and Methods

### 1.1 Experimental Design

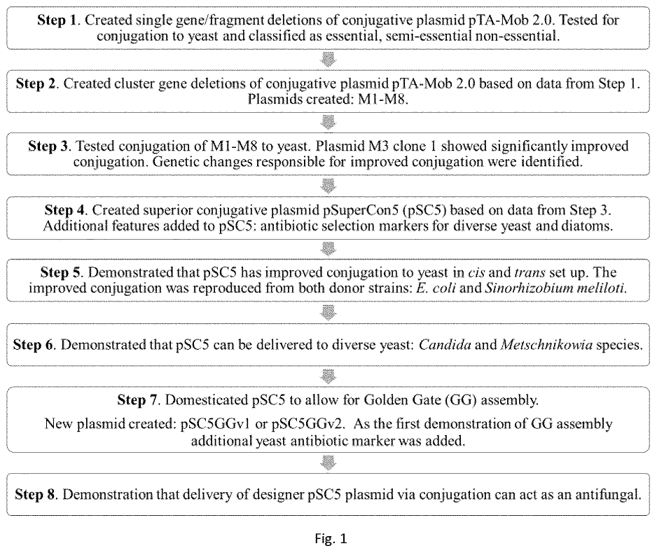

The eight-step flowchart shows experiments and major findings of the present disclosure is described in FIG. 1.

### 1.2 Microbial Strains and Growth Conditions

Saccharomyces cerevisiae VL6-48 (ATCC MYA-3666: MATα, his3-Δ200, trp1-Δ1, ura3-52, lys2, ade2-101, met14, psi+cir⁰) was grown at 30 °C in rich medium (2×YPDA: 20 g L⁻¹ yeast extract (BioShop Canada Inc., Cat #: YEX401.1, Canada), 40 g L⁻¹ peptone (BioShop Canada Inc., Cat #: PEP403.1, Canada), 40 g L⁻¹ glucose (BioShop Canada Inc., Cat #: GLU501.205, Canada), and 200 mg L⁻¹ adenine hemi-sulfate (MilliporeSigma, Cat #: A2545, Germany) supplemented with ampicillin (100 μg mL⁻¹; BioBasic, Cat #: AB0028, Canada)); or, grown with selection on either: 1) yeast synthetic complete medium lacking histidine supplemented with adenine hemi-sulfate (Teknova, Inc., Cat #: C7112, USA), 2) yeast synthetic complete medium lacking tryptophan (Teknova, Inc., Cat #: C7131, USA), 3) 2×YPDA supplemented with nourseothricin (100 μg mL⁻¹; Jena BioScience, Cat #: AB-102XL, Germany), or 4) 2×YPDA supplemented with zeocin (100 μg mL⁻¹; Invivogen, Cat #: ant-zn-5p, USA). Solid yeast media contained 2% agar (BioShop Canada Inc., Cat #AGA001.500, Canada). Following spheroplast transformation, all complete minimal media used contained 1 M sorbitol (BioShop Canada Inc., Cat #: SOR508.5, Canada). Escherichia coli Epi300 (Lucigen Corp., Cat #: LGN-EC300110, USA) was grown at 37 °C in Luria Broth (LB) supplemented with appropriate antibiotics (gentamicin (40 μg mL⁻¹; BioBasic, Cat #: GB0217, Canada) and chloramphenicol (15 μg mL⁻¹; BioBasic, Cat #: CB0118, Canada)). Solid media contained 1.5% agar. For transformation of E. coli SOC medium (20 g L⁻¹ tryptone, 5 g L⁻¹ yeast extract, 0.5 g L⁻¹ NaCl, 10 mL 250 mM KCl, 5 mL 2M MgCl₂, 20 mL 1M glucose) was used during the recovery time. Diverse yeasts, Metschnikowia gruessii (H53), Metschnikowia pulcherrima (CBS 5833), Metschnikowia lunata (BS 5946), Metschnikowia borealis (SUB 99-207.1), C. auris, Candida tolerans (UWOPS 98-117.5), Candida bromeliacearum (UNESP 00-103), Candida pseudointermedia (UWOPS 11-105.1), Candida ubatubensis (UNESP 01-247R), and Candida aff. bentonensis (UWOPS 00-168.1) were grown at 30 °C in 2×YPDA (All diverse yeast were obtained from Dr. Marc-Andre Lachance collection at Western University except C. auris came from https://wwwn.cdc.gov/arisolatebank/accession number SAMN05379609). Sinorhizobium meliloti (Rm4123 R-; obtained from Finan Lab, McMaster University) was grown at 30 °C in LBmc medium (10 g L⁻¹ tryptone, 5 g L⁻¹ yeast extract, 5 g L⁻¹ NaCl, 0.301 g L⁻¹ MgSO₄, and 0.277 g L⁻¹ anhydrous CaCl₂)) supplemented with appropriate antibiotics (gentamicin 40 μg mL⁻¹ and streptomycin 100 μg mL⁻¹; BioBasic, Cat #: SB0494, Canada). Solid media contained 1.5% agar.

### 1.3 Plasmid Construction

### 1.3.1 PCR Amplification

Plasmid fragments were amplified with GXL polymerase (Takara Bio Inc., Cat #: R050A, Japan) according to the manufacturer's instructions using annealing temperatures between 50-60 °C and 25-30 cycles.

### 1.3.2 Plasmid Assembly in Yeast

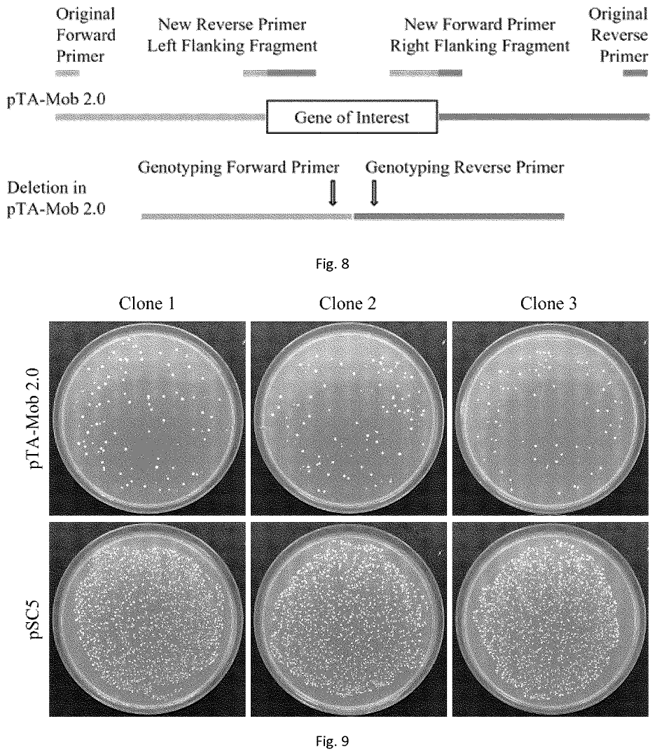

Plasmids were assembled in yeast as previously described²⁸. Primers for deletion plasmids are listed in Table 2 and all primers and templates used to generate other plasmids are listed in Table 3:1) Single gene/fragment deletion plasmids: pTA-Mob 2.0 plasmid was used as a template. Each plasmid was created with nine standard fragments as previously described²⁸ and two additional fragments designed as shown in FIG. 8. 2) Minimized plasmids (M1-8): Eight minimized conjugative plasmids (M1-8) were designed based on the results obtained for the pTA-Mob 2.0 deletion plasmids. pTA-Mob 2.0 or M3C1 plasmid was used as a template for PCR fragments listed in Table 3. 3) M3C1_F1-F5 hybrid plasmids: These plasmids were assembled by swapping the fragments between M3C1 and M3C2. 4) pTA-Mob 2.0 Tp and pTA-Mob 2.0 To: Primer-mediated mutagenesis was performed to introduce each mutation (Tp-in traJ promoter and To- in traJ ORF) into pTA-Mob 2.0. 5) Superior conjugative plasmid (pSC5): The pSC5 plasmid was derived from M3C1 plasmid by the addition of two versions of Nourseothricin N-acetyl transferase (NAT) genes which provide resistance for nourseothricin antibiotic. The first version was amplified from a plasmid pTA-Mob-NAT (unpublished, Karas lab) allowing selection in diatoms and referred to as dNAT; and the version was amplified from pGMO1 (unpublished, Karas lab), which contained an alternative genetic code for selection in diverse yeasts as previously described⁴³ and referred to as yNAT. The remaining fragments of the pSC5 plasmid were amplified from M3C1 as previously described²⁸. 6) pTA-Mob 2.1/pSC5.1. These two plasmids were created lacking the second copy of traJ located in the vector backbone. 7) Domesticated pSC5 (pSC5GGv1/v2): Two Bsal cut sites located within the fcpD Promoter and traC1 ORF were removed from pSC5 using primer-mediated mutagenesis. An RFP landing pad, consisting of a monomeric Red Fluorescent Protein (mRFP) gene driven by an arabinose-inducible pBAD promoter and a terminator was amplified from pAGE2.0-i (unpublished, Karas lab) using primers designed with new Bsal cut sites and homology either to directly downstream the native I-Scel restriction site (pSC5GGv1), or within the HIS3/CEN6/ARS4 element of the vector backbone (pSC5GGv2).

### 1.3.3 Golden Gate Assembly

1) Golden Gate (GG) assembly. For GG assembly, 20 fmol of plasmid and insert were mixed in a 15 μL reaction with 1.0 μL T4 DNA ligase (New England BioLabs, Inc., Cat #: M0202L, USA), 0.5 μl Bsal-HF V2 (New England BioLabs, Inc., Cat #: R3733S, USA), using the following conditions: 10 cycles of 37 °C for 5 minutes and 16 °C for 10 minutes followed by incubation at 37 °C for 5 minutes, 80 °C for 10 minutes and infinite hold at 12 °C Primers are listed in Table 3. 2) pSC5GGv1_ShBle. The zeocin (ShBle) resistance marker cassette was amplified with flanking Bsal cut sites from pRS32 (unpublished, Shapiro Lab) and GG assembly was performed with pSC5GGv1. The primers used are listed in Table 3. 3) pSC5-toxic1, pSC5-toxic2, and pSC5-toxic3. Three versions of toxic plasmids to kill yeast cells were created, one with an Acholeplasma laidlawii toxic gene (ACL0117) 44 and two versions with the restriction enzyme HindII. The A. laidlawii and HindII cassettes both contained an ACT1 yeast intron, flanking Bsal cut sites, and either an A. laidlawii or HindII toxic gene. The A. laidlawii toxic gene cassette was amplified in three fragments: the ACT1 yeast intron from S. cerevisiae VL6-48 gDNA, and the toxic gene in two halves from A. laidlawii PG-8A gDNA with primers listed in Table 3. The A. laidlawii toxic gene cassette was then constructed through a hierarchical GG assembly. First, the ACT1 yeast intron and the second half of the A. laidlawii toxic gene were assembled by GG assembly, and then 1 μL of the product was used as a template for PCR amplification of the joined fragments. Next, GG assembly was performed with 20 fmol of the PCR product with the first half of the toxic gene, and the complete toxic gene cassette was PCR amplified. The fully constructed cassette was then mixed with pSC5GGv1, and GG assembly was performed. The HindII cassette split by the ACT1 intron was flanked by URA3 promoter/terminator was synthesized (BioMatik, Canada), then PCR amplified and used in GG assembly with pSC5GGv1 or pSC5GGv2.

### 1.4 Plasmid Analysis

Screening in yeast. Following yeast assembly of the pTA-Mob 2.0 deletion plasmids, 20 individual yeast colonies were passed twice on solid media lacking histidine, and DNA was isolated and screened by multiplex PCR using the Qiagen Multiplex Kit (Qiagen, Inc., Cat #: 206143, Germany) according to the Qiagen Multiplex PCR Handbook. For all other plasmids following yeast assembly, colonies were pooled rather than individually screened. Transformation to E. coli. Total DNA was isolated as previously described²⁴. Isolated DNA (0.5-2 μL) was added to E. coli Epi300 electro-competent cells (40 μL) and electroporated using the Gene Pulser Xcell Electroporation System (2.5 kV voltage, 25 μF capacitance, 200 (resistance). Following a recovery in 1 mL of SOC medium for 1 hour at 37 °C (225 RPM), a 100-250 μL aliquot of the transformants were plated on LB medium supplemented with gentamicin (40 μg mL⁻¹). The cells transformed with Golden Gate-compatible plasmids (pSC5GGv1/v2, pSC5-toxic1, pSC5-toxic2, and pSC5-toxic3) were instead plated on LB plates containing gentamicin (40 μg mL⁻¹) and arabinose (100 g mL⁻¹). Screening in E. coli. For Golden Gate-compatible plasmids, white colonies were then screened by multiplex PCR for insertion of the cassette of interest. For all other assembled plasmids, the transformed E. coli was pooled and conjugated to S. cerevisiae, DNA was re-isolated and transformed back to E. coli to focus screening on functional conjugative plasmids. Once in E. coli, all plasmids were genotypically screened using multiplex PCR and restriction enzyme digest analysis. Sequencing. The plasmids, pTA-Mob 2.0 Tp/To, underwent Sanger DNA sequencing (London Regional Genomics Centre at Robarts Research Institute) to ensure the introduction of the correct mutations using the primers listed in Table 3. Selected plasmids were sequenced at CCIB DNA Core at Massachusetts General Hospital or at the Western University sequencing facility.

### 1.5 Conjugation

1.5.1 Both donor (E. coli) and recipient (S. cerevisiae) strains were prepared and frozen prior to conjugation experiments. For E. coli strains, saturated overnight cultures inoculated with a single colony were diluted to OD₆₀₀ of 0.1 in 50 ml of LB medium supplemented with appropriate antibiotics (Table 1) and grown until an OD₆₀₀ of 1.0 was reached. The cells were pelleted (3,000×RCF, 15 minutes) in a 50 mL Falcon tube and resuspended in 500 μL ice-cold 10% glycerol. Then, 100 μL aliquots in Eppendorf tubes were frozen in a −80 °C ethanol bath and stored at −80 °C For S. cerevisiae recipient strain preparation, a culture was started from a single colony and grown in 5 mL of 2×YPDA medium supplemented with ampicillin (100 μg mL⁻¹) for 7 hours. After, this culture was diluted in 50 mL of 2×YPDA medium supplemented with ampicillin (100 μg mL⁻¹) and grown until an OD₆₀₀ of 3.0 was reached (~17 hours). The cells were pelleted (3,000×RCF, 5 minutes) in a 50 mL Falcon tube and resuspended in 1 mL of ice-cold 10% glycerol. Then, 250 μL aliquots in Eppendorf tubes were frozen in a −80 °C ethanol bath, and stored at −80 °C

On the day of conjugation, conjugation plates (20 mL, 1.8% agar, 10% LB medium, complete minimal glucose broth lacking histidine) were dried for 30 minutes. Aliquots of the donor (E. coli) and recipient (S. cerevisiae) strains were removed from the freezer and thawed on ice for approximately 20 minutes. Next, 50 μL of S. cerevisiae was added to the 100 μL of E. coli and mixed by gentle pipetting before being transferred to the plate and spread evenly. Alternatively, when the yeast toxic plasmids were being tested, 10 μL of the recipient S. cerevisiae strain was used. Once dried, the plates were incubated at 30 °C for 3 hours, or 12 hours when wild yeast strains were used as the recipient. The plates were scraped with 2 mL of sterile double-distilled water (sddH₂O), mixed by vortexing for 5 seconds, and 100 μL plated on respective selection media (25 mL, 2% agar supplemented with ampicillin 100 μg mL⁻¹) listed in Table 1. In the case of wild yeast strains, they were plated on 1×YPDA medium supplemented with nourseothricin (100 μg mL⁻¹) and two technical replicates of each dilution (100-10⁻¹) were plated on selective plates. For experiments evaluating conjugation of pSC5 in cis and trans, dilution series of 10⁰-10⁻² were generated and plated on selective media while dilution series of Oct. 4, 2010-7 were generated and plated on non-selective medium (1×YPDA supplemented with ampicillin 100 μg mL⁻¹); and two technical replicates were plated for each dilution.

### 1.5.2 Bacterial Conjugation

### 1.5.2.1 E. coli to E. coli—Cis-Configuration

E. coli donor and recipient strains were prepared as in Method section 1.5.1 except the donor strains were resuspended in 5 mL of ice-cold 10% glycerol, and 500 μL aliquots were prepared in 1.5 mL Eppendorf tubes. To assess conjugation of pSC5 in bacteria, two donor strains of E. coli harboring either pSC5 or pTA-Mob 2.0 and an E. coli recipient strain harboring pAGE1.0 (chloramphenicol 15 μg mL⁻¹; Brumwell et al. 2019; Table 1) were prepared and stored in the −80 °C freezer. On the day of the conjugation, conjugation plates (20 mL, LB media with 1.5% agar) were prepared, and tubes containing the E. coli strains were removed from the freezer and thawed on ice. Once thawed, 10 μL of the donor E. coli strain was added to 100 μL of the recipient E. coli strain and mixed by pipetting prior to being transferred to the plate and spread evenly. Plates were incubated at 30 °C for 90 minutes and then were scraped with 1.5 mL of sterile double distilled water and mixed thoroughly by vortexing for 5 seconds. A dilution series (10⁻¹-10⁻⁸) was created in a 96-well plate and 100 UL of dilutions Oct. 1, 2010-4 were plated on selection plates (25 mL, LB media with 1.5% agar supplemented with chloramphenicol 15 μg mL⁻¹ and gentamicin 40 μg mL⁻¹). On non-selective plates (LB media with 1.5% agar supplemented with chloramphenicol 15 μg mL⁻¹) 100 μL of dilutions Oct. 1, 2010-8 were plated. Plates were incubated at 37 °C overnight and the following morning the colonies were counted, and conjugation frequency was calculated (transconjugant CFU/recipient CFU).

### 1.5.2.2 S. meliloti to S. cerevisiae—Cis-Orientation

S. meliloti was prepared similarly to E. coli; overnight cultures of a single colony were diluted to OD₆₀₀ of 0.1 in 50 mL of LB media with appropriate antibiotics (streptomycin 100 μg mL⁻¹ and gentamicin 40 μg mL⁻¹) and grown until an OD₆₀₀ of 1.0 was achieved. On the day of conjugation, the conjugation plates (20 mL, yeast synthetic complete medium lacking histidine 1.8% agar and 10% LBmc media) were made and the S. meliloti and S. cerevisiae cells were thawed on ice for approximately 20 minutes. Once thawed, 50 μL of S. cerevisiae was added to 100 μL of S. meliloti and mixed by gentle pipetting before being transferred to the plate and spread evenly. The plates were incubated at 30 °C for 3 hours. Next, the plates were scraped with 2 mL of sddH₂O and mixed thoroughly by vortexing for 5 seconds. For each conjugation, 3 biological replicates and 1 technical replicate were used and 100 μL of each dilution (10⁰-10⁻¹) for each sample was plated on selection plates (25 mL, yeast synthetic complete medium lacking histidine, 2% agar, supplemented with ampicillin (100 μg mL⁻¹)). The plates were incubated at 30 °C, were scored after 4 days, and conjugation frequency was calculated.

### 1.5.2.3. Conjugation from E. coli to S. cerevisiae—Cis—and Trans-Configuration

To assess bacterial conjugation of pSC5 to S. cerevisiae in cis-orientation two donor strains of E. coli harboring either pSC5 or pTA-Mob 2.0 (gentamycin 40 μg mL⁻¹) were prepared as in method section 2.5, and in trans-orientation two donor strains of E. coli harboring either pSC5 and pAGE2.0.T or pTA-Mob 2.0 and pAGE2.0.T (gentamycin 40 μg mL⁻¹ and chloramphenicol 15 μg mL⁻¹) were prepared as in method section 2.5 and all stored in the −80 °C freezer. On the day of conjugation, the conjugation plates (cis-20 mL, yeast synthetic complete medium lacking histidine 1.8% agar and 10% LB media; trans-20 mL, yeast synthetic complete medium lacking tryptophan 1.8% agar and 10% LB media) were prepared and the E. coli and S. cerevisiae cells were thawed on the ice for approximately 20 minutes. Once thawed, 50 μL of S. cerevisiae was added into the E. coli tube containing 100 μL of cells and mixed by pipetting before being transferred to the plate and spread evenly. The plates were incubated at 30 °C for 3 hours. Next, the plates were scraped with 2 mL of sddH₂O and mixed thoroughly by vortexing for 5 seconds. A dilution series (10⁻⁰-10⁻⁷) was generated and two technical replicates of 100 μL for dilutions 10⁰-10⁻⁴ were plated on selection plates (cis-25 mL, yeast synthetic complete medium lacking histidine 2% agar supplemented with ampicillin (100 μg mL⁻¹); trans-25 mL, yeast synthetic complete medium lacking tryptophan 2% agar supplemented with ampicillin (100 μg mL⁻¹)); and two technical replicates of 100 UL for dilutions for each sample (10⁻⁴-10⁻⁷) were plated on non-selective plates (25 mL, 1×YPDA supplemented with ampicillin (100 μg mL⁻¹)). The plates were incubated at 30 °C, were scored after 4 days, and conjugation frequency was calculated.

### 1.5.2.4. Conjugation from E. coli to Diverse Yeast and Transconjugant Analysis

Conjugation proceeded as described in Methods 2.5, except once dried, the conjugation plates were incubated at 30 °C for 12 hours and selection plates were incubated at 30 °C for 3 days before the number of colonies was counted.

To test the recovery of pSC5 plasmid from diverse yeast transconjugants, the plasmid was isolated from selected diverse yeast transconjugants. The recovered plasmids were transformed into E. coli by electroporation and re-conjugated back from E. coli to diverse yeast species following the protocol in Method 2.5, except on selective plates spot plating rather than full plates were used. After conjugation, cells were scraped with 2 mL of sddH₂O. These cells were serially diluted in 96-well plates and 5 μL of different dilutions (10⁰-10⁻⁴) were spot plated in 1×YPDA media supplemented with nourseothricin (100 μg mL⁻¹).

### 1.5.2.5. Conjugation-Based Kill Assay in S. cerevisiae

To assess yeast killing facilitated by bacterial conjugation, three donor E. coli strains harboring pAGE2.0.T and either pSC5-toxic1, pSC5-toxic2, or pSC5-toxic3 (gentamycin 40 μg mL⁻¹ and chloramphenicol 15 μg mL⁻¹) and the recipient S. cerevisiae were prepared as in Method section 2.5 and stored in the −80 °C freezer. On the day of conjugation, the conjugation plates (20 mL, yeast synthetic complete medium lacking histidine 1.8% agar and 10% LB media) were made and the E. coli and S. cerevisiae cells were thawed on ice for approximately 20 minutes. Once thawed, 10 μL of S. cerevisiae was added into the E. coli tube containing 100 μL of cells and mixed by pipetting before being transferred to the plate and spread evenly. The plates were incubated at 30 °C for 3 hours. Next, the plates were scraped with 2 mL of sddH₂O and mixed thoroughly by vortexing for 5 seconds. For each conjugation, 4 biological replicates and 1 technical replicate were used and 100 μL of each dilution (10⁰-10⁻¹) for each sample was plated on both selective plates (25 mL, yeast synthetic complete medium lacking histidine, and yeast synthetic complete medium lacking tryptophan, 2% agar, supplemented with ampicillin (100 μg mL⁻¹). The plates were incubated at 30 °C, colonies were scored after 4 days, and killing efficiency (CFU in-HIS media/CFU in-TRP media) was calculated.

### 1.6 RNA Isolation and Quantitative Reverse Transcriptase-Polymerase Chain Reaction

For RNA isolation, the E. coli strains carrying conjugative plasmids pTA-Mob 2.1 and pSC5.1 were grown in LB medium supplemented with gentamicin (40 μg mL⁻¹) for overnight at 37 °C In the morning, 1 mL of cells was diluted into 25 mL of LB medium supplemented with gentamicin (40 μg mL⁻¹) and grown for 120 minutes at 37 °C until optical density (OD₆₀₀) reached 1. Subsequently, the RNA stabilization of the culture was performed using RNAprotect Bacteria Reagent (Qiagen, Inc., Cat #: 76506, Germany). Briefly, 400 μL (2×10⁸ cells) of culture was transferred to a 15 mL Falcon tube containing 800 μL of RNAprotect Bacteria Reagent, and the suspension was vortexed for 5 seconds and incubated at room temperature for 5 minutes. Total RNA was isolated using the RNeasy Mini Kit (Qiagen, Inc., Cat #: 74104, Germany) according to the manufacturer's instructions. Following DNase treatment with TURBO DNA-free™ Kit (Invitrogen Cat #: AM1907, USA), the RNA concentration was determined using DeNovix (DeNovix Inc., USA) and the integrity was verified by running 400 ng of RNA on a 1% agarose (w/v) gel.

cDNA was prepared from 500 ng of RNA using the High-Capacity cDNA Reverse Transcription Kit (Applied Biosystems, Cat #: 4368814, USA). qRT-PCR was performed using six biological and three technical replicates, on a ViiA7 system of QuantStudio Real-Time PCR System (Applied Biosystems, USA) using the SYBR™ Select Master Mix (Applied Biosystems, Cat #: 472908, USA) under the following conditions: 50 °C for 2 minutes, 95 °C for 2 minutes followed by 40 cycles of 95 °C for 1 second, 60 °C for 30 seconds. Expression levels were normalized against two reference genes (rrsA and cysG) as previously described²³. Primer sequences used for the qRT-PCR expression analyses are listed in Table 3.

### 1.7 Statistical Analysis

The pairwise comparisons between groups were made using a Student's t-test with either equal or unequal variance based on the result of an F-test. Data were expressed as either ±95% confidence interval (CI) or as mean±standard error of the mean (SEM) of at least three biological replicates. The tests were considered statistically significant when P<0.05 (*), P<0.01 (**), or P<0.001 (***).

### 2. Results

### 2.1. Development of Streamlined Conjugation Plasmids

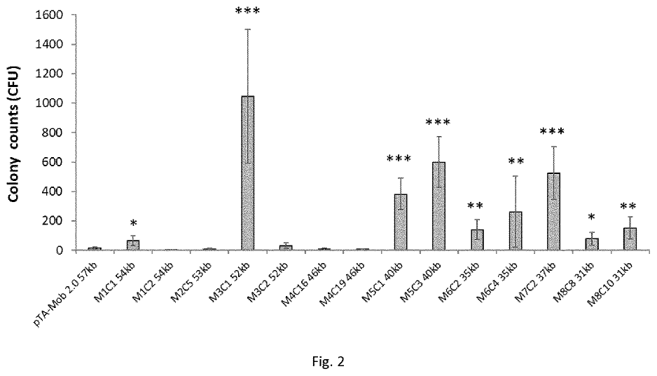

As a first step towards creating an optimized and minimized conjugative plasmid for yeast, 55 single genes or small genetic regions were individually deleted from our previously established trans-kingdom conjugation plasmid, pTA-Mob 2.028 (FIG. 8, Table 2). To validate these plasmid variants, up to two clones of each were tested for conjugation from E. coli to S. cerevisiae, and the genes/regions deleted were classified as essential (no conjugation), semi-essential (decreased conjugation), or non-essential (near wild type conjugation) for bacteria-to-yeast conjugation (Table 4). Based on this data, four streamlined plasmids were created where clusters of non-essential genes were simultaneously removed (plasmids M1-M4, Table 1, Table 3). E. coli containing plasmids M1-M4 were then conjugated to yeast, and we observed a significant increase in successful conjugation efficiency for plasmid M3 clone 1 (M3C1), monitored by yeast colony formation on selective media (FIG. 2). Sequencing both M3 clones, M3C1 and M3C2, revealed multiple mutations in each clone, which are likely responsible for the increase in conjugation efficiency for M3C1 (Table 5).

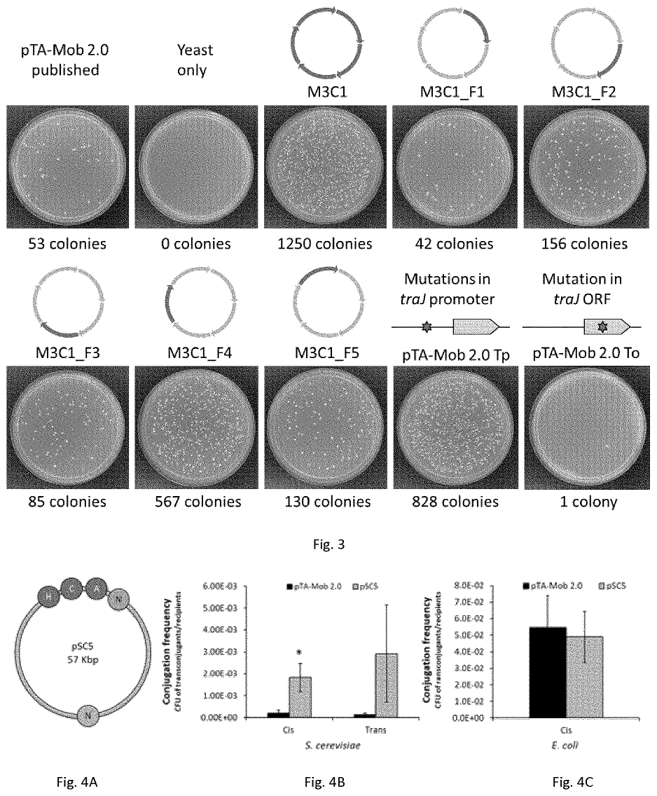

To identify which mutations in M3C1 were responsible for the increased conjugation efficiency, we performed a fragment swapping experiment between M3C1 and M3C2 to produce five hybrid plasmids M3C1_F1-F5 (FIG. 3). Each hybrid plasmid was created from four fragments that were amplified from M3C2 and one fragment from M3C1. Hybrid plasmid M3C1_F4 (fragment 4 originated from M3C1) had the closest conjugation efficiency compared to M3C1 (FIG. 3). There were two mutated regions in fragment 4 of M3C1: a cluster of mutations in the promoter of traJ, and a single mutation in the open reading frame (ORF) of traJ (Table 5). To validate which mutation(s) contributed to the increased conjugative phenotype, the promoter or ORF traJ mutations were introduced into pTA-Mob 2.0 and tested for conjugation efficiency. Only the mutations in the promoter region of traJ improved conjugation efficiency (FIG. 2). Additionally, we continued to minimize M3C1 by creating new plasmids with additional non-essential genes removed to obtain M5-M8 plasmids (FIG. 2). All M5-M8 minimized plasmids still produced more colonies when conjugated to yeast as compared to the original pTA-Mob 2.0 (FIG. 2).

### 2.2. Creation of Superior Conjugative Plasmid pSuperCon5

Based on the identified traJ promoter mutations, we created the pSuperCon5 (pSC5) plasmid with additional elements to enable the delivery of our improved conjugative plasmids to diverse yeast and diatoms. The pSC5 plasmid was built based on M3C1 and contains two copies of the nourseothricin resistance gene (yNAT and dNAT) added: one optimized for selection in diverse yeast and one for diatoms (FIG. 4A). Conjugation frequency for pSC5 from E. coli to S. cerevisiae was increased approximately 10- or 23-fold compared to pTA-Mob 2.0 when tested in cis (mobilizing itself) or trans (mobilizing another plasmid), respectively (FIG. 4B, Table 6 and Table 7). No significant difference in conjugation frequency was observed when plasmids were transferred between E. coli strains (FIG. 4C, Table 6 and Table 7).

In order to more precisely evaluate the frequency of bacteria-to-yeast conjugation, we performed additional experiments to monitor the effect of conjugation plasmid-containing E. coli on yeast viability. Cells from E. coli-to-yeast conjugation experiments were plated on non-selective yeast medium supplemented with ampicillin to inhibit E. coli growth. More yeast colonies grew when pSC5 was used versus pTA-Mob 2.0 (Table 8), indicating that E. coli carrying pSC5 has fewer adverse effects on yeast when they are co-cultured. To determine if the same effect could be observed when different donor cells were used, we performed conjugation with Sinorhizobium meliloti as a donor, as it was previously shown to conjugate to yeast 25. Similarly, a higher number of yeast colonies grew on non-selective plates when S. meliloti harboring pSC5 was used when compared to pTA-Mob 2.0 (Table 10). In addition, a higher number of colonies on selective plates were observed following conjugation of pSC5 from S. meliloti to S. cerevisiae compared to pTA-Mob 2.0 (FIG. 9, Table 10).

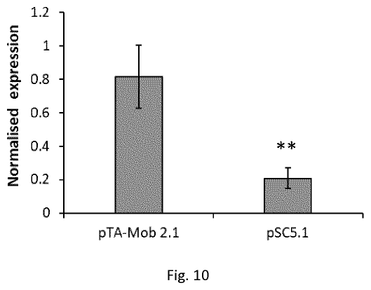

FIG. 10 shows the lower expression of traJ in E. coli harbouring plasmids carrying the promoter mutation.

### 2.3. Conjugation to Diverse Yeast Species

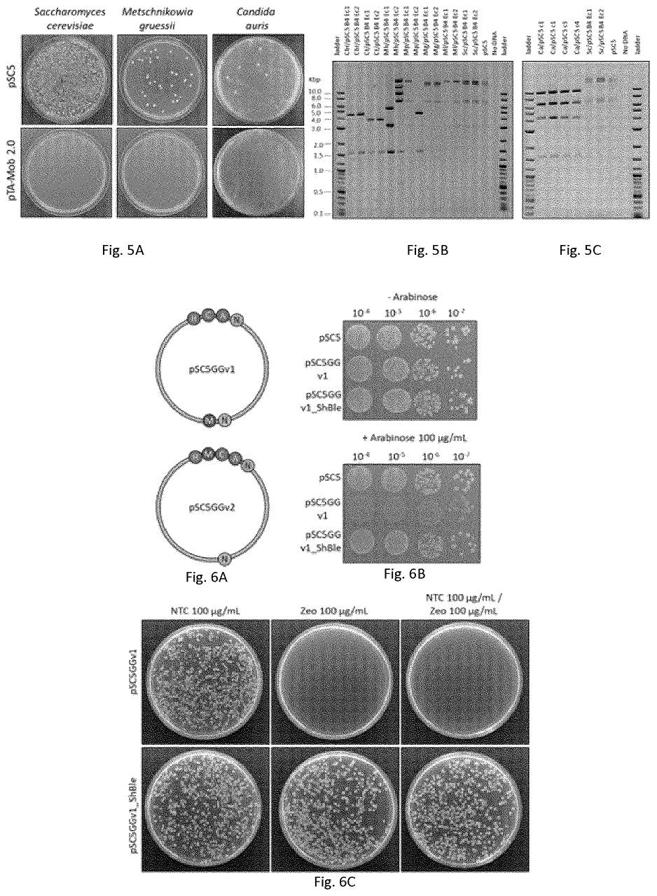

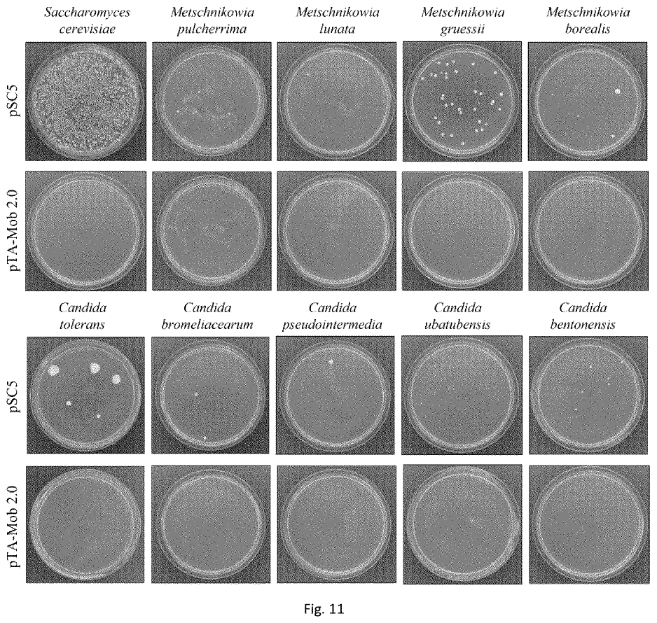

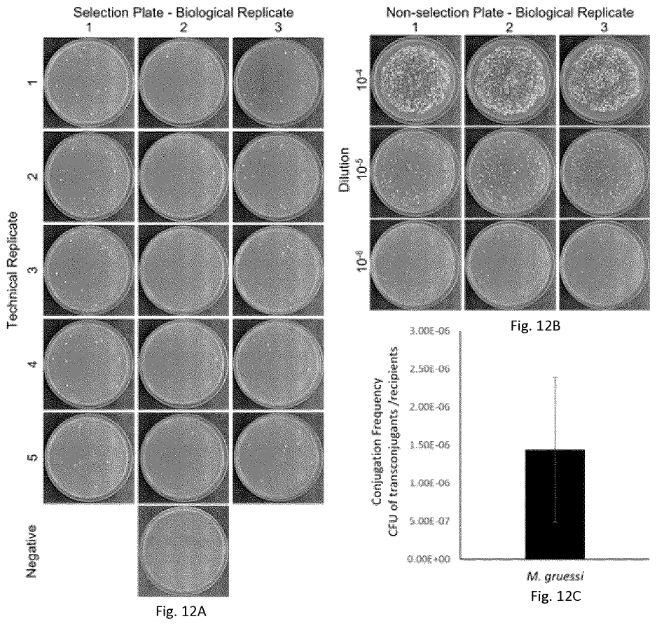

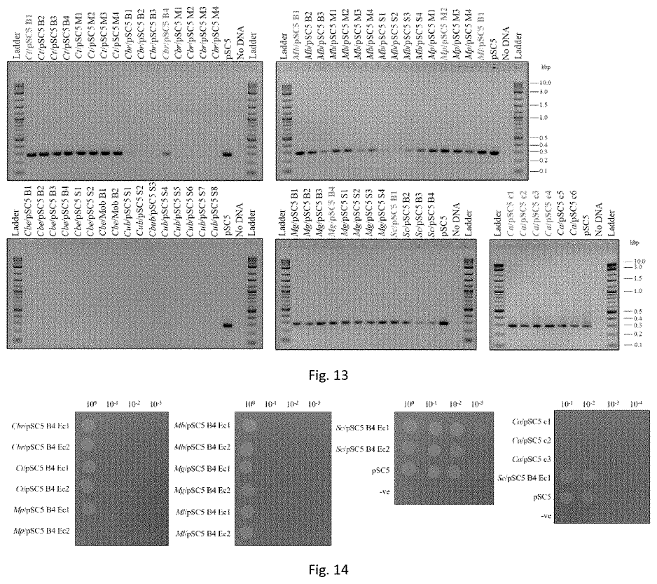

The significantly improved frequency of conjugation with the pSC5 plasmid suggests it may be effective for conjugation beyond a standard laboratory strain of S. cerevisiae and may have utility in transferring DNA from bacteria to diverse yeast species. To test the ability of pSC5 to transfer DNA to diverse yeast strains, we selected four Metschnikowia and six Candida species as conjugative recipients. Previously, we have demonstrated that small DNA fragments (yNAT selection marker) can be delivered to most of these species by electroporation⁴³. Conjugation to these diverse yeasts was performed with the same protocol used for S. cerevisiae, modified to allow for selection on complete yeast media containing antibiotics. Transconjugant colonies were obtained for all species (FIG. 5A, FIG. 11 and FIGS. 12A-12C), and 1-8 colonies for each species were genotyped by PCR for the presence of the yNAT marker. Of the 10 species tested, seven tested positive by PCR for the presence of the yNAT marker, suggesting successful conjugation had occurred (FIG. 13). A plasmid rescue experiment, where total yeast genomic DNA is electroporated into E. coli, was performed for selected colonies for each of the seven species, as well as S. cerevisiae. pSC5 plasmids from the seven yeast species were successfully recovered in E. coli, however, all except those recovered from S. cerevisiae showed rearrangements when diagnostic restriction enzyme digestion was performed (FIGS. 5B and 5C). Furthermore, only the plasmids recovered from S. cerevisiae were still able to conjugate (FIG. 14).

### 2.4. Domestication of pSC5 for Golden Gate Assembly

Next, we sought to modify the pSC5 plasmid to make it readily amenable to cloning for easy incorporation of any desired DNA fragment, to facilitate downstream applications of bacterial-to-yeast conjugation. To this end, we eliminated existing Bsal restriction sites from pSC5, and created a single Bsal-based Golden Gate cloning-compatible site to enable efficient plasmid manipulation^{45,46}. In addition, we incorporated a landing pad with mRFP driven by an arabinose inducible promoter (FIG. 6A). In this modified plasmid, Golden Gate assembly can readily be used to replace the mRFP gene with any gene of interest, allowing for an easy visual screen for correct gene insertion events (white versus red bacterial colonies).

To validate this system, we inserted a second antibiotic marker (ShBle) for yeast into pSC5GGv1 (FIG. 6A) to create pSC5GGv1_ShBle, which provides resistance to zeocin. White bacterial colonies were selected (FIG. 6B) and genotyped with diagnostic multiplex PCR and restriction digest (data not shown). These validated colonies were conjugated to S. cerevisiae and tested for survival on single or double antibiotic selection (FIG. 6C). Successful exconjugants which received pSC5GGv1_ShBle were able to grow on media supplemented with zeocin, nourseothricin or both (FIG. 6C).

### 2.5. Proof-of-Concept for Conjugation-Mediated Delivery as an Antifungal

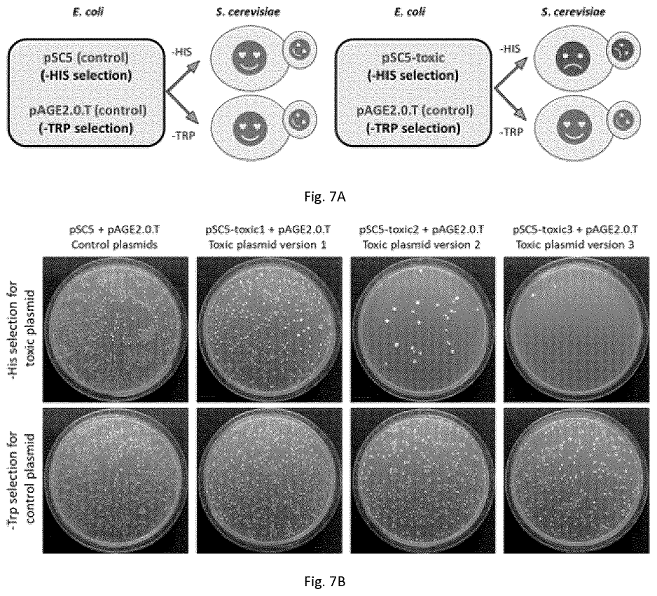

To demonstrate that pSC5-based conjugating plasmids could be used as an antifungal, we developed a system where each donor E. coli strain carried two plasmids: a control plasmid (pAGE2.0.T) that can be selected on media lacking tryptophan and either pSC5 or pSC5-toxic gene plasmid that can be selected on media lacking histidine (FIG. 7A). We used Golden Gate assembly to create three pSC5-toxic plasmids, each carrying a gene that should be partially or fully toxic to yeast. To prevent toxicity in E. coli, we inserted a yeast ACT1 intron⁴⁷ into each toxic gene. Next, we cloned the A. laidlawii toxic gene⁴⁴ into pSC5GGv1 to generate pSC5-toxic1, or an Haemophilus influenzae HindII restriction gene into pSC5GGv1 and pSC5GGv2 to generate pSC5-toxic2 and pSC5-toxic3, respectively. The pSC5 and pSC5-toxic gene plasmids can act in cis, mobilizing themselves, as well as in trans, mobilizing the control plasmid pAGE2.0.T. Using a control E. coli strain carrying plasmids pSC5 and pAGE2.0.T, we observed a similar colony number on both selection plates. For conjugation with pSC5-toxic gene plasmids, substantially fewer colonies grew on minimal media lacking histidine. The most substantial difference in yeast colony formation was with donor E. coli carrying pSC5-toxic3 (FIG. 7B, Table 11). This provides a proof-of-concept that bacteria to yeast conjugation can be used to effectively deliver plasmid-based antifungals.

### 2.6 Testing of the New Plasmid pSC6 and Inducible Conjugation from E. coli to E. coli

pSC6 Plasmid Construction

pSC6 plasmid is a derivative of superior conjugative plasmid (pSC5). For its construction, first, we removed two Bsal cut sites present in fcpD promoter and traC1 ORF of pSC5 plasmid using primer mediated mutagenesis. Out of two copies of traJ, the one which is located downstream to origin of transfer of vector backbone was removed. The remaining traJ in pSC6 includes the mutation in the traJ promoter of pSC5 that provides superior conjugative plasmids delivery operatively linked to the traJ gene. For this we amplified the fragment spanning traJ deletion using pSC5.1 as the template. The native trbF gene was also deleted from backbone. The fragment with trbF deletion was amplified using trbF knock out plasmid. Next, we amplified inducible trbF cassette with monomeric Red Fluorescent Protein (mRFP) gene fragment driven by an arabinose inducible pBAD promoter and terminator using trbF inducible plasmid as the template with primer mediated addition of Bsal restriction sites and homology to downstream of I-Scel restriction site in pSC5 backbone. All other fragments were amplified using pSC5GG-V1 as the template. Finally, these fragments were assembled in yeast to generate pSC6 plasmid.

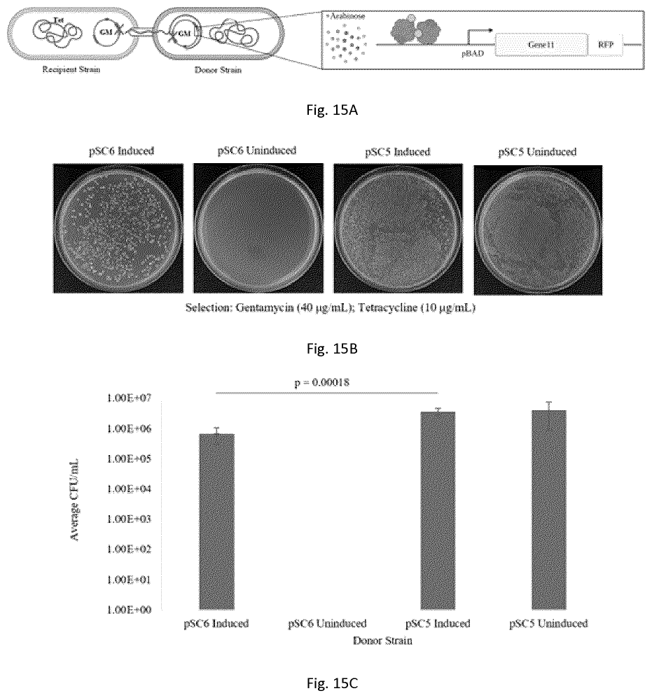

FIG. 15A shows a schematic representing the movement of the inducible conjugative plasmid pSC6 which contains a gentamicin (GM) marker, to the recipient cell which contains a tetracycline (Tet) marker. It also shows arabinose binding to a regulatory protein and to enable expression of gene 11 (TrbF), enabling conjugation.

During conjugation strains were either induced with arabinose (100 μg/mL) or remained uninduced. They were then plated on selection plates supplemented with gentamicin (40 μg/mL) and tetracycline (10 μg/mL). pSC5 induced and uninduced are the positive controls. The results are shown in FIG. 15B.

FIG. 15C is a bar graph showing the average transconjugants/mL for each donor strain. A student's T-test was performed to analyze the difference between induced pSC6, which contains our inducible cassette, and induced pSC5, the positive control. There was a significant decrease in average transconjugants/mL in pSC6 (p=0.00018, n=3).

It should be understood that since pSC6 includes the mutated traJ promoter operatively linked to the traJ gene, pSC6 also improves conjugative transfer from a donor bacterium to yeast relative to the native pTA-Mob 2.0 plasmid. It follows that the inducible cassette of this study can be used to induce conjugation from a donor bacterium to a recipient eukaryotic cell.

### 3. Discussion

Conjugation-based techniques, such as the one described here, provide a unique and functional method to deliver plasmids between microbial species in vitro and in vivo. While there are innumerable possible applications for these systems, many have focused on the use of plasmid-encoded CRISPR-based genetic manipulation systems to modify the genomes of the recipient microbes^{19,23,48}. Indeed, CRISPR-based gene targeting and manipulation systems offer a breadth of applications that can be paired with conjugation (or other methods of DNA delivery, such as phage transduction) to achieve desired manipulation of a target microbial population. The majority of this work to date has focused on bacterial species. For instance, CRISPR-based systems have been used to induce lethal DNA damage in key bacterial pathogens, including E. coli, Staphylococcus aureus, and Clostridium difficile, to effectively eradicate unwanted bacterial populations, including drug-resistant bacteria^{13,14,49}, and specific pathogenic species or sub-populations^{13,16-19,50,51}. In addition to directly killing bacterial populations, CRISPR systems can also be applied to modify virulence determinants to erode microbial pathogenicity^{50,52}, or alter drug-resistance genes to restore antimicrobial susceptibilities^{15,23,53-57} To enable the application of these CRISPR systems in vivo, many have relied on the use of bacterial conjugation or phage transduction as methods to deliver the relevant CRISPR components^{13,17,21-23,58}. While this has been effective for delivery to bacterial strains, it has limited the applications in fungi, which lack well-established tools for conjugation or virus-based gene delivery^{59,60}.

To address the bottleneck of improving DNA delivery to yeast, we performed experiments to optimize the conjugative plasmid pTA-Mob 2.028. We first evaluated whether plasmid derivatives with targeted deletions of the conjugative plasmid could improve DNA transfer to S. cerevisiae. After testing 57 single-gene and four cluster-gene deletion plasmids, one with superior conjugative properties (M3C1) was identified. Sequence analysis of M3C1 revealed that in addition to the designed deletions, M3C1 had unintended mutations that were likely introduced during PCR amplification or plasmid assembly. The mutations responsible for improved conjugation to S. cerevisiae were narrowed down to the promoter region of traJ (Tp) using a fragment swapping experiment. Following this discovery, five derivative plasmids of M3C1 were built containing the Tp mutation: four minimized versions (M5-M8) and pSuperCon5 (pSC5, containing selectable markers for diverse yeast species⁴³ and diatoms²⁴). Each derivative plasmid of M3C1, including the smallest 31 kb plasmid M8, outperformed the original 57 kb pTA-Mob 2.0 plasmid when tested for DNA transfer to S. cerevisiae. Notably, using pSC5 compared to pTA-Mob 2.0 we observed an increase in conjugation to S. cerevisiae 10- or 23-fold in either a cis or trans setup, respectively. Yet, no increase in plasmid transfer was observed when pSC5 was conjugated between E. coli strains. This improved conjugation to S. cerevisiae could be partially explained by the increased S. cerevisiae viability during the co-culture conjugation step when plasmids harboring the Tp mutation are used. The same effect was also observed when S. meliloti was used as a conjugative donor, suggesting the mechanism may be independent of the bacterial host. We also demonstrated that the Tp mutation results in a lower expression of the traJ gene. TraJ has been demonstrated as an essential conjugative protein that negatively autoregulates the expression of the relaxase operon⁶¹

Therefore, decreased expression of traJ could have a significant effect on the expression of all the conjugative machinery proteins. Further investigation will focus on resolving the link between traJ downregulation and increased yeast viability or DNA transfer during the co-culture conjugation step.

The significantly improved pSC5 plasmid allowed for DNA transfer to seven Metschnikowia and Candida yeast species, though relatively few colonies were obtained for each of them. One explanation for the low conjugative transfer could be that the S. cerevisiae centromere along with the origin of replication was not functional in these yeasts. In such a case, survival of these yeasts would only be possible if the conjugative plasmid was integrated into the yeast genome. Using a plasmid rescue experiment we showed that plasmids could be recovered in E. coli, although none of them had the correct size or ability to conjugate. Since E. coli can assemble linear fragments into plasmids⁶², it is most likely that some of the linear yeast fragments with integrated conjugative plasmids were assembled into plasmids in E. coli. Despite not being able to replicate as an episome in diverse yeasts, the improved conjugative plasmids, especially pSC5GGv1_ShBle with two antibiotic resistance genes, provide a great initial resource for DNA delivery. For applications where replicative plasmids are necessary for the yeast species of interest, specific origins and centromeres will need to be identified and incorporated into the conjugative plasmid as was done for S. cerevisiae^{63,64}.

Our improved conjugative plasmids hold promise as a novel antifungal. As a proof of concept, we cloned restriction nucleases onto pSC5GGv2 plasmid and demonstrated that >99% of yeast cells that receive the plasmid DNA can be eliminated. However additional improvements in the conjugation frequency will need to be achieved before this technology can be used in antifungal treatments. In the future, our Golden Gate-compatible plasmids can be engineered with programmable systems such as CRISPR/Cas9 to target specific yeast strains. This coincides with the development and optimization of numerous CRISPR-based editing platforms optimized for a diversity of yeast species⁶⁵⁻⁶⁹, including Candida pathogens⁷⁰. Recent work has demonstrated the utility of CRISPR systems for modifying fungal genes involved in virulence⁷¹⁻⁷⁸ and antifungal drug resistance^{71,74,79,80} in diverse Candida pathogens, and combining these CRISPR systems with this trans-kingdom conjugation system could facilitate the delivery of CRISPR to fungi in different environmental contexts.

TABLE 1
Plasmids used in this study.

| | Plasmid | E. coli | Yeast | |
|---|---|---|---|---|
| Plasmid | Size (kb) | Marker | Marker | Citation |
| pTA-Mob 2.0 | 57 | aacC1 | HIS3/URA3 | Soltysiak et al., |
| | | | | 2019 |
| pAGE1.0 | 18 | cat | HIS3 | Brumwell et al., |
| | | | | 2019 |
| pAGE2.0.T | 19 | cat | TRP1 | This Study |
| pAGE2.0-i | 20 | cat | HIS3 | This Study |
| pAGE2.0-iTraJ | 20 | cat | HIS3 | This Study |
| pRS32 | 11 | bla | ShBle | Shapiro et al. |
| | | | | (unpublished) |
| M1C1: pTA-Mob 2.0 ΔTrbO-FiwA | 54 | aacC1 | HIS3/URA3 | This Study |
| M1C2: pTA-Mob 2.0 ΔTrbO-FiwA | 54 | aacC1 | HIS3/URA3 | This Study |
| M2C5: pTA-Mob 2.0 ΔKlaC-KleA | 53 | aacC1 | HIS3/URA3 | This Study |
| M3C1: pTA-Mob 2.0 ΔIstA-TraB | 52 | aacC1 | HIS3 | This Study |
| M3C2: pTA-Mob 2.0 ΔIstA-TraB | 52 | aacC1 | HIS3 | This Study |
| M4C16: pTA-Mob 2.0 ΔIstA-TraE | 46 | aacC1 | HIS3 | This Study |
| M4C19: pTA-Mob 2.0 ΔIstA-TraE | 46 | aacC1 | HIS3 | This Study |
| M5C1: pTA-Mob 2.0 ΔTrbN-TraE | 40 | aacC1 | HIS3 | This Study |
| M5C3: pTA-Mob 2.0 ΔTrbN-TraE | 40 | aacC1 | HIS3 | This Study |
| M6C2: pTA-Mob 2.0 ΔTrbN-TraE, | 35 | aacC1 | HIS3 | This Study |
| ΔKlaC-KleA | | | | |
| M6C4: pTA-Mob 2.0 ΔTrbN-TraE, | 35 | aacC1 | HIS3 | This Study |
| ΔKlaC-KleA | | | | |
| M7C2: pTA-Mob 2.0 ΔTrbN-TraE, | 37 | aacC1 | HIS3 | This Study |
| ΔTrfA-TraJ^{†} | | | | |
| M8C8: pTA-Mob 2.0 ΔTrbM-TraE, | 31 | aacC1 | HIS3 | This Study |
| ΔKlaC-KleA, ΔTrfA-TraJ^{†} | | | | |
| M8C10: pTA-Mob 2.0 ΔTrbM-TraE, | 31 | aacC1 | HIS3 | This Study |
| ΔKlaC-KleA, ΔTrfA-TraJ^{†} | | | | |
| M3C1_F1: pTA-Mob 2.0 ΔIstA-TraB | 52 | aacC1 | HIS3 | This Study |
| M3C1_F2: pTA-Mob 2.0 ΔIstA-TraB | 52 | aacC1 | HIS3 | This Study |
| M3C1_F3: pTA-Mob 2.0 ΔIstA-TraB | 52 | aacC1 | HIS3 | This Study |
| M3C1_F4: pTA-Mob 2.0 ΔIstA-TraB | 52 | aacC1 | HIS3 | This Study |
| M3C1_F5: pTA-Mob 2.0 ΔIstA-TraB | 52 | aacC1 | HIS3 | This Study |
| pTA-Mob 2.0 Tp | 56 | aacC1 | HIS3/URA3 | This Study |
| pTA-Mob 2.0 To | 56 | aacC1 | HIS3/URA3 | This Study |
| pSC5: pTA-Mob 2.0 ΔIstA-TraB | 56 | aacC1 | HIS3/yNAT | This Study |
| pTA-Mob 2.1: pTA-Mob 2.0 ΔTraJ^{†} | 56 | aacC1 | HIS3/URA3 | This Study |
| pSC5.1: pTA-Mob 2.0 ΔIstA-TraB, | 55 | aacC1 | HIS3/yNAT | This Study |
| ΔTraJ^{†} | | | | |
| pSC5GGv1: pTA-Mob 2.0 | 57 | aacC1 | HIS3/yNAT | This Study |
| ΔIstA-TraB, +mRFP | | | | |
| pSC5GGv2: pTA-Mob 2.0 | 57 | aacC1 | HIS3/yNAT | This Study |
| ΔIstA-TraB, +mRFP | | | | |
| pSC5GGv1_ShBle: pTA-Mob 2.0 | 57 | aacC1 | HIS3/yNAT/ | This Study |
| ΔIstA-TraB | | | ShBle | |
| pSC5-toxic1: pTA-Mob 2.0 ΔIstA- | 59 | aacC1 | HIS3/yNAT | This Study |
| TraB, +ACL0117 | | | | |
| pSC5-toxic2: pTA-Mob 2.0 ΔIstA- | 57 | aacC1 | HIS3/yNAT | This Study |
| TraB, +HindII | | | | |
| pSC5-toxic3: pTA-Mob 2.0 ΔIstA- | 57 | aacC1 | HIS3/yNAT | This Study |
| TraB, +HindII | | | | |

aacC1 provides resistance to gentamicin; cat to chloramphenicol; bla to ampicillin; yNAT to nourseothricin in diverse yeast; ShBle to zeocin resistance gene in diverse yeasts. HIS3 is required for the histidine biosynthesis; URA3 for uracil biosynthesis; TRP1 for tryptophan biosynthesis in S. cerevisiae.
^{†}duplicated copy that is present in the plasmid backbone.

TABLE 2
Description of pTA-Mob 2.0 deletion plasmid library. The fragment split by
PCR amplification is listed, and regions of pTA-Mob 2.0 deleted are reported with respect
to the 3′ end of backbone insertion re-indexed to position 1. Diagnostic multiplex primers
used to screen deletion plasmids and their respective amplicon sizes are shown. Genes
either completely or partially removed from pTA-Mob 2.0 in each deletion plasmid are
listed, their location within pTA-Mob 2.0 is annotated in parentheses, and the primers
used to remove them are provided.

| | Frag- | | | | | Gene(s) Deleted |
|---|---|---|---|---|---|---|
| Deletion | ment | Region | SEQ ID | | Amplicon | Or Partially |
| Plasmid | Split | Deleted | NO | MPX Primers | (bp) | Deleted |
| 1 | 2 | 574-1241 | 4 5 | F - aggcggtaaaggtgagcag | 301 | upf16.5 (574- |
| | | | | R - gaagcctgcgaagagttgc | | 1241) |
| 2 | 2 | 1216-2364 | 6 7 | F - ctctgtttatcggcagttcg | 327 | trfA1 (1216- |
| | | | | R - gtattcgtgcagggcaagat | | 2364) & trfA2 |
| | | | | | | (1216-2073) |
| 3 | 2 | 1216-2073 | 8 9 | F - ctctgtttatcggcagttcg | 306 | trfA2 (1216- |
| | | | | R - tcgatggtccagcaagctac | | 2073) & trfA1 |
| | | | | | | (1216-2364) |
| 4 | 2 | 2938-3249 | 10 11 | F - ctcggtgtcacgggtaagat | 393 | trbA (2884- |
| | | | | R - cacgacgtaggggttctgat | | 3249) |
| 5 | 2 | 3583-4479 | 12 13 | F - tgcgctttgacagttgtttt | 374 | trbB (3520- |
| | | | | R - ttcgtcagccagctctcata | | 4479) |
| 6 | 2 | 4492-4929 | 14 15 | F - attcaccgaaacccattgag | 349 | trbC (4492- |
| | | | | R - catgattcggaacgcataga | | 4929) |
| 7 | 2 | 4932-5239 | 16 17 | F - ttcttccgaaccctgatctt | 304 | trbD (4932- |
| | | | | R - tccttggaacgatgcttttt | | 5243) |
| 8 | 2/3 | 5244-7794 | 18 19 | F - gtcggtctgatcctgtggtt | 338 | trbE (5240- |
| | | | | R - gtctgcttttgcgacacaac | | 7798) |
| 9 | 3 | 7799-8553 | 20 21 | F - cgtctctacgacctggcact | 352 | trbF (7795- |
| | | | | R - gtacccgcctcccacttct | | 8553) |
| 10 | 3 | 8565-9458 | 22 23 | F - ggtcagcaccgaaatcactt | 319 | trbG (8565- |
| | | | | R - agctgccgtacttggaggt | | 9458) |
| 11 | 3 | 9462-9944 | 24 25 | F - acggcaagaaaaccatcatc | 331 | trbH (9462- |
| | | | | R - tgaggtacatcggcatgttg | | 9944) |
| 12 | 3 | 9949-11340 | 26 27 | F - agcaacctgtatcgcctgac | 313 | trbI (9949- |
| | | | | R - tcctcgatttgcttctggac | | 11340) |
| 13 | 3/4 | 11357-12133 | 28 29 | F - gcagatgatcgccaaaaact | 312 | trbJ (11357- |
| | | | | R - gggcacttcttcaacagctc | | 12133) |
| 14 | 4 | 12145-12354 | 30 31 | F - caaatccgtggccttctg | 349 | trbK (12145- |
| | | | | R - acggtcaaggtccagaacag | | 12354) |
| 15 | 4 | 12361-13947 | 32 33 | F - gatctgccgaaggtcacg | 329 | trbL (12361- |
| | | | | R - cttgtggatgccgaagtacc | | 13947) |
| 16 | 4 | 13971-14570 | 34 35 | F - gttcgacgacaacagccttt | 324 | trbM (13971- |
| | | | | R - aagaatgttggccggaatct | | 14570) |
| 17 | 4 | 14586-15290 | 36 37 | F - gccatgcctataccgacttc | 336 | trbN (14586- |
| | | | | R - tgcccagtaccagaagatca | | 15290) |
| 18 | 4 | 15321-15584 | 38 39 | F - ggcagctcatcatcaacaac | 300 | trbO (15321- |
| | | | | R - ccaggacgaaaacgaaaaga | | 15584) |
| 19 | 4 | 15621-16355 | 40 41 | F - aagcgccgtttgatcttct | 321 | trbP (15621- |
| | | | | R - catggggatgttcagcagt | | 16355) |
| 20 | 4 | 16372-16791 | 42 43 | F - acgtactacccgctgcatct | 343 | upf31.7 (16372- |
| | | | | R - gatagaacgcttcggtgtcc | | 16791) |
| 21 | 4/5 | 16806-17501 | 44 45 | F - ataaccagctcgccatcaag | 318 | fiwA (16806- |
| | | | | R - cagcaccaggaacatcgtc | | 17501) |
| 22 | 5 | 17537-18199 | 46 47 | F - cgagctgctgaacaaggttt | 303 | upf32.8 (17537- |
| | | | | R - gagccaggtcaaacgagtgt | | 18199) |
| 23 | 5 | 18223-18882 | 48 49 | F - gactacaccgagggggaaag | 331 | parA1 (18223- |
| | | | | R - gagaaggacaccgaccgtta | | 18882) & parA2 |
| | | | | | | (18223-18846) |
| 24 | 5 | 18223-18846 | 50 51 | F - gactacaccgagggggaaag | 326 | parA2 (18223- |
| | | | | R - gtgggtcaacatggagctg | | 18846) & parA1 |
| | | | | | | (18223-18882) |
| 25 | 5 | 18843-19688 | 52 53 | F - tctcctggcgttcaagattc | 217 | parB (18843- |
| | | | | R - tccgtcatgtcgattgtcag | | 19376) & parC |
| | | | | | | (19373-19666) & |
| | | | | | | parA1 |
| | | | | | | (18223-18882) & |
| | | | | | | parA2 |
| | | | | | | (18223-18846) |
| 26 | 5 | 19373-19666 | 54 55 | F - tgcttgtctaccagcacgtc | 318 | parC (19373- |
| | | | | R - tccgtcatgtcgattgtcag | | 19666) |
| 27 | 5 | 19817-20068 | 56 57 | F - catggcgcattacagcaata | 304 | parD (19817- |
| | | | | R - gggaaagagttcgctcatgt | | 20068) & parE |
| | | | | | | (20065-20376) |
| 28 | 6 | 23490-24286 | 58 59 | F - cgaggttggtggtaatcgtt | 328 | istB (23489- |
| | | | | R - gccgcatagtgtagccagat | | 24286) |
| 29 | 6 | 24763-24993 | 60 61 | F - agactagccaccaccattgc | 328 | aphA (24763- |
| | | | | R - acggaggtagcagcagaaaa | | 24993) |
| 30 | 6 | 25077-25367 | 62 63 | F - tgcaatttcatctcctgctg | 308 | traA (25077- |
| | | | | R - cgcaacgtctaccagttcag | | 25367) |
| 31 | 6 | 25377-25815 | 64 65 | F - gttgacgatgatgctgttgg | 347 | traB (25375- |
| | | | | R - gccaggtgagaagtgctgtc | | 25815) |
| 32 | 6/7 | 25831-29016 | 66 67 | F - accttcatggtcagccattc | 314 | traC1 (25831- |
| | | | | R - gagttcgacccggaagaag | | 29016) & traC2 |
| | | | | | | (25831-28071) |
| 33 | 6 | 25831-28071 | 68 69 | F - accttcatggtcagccattc | 308 | traC2 (25831- |
| | | | | R - atcaaggcgctacaagagga | | 28071) & traC1 |
| | | | | | | (25831-29016) |
| 34 | 7 | 29023-29286 | 70 71 | F - tgtaacgcttcccggtagtc | 340 | traD (29023- |
| | | | | R - agtctccgagctgcacaagt | | 29286) |
| 35 | 7 | 29292-31505 | 72 73 | F - accaggtcaatgtcgctctc | 342 | traE (29292- |
| | | | | R - gccgaggttctgcttatgtc | | 31505) |
| 36 | 7 | 31520-32049 | 74 75 | F - tagtcctccgggtctagcaa | 345 | traF (31520- |
| | | | | R - gcgacatggttgtgtacgtc | | 32053) |
| 37 | 7 | 32054-33953 | 76 77 | F - gcttttggtggtgttgacct | 316 | traG (32050- |
| | | | | R - tgattatgttggtgcgctgt | | 33957) |
| 38 | 7/8 | 33958-36131 | 78 79 | F - tttttcgcccgtatctgtg | 505 | traH (34253- |
| | | | | R - gtatccaacggcgtcagaat | | 34612) & traI |
| | | | | | | (33954-36152) |
| 39 | 7 | 34253-34612 | 80 81 | F - gcaccagaatctcgtcgttt | 316 | traH (34253- |
| | | | | R - gtcaacggcacagcagagt | | 34612) & traI |
| | | | | | | (33954-36152) |
| 40 | 8 | 36149-36190 | 82 83 | F - ctcggtcttgccttgctc | 488 | traX (36149- |
| | | | | R - gtatccaacggcgtcagaat | | 36190) traI |
| | | | | | | (33954-36152) & |
| | | | | | | traJ (36187- |
| | | | | | | 36558) |
| 41 | 8 | 36187-36558 | 84 85 | F - gcgaagtcgctcttcttgat | 333 | traJ (36187- |
| | | | | R - gctctttggcatcgtctctc | | 36558) & traX |
| | | | | | | (36149-36190) |
| 42 | 8 | 36796-37199 | 86 87 | F - tcgctataatgaccccgaag | 338 | traK (36796- |
| | | | | R - gttgcgcgagttaatttcgt | | 37200) |
| 43 | 8 | 37201-37921 | 88 89 | F - aacggggaaggtcaagttct | 335 | traL (37200- |
| | | | | R - tgatggtatgcaggatcagc | | 37925) |
| 44 | 8 | 37926-38359 | 90 91 | F - ggcaagagctttgagcagat | 312 | traM (37922- |
| | | | | R - tgtcaaacaagcccagctaa | | 38359) |
| 45 | 8 | 38411-39758 | 92 93 | F - ggcgaaggtaatgaaggaca | 348 | upf54.4 (38412- |
| | | | | R - tgcgaaaagcatcacctatg | | 39758) |
| 46 | 8/9 | 40292-41218 | 94 95 | F - tggttctcggtctggacaat | 305 | kfrA (40292- |
| | | | | R - acctgccgttaagtcgagaa | | 41218) |
| 47 | 9 | 41932-42444 | 96 97 | F - ggatagcttgcaacatcagga | 295 | korF (41941- |
| | | | | R - acccggacaagctgaaaaag | | 42444) |
| 48 | 9 | 44843-45792 | 98 99 | F - ttggttgaacatagcggtga | 313 | klaC (44843- |
| | | | | R - gttcagacgacgctcatcaa | | 45796) |
| 49 | 9/10 | 45793-46875 | 100 | F - ctggtcgctgaatgtcgat | 392 | klaB (45793- |
| | | | 101 | R - gctcatcaccaccaagaaca | | 46929) & klaC |
| | | | | | | (44843-45796) |
| 50 | 10 | 46947-47720 | 102 | F - catgtcgaaggcgacgatag | 317 | klaA (46947- |
| | | | 103 | R - ttcgatggatgtttgctttg | | 47720) |
| 51 | 10 | 47843-48157 | 104 | F - gacagctcgttgagggaatc | 332 | kleF1 (47843- |
| | | | 105 | R - ggtactgaccgcactcacct | | 48157) & kleF2 |
| | | | | | | (47843-48013) |
| 52 | 10 | 47839-48013 | 106 | F - caaaacctcccccttcaatc | 318 | kleF2 (47843- |
| | | | 107 | R - tttgagcaatgccaagacag | | 48013) & kleF1 |
| | | | | | | (47843-48157) |
| 53 | 10 | 48185-48508 | 108 | F - ctccggattgaaccagtacg | 341 | kleE (48185- |
| | | | 109 | R - aagtcaccaagtgggtcgag | | 48508) |
| 54 | 10 | 48620-48838 | 110 | F - cacgcctgggaacttgataa | 319 | kleD (48620- |
| | | | 111 | R - gaatgctattgccgagaagc | | 48838) |
| 55 | 10 | 48854-49084 | 112 | F - ctcgacccacttggtgactt | 324 | kleC (48854- |
| | | | 113 | R - caggggaaaggtgttttcaa | | 49084) |
| 56 | 10 | 49237-49452 | 114 | F - gcttctcggcaatagcattc | 338 | kleB (49237- |
| | | | 115 | R - actggtccacccaggaagtc | | 49452) |
| 57 | 10 | 49501-49734 | 116 | F - ggctaagggtcgaaatgga | 336 | kleA (49501- |
| | | | 117 | R - actttacgccaagggagagg | | 49734) |

| | | Additional primers for splitting the fragment of interest |
|---|---|---|
| | | Top primer pairs with the original reverse primer for the |
| | | fragment of interest. |
| Deletion | SEQ ID | Bottom primer pairs with the original forward primer of the |
| Plasmid | NO | fragment of interest. |
| 1 | 118 | gcggcagagatgaacacgaccatcagcggctgcacagcgccattgacccaggcgtgttcc |
| | 119 | tgcgaggcagcggcctggtggaacacgcctgggtcaatggcgctgtgcagccgctgatg |
| 2 | 120 | tggctgctgaacccccagccggaactgaccccacaaggcctcaccctccttgcgggattg |
| | 121 | tgccccggcgtgagtcggggcaatcccgcaaggagggtgaggccttgtggggtcagttcc |
| 3 | 122 | tggctgctgaacccccagccggaactgaccccacaaggccagttcctcgcgtgtcgatgg |
| | 123 | aggtttggcgaagtcgatgaccatcgacacgcgaggaactggccttgtggggtcagttcc |
| 4 | 124 | atttttcaccaacatccttcgtctgctcgatgagcggggccgccaagggttagggcttgc |
| | 125 | tagcggctaaagaaggaagtgcaagccctaacccttggcggccccgctcatcgagcagac |
| 5 | 126 | gaccatcaaggagcgggccaagcgcaagctggaacgcgacggagtatttccaatgacaac |
| | 127 | tcagacggaacggaacagccgttgtcattggaaatactccgtcgcgttccagcttgcgct |
| 6 | 128 | cggccagtacatcaccaaaaccctgtaaggagtatttccatcatggctctgcgcacgatc |
| | 129 | ttgcctgcgcgacggatggggatcgtgcgcagagccatgatggaaatactccttacaggg |
| 7 | 130 | atgccgtgcgtgcggtagcggctggacggctcgcctaatcatgatccaagcaattgcgat |
| | 131 | cgccgaggcccgcgattgcaatcgcaattgcttggatcatgattaggcgagccgtccagc |
| 8 | 132 | ccgcgagaacaccaatagccaagggaagcaataccgatgaatgagttttgcagacacgat |
| | 133 | tcttgaagatcaagcccttgatcgtgtctgcaaaactcattcatcggtattgcttccctt |
| 9 | 134 | ccggggcctcgcccttgatgaatacctggaggcagcatgaggcactgaattatgaaaaag |
| | 135 | aggaccaaagcaaacagttcctttttcataattcagtgcctcatgctgcctccaggtatt |
| 10 | 136 | gggacttctcctggtcgagacttctgtgaggcactgaattaccatgcgtaagattctgac |
| | 137 | tggccgcgagtgcgatgacggtcagaatcttacgcatggtaattcagtgcctcacagaag |
| 11 | 138 | cagccaggaccgcgtgaccatttcaagggggaactaaaccgccaatgagcgaagatcaaa |
| | 139 | tggcgatgcgtccggtgccatttgatcttcgctcattggcggtttagttcccccttgaaa |
| 12 | 140 | atggtcccggccggcgcatgggttcggaaggagtaagccactccaaggagtaacttatga |
| | 141 | aacattcttagcgagcttcttcataagttactccttggagtggcttactccttccgaacc |
| 13 | 142 | ccctaccaggcgtttgactattaactccaaggagtaacttggggaggcgcgatgaagaaa |
| | 143 | gcaactgcgatgaagttggatttcttcatcgcgcctccccaagttactccttggagttaa |
| 14 | 144 | accgcgcaagcccgtctaagacctggtgaggggaggcgcgtgacgtatgaaaatccagac |
| | 145 | ccgcgagcgcggcagctctagtctggattttcatacgtcacgcgcctcccctcaccaggt |
| 15 | 146 | aggcttcaagcccagcgaaaagaaagagtggtgatgacgtaacgactcttaggagctacg |
| | 147 | gcttttttcagttgcatggtcgtagctcctaagagtcgttacgtcatcaccactctttct |
| 16 | 148 | agcccgcccaatcctgaaacgactcttaggagctacgaccggggaggggatagcgatgcc |
| | 149 | gtgccagcagcttggcaaacggcatcgctatcccctccccggtcgtagctcctaagagtc |
| 17 | 150 | cgcctaccgtcggttcgagcggtaaggggaggggatagcgggaggaacggccgtttagcg |
| | 151 | gaatgcccataggctttagccgctaaacggccgttcctcccgctatcccctccccttacc |
| 18 | 152 | cacgccataaggaggaacggccgtttagcggctaaagcctcgccctgcagggcgttctta |
| | 153 | tcatgccctcccccttggagtaagaacgccctgcagggcgaggctttagccgctaaacgg |
| 19 | 154 | atagcgccctgcagggcgttcttactccaagggggagggccctttgagattccaatatgc |
| | 155 | ggtgcatttcttgagcaattgcatattggaatctcaaagggccctcccccttggagtaag |
| 20 | 156 | gcgggctacttgtttttcacctgacctttgagattccaatggggagggcggcggatgctg |
| | 157 | aggaagcccttcaaccgtgtcagcatccgccgccctccccattggaatctcaaaggtcag |
| 21 | 158 | cgcttgacgatgcgaagttctactgaggggagggcggcggctacaaccgtgcgcaaggcg |
| | 159 | tacatacatcctccctaatgcgccttgcgcacggttgtagccgccgccctcccctcagta |
| 22 | 160 | tctgactacaaccgtgcgcaaggcgcattagggaggatgtgaaaagccgggcactgcccg |
| | 161 | cgcagcagcaaaaataaagccgggcagtgcccggcttttcacatcctccctaatgcgcct |
| 23 | 162 | gggacgcgaaaaggtgagaaaagccgggcactgcccggctttccacggctcgacggcgtg |
| | 163 | tcggcctctggtccgatccgcacgccgtcgagccgtggaaagccgggcagtgcccggctt |
| 24 | 164 | gggacgcgaaaaggtgagaaaagccgggcactgcccggctccttcgtccctccggttgtt |
| | 165 | ggaaatggcgacgcgagagcaacaaccggagggacgaaggagccgggcagtgcccggctt |
| 25 | 166 | tgctcgtccgtactggcgcgcaggtagatgcgggcgacctggcgcattacagcaatacgc |
| | 167 | gcgctaggcgcatttaaattgcgtattgctgtaatgcgccaggtegcccgcatctacctg |
| 26 | 168 | agcgcggcagcggcgcgcagcatggcgtagcttcggcgctttgggcctagtctagccggc |
| | 169 | gtattgctgtaatgcgccatgccggctagactaggcccaaagcgccgaagctacgccatg |
| 27 | 170 | tgtgctaatgtggttacgtgtattttatggaggttatccacggcctacatcctcacggct |
| | 171 | cgtagatcggcttcggcctcagccgtgaggatgtaggccgtggataacctccataaaata |
| 28 | 172 | ggaacatcgaccactgagtgcctatgaggagctgttgtgacgaaagtatcttagcgggca |
| | 173 | accgttatttgccattttcatgcccgctaagatactttcgtcacaacagctcctcatagg |
| 29 | 174 | gaacgatattgatcgagaagagccctgcgcagccgctgccggcgatgccccctegacctc |
| | 175 | tcctgaacgcctccctgatcgaggtcgagggggcatcgccggcagcggctgcgcagggct |
| 30 | 176 | ctcacaaagaaagccgggcaatgcccggctttttctgctgacgcctcctagatcgagcgc |
| | 177 | aaggccgagcagaaacgctcgcgctcgatctaggaggcgtcagcagaaaaagccgggcat |
| 31 | 178 | gaaaatcgtgcgggtacgcctcgatgttcatacgcctcctacgctctagttctccttagt |
| | 179 | gcaacgccgcgcgagaacctactaaggagaactagagcgtaggaggcgtatgaacatcga |
| 32 | 180 | gtgccccgatctgtactttgttcatacgctctagttctccagccaattacctcccgtcat |
| | 181 | cacgacgaccgcggccgccaatgacgggaggtaattggctggagaactagagcgtatgaa |
| 33 | 182 | gtgccccgatctgtactttgttcatacgctctagttctccagttgccccctgcgcaggct |
| | 183 | aacgacccagcaggccatcgagcctgcgcagggggcaactggagaactagagcgtatgaa |
| 34 | 184 | cctgctcgtggaacggctttttgacctctgccatagccaacgctttcactcctggttggt |
| | 185 | acagcaaaggccgtaacggcaccaaccaggagtgaaagcgttggctatggcagaggtcaa |
| 35 | 186 | aagtcgtcctgctgcacggtcttgggatcattcategcttatatccccctaccctcacca |
| | 187 | tcatcaggccggttctgacctggtgagggtagggggatataagcgatgaatgatcccaag |
| 36 | 188 | gcgattacaaggcgttcaaattgcatatatccccctaccctcatatcgtgatcccctccc |
| | 189 | gacggccaccgtcgaggaaggggaggggatcacgatatgagggtagggggatatatgcaa |
| 37 | 190 | cgatggcgacgtacttggtgaggcgctggaagcggctcattcatctactcctacctcggg |
| | 191 | gcgaggctcccttaaaactacccgaggtaggagtagatgaatgagccgcttccagcgcct |
| 38 | 192 | cccgtatctgtggccccacggcgttgtttcggttcttcatggggacgtgcttggcaatca |
| | 193 | aacggccggggggtgcgcgtgattgccaagcacgtccccatgaagaaccgaaacaacgc |
| 39 | 194 | tcggcaacatatttctcggccgccgcgatctgttcgggctgtgcttgctccttcgtcagt |
| | 195 | gagcgccgccgtcaagaactactgacgaaggagcaagcacagcccgaacagatcgcggcg |
| 40 | 196 | tcgctcttcttgatggagcgcatggggacgtgcttggcaaggctctgccctcgggcggac |
| | 197 | gcaaggtcatgatgggcgtggtccgcccgagggcagagccttgccaagcacgtccccatg |
| 41 | 198 | aatcacgcgcaccccccggccgttttagcggctaaaaaagccgcttgccgaattctgacg |
| | 199 | ttccttggtgtatccaacggcgtcagaattcggcaagcggcttttttagccgctaaaacg |
| 42 | 200 | atgcaggaaattactgaactgaggggacaggcgagagacgatggcgaaaattcacatggt |
| | 201 | ccccgcccttgccctgcaaaaccatgtgaattttcgccatcgtctctcgcctgtcccctc |
| 43 | 202 | caccttcaacccaacaccggacaaaaaggatctactgtaaatgagcgaccagattgaaga |
| | 203 | ccgcaatctcccggatcagctcttcaatctggtcgctcatttacagtagatcctttttgt |
| 44 | 204 | cggcctgtttgaacagctcgacgcggcggccgtgctatgaatcgcagaggcgcagatgaa |
| | 205 | gcccggcaacgccgggctttttcatctgcgcctctgcgattcatagcacggccgccgcgt |
| 45 | 206 | gcagatgaaaaagcccggcgttgccgggcttgtttttgcggacgcctccctttttagccg |
| | 207 | cgcactcgttagagttttagcggctaaaaagggaggcgtccgcaaaaacaagcccggcaa |
| 46 | 208 | ttgttccatctattttagtgaactgcgttcgatttatcaggttggctccggtaattggta |
| | 209 | ggtaagagtattattattcttaccaattaccggagccaacctgataaatcgaacgcagtt |
| 47 | 210 | cttgcaacatcaggagccgtttcttttgttcgtcagtcattcttcggtcctccttgtagc |
| | 211 | gccgcacagacaacggttccgctacaaggaggaccgaagaatgactgacgaacaaaagaa |
| 48 | 212 | aaaacaaaagcccggaaaccgggctttcgtctcttgccgctcatacggactcctgttggg |
| | 213 | ccagcccgtgcgcgagctggcccaacaggagtccgtatgagcggcaagagacgaaagccc |
| 49 | 214 | aggccgcctacctgggcgaaaacatcggtgtttgtggcatgtcgaaggcgacgatagggg |
| | 215 | aacgacgcacgacgccaaggcccctatcgtcgccttcgacatgccacaaacaccgatgtt |
| 50 | 216 | tgcgtcgttttcagtgcgttcatagggttctcccgccgtgtcgatacaccctcgcggtgg |
| | 217 | tccatcgaaaagcaattaacccaccgcgagggtgtatcgacacggcgggagaaccctatg |
| 51 | 218 | ccgctaaaatttggggacaggtcatttacagaaagccagctgctcaaagctccttgaagg |
| | 219 | ggagggtcaagcaagcggccccttcaaggagctttgagcagctggctttctgtaaatgac |
| 52 | 220 | ttagccgctaaaatttggggacaggtcatttacagaaagcgccccagccctcggcctccg |
| | 221 | atcggcgtactggttcaatccggaggccgagggctggggcgctttctgtaaatgacctgt |
| 53 | 222 | cctgtcttggcattgctcaaagctccttgaaggggccgcttgcttgaccctccacggcga |
| | 223 | ctggacgaattgaacacgcatcgccgtggagggtcaagcaagcggccccttcaaggagct |
| 54 | 224 | atcgaggtcaagcgccccggagaaatccggggcgtcatcccatcatcccctggcgtcagt |
| | 225 | ggcgaaattccgggccggtcactgacgccaggggatgatgggatgacgccccggatttct |
| 55 | 226 | agttcaccctatctcctacttgcatcatcatcccctggcgcggattggcctccggtaatt |
| | 227 | gccgggtagattcccaggtcaattaccggaggccaatccgcgccaggggatgatgatgca |
| 56 | 228 | gaggcgtcatgcttgaaaacacctttcccctggcgtgcaaggccctatctccttgagaga |
| | 229 | ggcccggctacggtcgggcctctctcaaggagatagggccttgcacgccaggggaaaggt |
| 57 | 230 | ctccttgagagaggcccgaccgtagccgggcctcgttccgcggggttgatcctccggttg |
| | 231 | gggcacttcgcccaggtcagcaaccggaggatcaaccccgcggaacgaggcccggctacg |

TABLE 3
List of primers used to amplify the assembly fragments and genotype the plasmids
created in this study.

| | | | | | |
|---|---|---|---|---|---|
| | SEQ | | | Expected | |
| | ID | | | size | |
| Fragment | NO | Primer | Primer Sequence (5′ to 3′) | (bp) | Template |

M1

| | | | | | |
|---|---|---|---|---|---|
| F1 | 232 | F | tgccgccgcgcgcatggtcgtaatgggaccgatagc | 5782 | pTA-Mob 2.0 |
| | | | ccgt | | |
| | 233 | R | tttaacctacttcctttggttccgggggatctcgcgactc | | |
| F2 | 234 | F | atcgaagagaagcaggacga | 6373 | pTA-Mob 2.0 |
| | 235 | R | tgctggtccatgaagatgaa | | |
| F3 | 236 | F | tcgagctgatgtttgacgac | 6137 | pTA-Mob 2.0 |
| | 237 | R | ggacttgaggttgctctgct | | |
| F4 | 238 | F | gtggacattggtttcagcaa | 3693 | pTA-Mob 2.0 |
| | 239 | R | gcgagaacctactaaggagaactagagcgtacgtgc | | |
| | | | ttcgaaccactcggagggacggtt | | |
| F5 | 240 | F | aaccgtccctccgagtggttcgaagcacgtacgctct | 3476 | pTA-Mob 2.0 |
| | | | agttctccttagtaggttctcgc | | |
| | 241 | R | aagcgatgaatgatcccaag | | |
| F6 | 242 | F | gagcaatggatagccgatgt | 6206 | pTA-Mob 2.0 |
| | 243 | R | aagcgatgaatgatcccaag | | |
| F7 | 244 | F | tgtaacgcttcccggtagtc | 6295 | pTA-Mob 2.0 |
| | 245 | R | cattgcaaagcgactgatgt | | |
| F8 | 246 | F | gatccgctccttgaactctg | 6259 | pTA-Mob 2.0 |
| | 247 | R | aggcccttgccaatgaat | | |
| F9 | 248 | F | ttctttgaatgcgcgggcgtcctggtgagcgtagtcca | 6000 | pTA-Mob 2.0 |
| | | | gc | | |
| | 249 | R | cgttcccgcctgcccctgattggcccgctgatcgacc | | |
| | | | gct | | |
| F10 | 250 | F | aatgttgcaaggcgatcag | 5745 | pTA-Mob 2.0 |
| | 251 | R | agccctcccgtatcgtagtt | | |

M2

| | | | | | |
|---|---|---|---|---|---|
| F1 | 252 | F | tgccgccgcgcgcatggtcgtaatgggaccgatagc | 5782 | pTA-Mob 2.0 |
| | | | ccgt | | |
| | 253 | R | tttaacctacttcctttggttccgggggatctcgcgactc | | |
| F2 | 254 | F | atcgaagagaagcaggacga | 6373 | pTA-Mob 2.0 |
| | 255 | R | tgctggtccatgaagatgaa | | |
| F3 | 256 | F | tcgagctgatgtttgacgac | 6137 | pTA-Mob 2.0 |
| | 257 | R | ggacttgaggttgctctgct | | |
| F4 | 258 | F | ggaccaggcgcagtccaccatcaacggcctgatgag | 6000 | pTA-Mob 2.0 |
| | | | cgcc | | |
| | 259 | R | atcggcgtgaagcccaacagggcca | | |
| F5 | 260 | F | gtggacattggtttcagcaa | 6273 | pTA-Mob 2.0 |
| | 261 | R | agctcatgcatcacaacagc | | |
| F6 | 262 | F | gagcaatggatagccgatgt | 6206 | pTA-Mob 2.0 |
| | 263 | R | aagcgatgaatgatcccaag | | |
| F7 | 264 | F | tgtaacgcttcccggtagtc | 6295 | pTA-Mob 2.0 |
| | 265 | R | cattgcaaagcgactgatgt | | |
| F8 | 266 | F | gatccgctccttgaactctg | 6259 | pTA-Mob 2.0 |
| | 267 | R | aggcccttgccaatgaat | | |
| F9 | 268 | F | ttctttgaatgcgcgggcgtcctgg | 4224 | pTA-Mob 2.0 |
| | 269 | R | tgaacacgcatcgccgtggagggtcaagcagcggc | | |
| | | | aagagacgaaagcccggtttccggg | | |
| F10 | 270 | F | cccggaaaccgggctttcgtctcttgccgctgcttgac | 2664 | pTA-Mob 2.0 |
| | | | cctccacggcgatgcgtgttca | | |
| | 271 | R | agccctcccgtatcgtagtt | | |

M3

| | | | | | |
|---|---|---|---|---|---|
| F1 | 272 | F | tgccgccgcgcgcatggtcgtaatgggaccgatagc | 5782 | pTA-Mob 2.0 |
| | | | ccgt | | |
| | 273 | R | tttaacctacttcctttggttccgggggatctcgcgactc | | |
| F2 | 274 | F | atcgaagagaagcaggacga | 6373 | pTA-Mob 2.0 |
| | 275 | R | tgctggtccatgaagatgaa | | |
| F3 | 276 | F | tcgagctgatgtttgacgac | 6137 | pTA-Mob 2.0 |
| | 277 | R | ggacttgaggttgctctgct | | |
| F4 | 278 | F | ggaccaggcgcagtccaccatcaacggcctgatgag | 6000 | pTA-Mob 2.0 |
| | | | cgcc | | |
| | 279 | R | atcggcgtgaagcccaacagggcca | | |
| F5 | 280 | F | gtggacattggtttcagcaa | 3693 | pTA-Mob 2.0 |
| | 281 | R | gcgagaacctactaaggagaactagagcgtacgtgc | | |
| | | | ttcgaaccactcggagggacggtt | | |
| F6 | 282 | F | aaccgtccctccgagtggttcgaagcacgtacgctct | 3476 | pTA-Mob 2.0 |
| | | | agttctccttagtaggttctcgc | | |
| | 283 | R | aagcgatgaatgatcccaag | | |
| F7 | 284 | F | tgtaacgcttcccggtagtc | 6295 | pTA-Mob 2.0 |
| | 285 | R | cattgcaaagcgactgatgt | | |
| F8 | 286 | F | gatccgctccttgaactctg | 6259 | pTA-Mob 2.0 |
| | 287 | R | aggcccttgccaatgaat | | |
| F9 | 288 | F | ttctttgaatgcgcgggcgtcctggtgagcgtagtcca | 6000 | pTA-Mob 2.0 |
| | | | gc | | |
| | 289 | R | cgttcccgcctgcccctgattggcccgctgatcgacc | | |
| | | | gct | | |
| F10 | 290 | F | aatgttgcaaggcgatcag | 5745 | pTA-Mob 2.0 |
| | 291 | R | agccctcccgtatcgtagtt | | |

M4

| | | | | | |
|---|---|---|---|---|---|
| F1 | 292 | F | tgccgccgcgcgcatggtcgtaatgggaccgatagc | 5782 | pTA-Mob 2.0 |
| | | | ccgt | | |
| | 293 | R | tttaacctacttcctttggttccgggggatctcgcgactc | | |
| F2 | 294 | F | atcgaagagaagcaggacga | 6373 | pTA-Mob 2.0 |
| | 295 | R | tgctggtccatgaagatgaa | | |
| F3 | 296 | F | tcgagctgatgtttgacgac | 6137 | pTA-Mob 2.0 |
| | 297 | R | ggacttgaggttgctctgct | | |
| F4 | 298 | F | ggaccaggcgcagtccaccatcaacggcctgatgag | 6000 | pTA-Mob 2.0 |
| | | | cgcc | | |
| | 299 | R | atcggcgtgaagcccaacagggcca | | |
| F5 | 300 | F | gtggacattggtttcagcaa | 3476 | pTA-Mob 2.0 |
| | 301 | R | ggttctgacctggtgagggtagggggatatacgtgctt | | |
| | | | cgaaccactcggagggacggtt | | |
| F6 | 302 | F | aaccgtccctccgagtggttcgaagcacgtatatccc | 3655 | pTA-Mob 2.0 |
| | | | cctaccctcaccaggtcagaacc | | |
| | 303 | R | tgtaacgcttcccggtagtc | | |
| F7 | 304 | F | gatccgctccttgaactctg | 6259 | pTA-Mob 2.0 |
| | 305 | R | aggcccttgccaatgaat | | |
| F8 | 306 | F | ttctttgaatgcgcgggcgtcctggtgagcgtagtcca | 6000 | pTA-Mob 2.0 |
| | | | gc | | |
| | 307 | R | cgttcccgcctgcccctgattggcccgctgatcgacc | | |
| | | | gct | | |
| F9 | 308 | F | aatgttgcaaggcgatcag | 5745 | pTA-Mob 2.0 |
| | 309 | R | agccctcccgtatcgtagtt | | |

M5

| | | | | | |
|---|---|---|---|---|---|
| F1 | 310 | F | tgccgccgcgcgcatggtcgtaatgggaccgatagc | 11593 | M3C1 |
| | | | ccgt | | |
| | 311 | R | tgctggtccatgaagatgaa | | |
| F2 | 312 | F | tcgagctgatgtttgacgac | 8884 | M3C1 |
| | 313 | R | tcatcaggccggttctgacctggtgagggtaggggg | | |
| | | | atatcgctatcccctccccttacc | | |
| F3 | 314 | F | ggtaaggggaggggatagcgatatccccctaccctc | 3675 | M3C1 |
| | | | accaggtcagaaccggcctgatga | | |
| | 315 | R | cattgcaaagcgactgatgt | | |
| F4 | 316 | F | gatccgctccttgaactctg | 6253 | M3C1 |
| | 317 | R | aggcccttgccaatgaat | | |
| F5 | 318 | F | ttctttgaatgcgcgggcgtcctggtgagcgtagtcca | 10447 | M3C1 |
| | | | gc | | |
| | 319 | R | agccctcccgtatcgtagtt | | |

M6

| | | | | | |
|---|---|---|---|---|---|
| F1 | 320 | F | aaaacaaaagcccggaaaccgggctttcgtctcttgc | 12900 | M3C1 |
| | | | cgccggggttgatcctccggttg | | |
| | 321 | R | tgctggtccatgaagatgaa | | |
| F2 | 322 | F | tcgagctgatgtttgacgac | 11800 | M3C1 |
| | 323 | R | tcatcaggccggttctgacctggtgagggtaggggg | | |
| | | | atatcgctatcccctccccttacc | | |
| F3 | 324 | F | ggtaaggggaggggatagcgatatccccctaccctc | 3675 | M3C1 |
| | | | accaggtcagaaccggcctgatga | | |
| | 325 | R | cattgcaaagcgactgatgt | | |
| F4 | 326 | F | gatccgctccttgaactctg | 6253 | M3C1 |
| | 327 | R | aggcccttgccaatgaat | | |
| F5 | 328 | F | ttctttgaatgcgcgggcgtcctggtgagcgtagtcca | 4262 | M3C1 |
| | | | gc | | |
| | 329 | R | gggcacttcgcccaggtcagcaaccggaggatcaac | | |
| | | | cccggcggcaagagacgaaagccc | | |

M7

| | | | | | |
|---|---|---|---|---|---|
| F1 | 330 | F | tgccgccgcgcgcatggtcgtaatgggaccgatagc | 5132 | M3C1 |
| | | | ccgt | | |
| | 331 | R | tgccccggcgtgagtcggggcaatcccgcaaggag | | |
| | | | ggtgaccgcttgccctcatctgtta | | |
| F2 | 332 | F | ctggccggctaccgccggcgtaacagatgagggca | 3819 | M3C1 |
| | | | agcggtcaccctccttgcgggattg | | |
| | 333 | R | tgctggtccatgaagatgaa | | |
| F3 | 334 | F | tcgagctgatgtttgacgac | 8884 | M3C1 |
| | 335 | R | tcatcaggccggttctgacctggtgagggtaggggg | | |
| | | | atatcgctatcccctccccttacc | | |
| F4 | 336 | F | ggtaaggggaggggatagcgatatccccctaccctc | 3675 | M3C1 |
| | | | accaggtcagaaccggcctgatga | | |
| | 337 | R | cattgcaaagcgactgatgt | | |
| F5 | 338 | F | gatccgctccttgaactctg | 6253 | M3C1 |
| | 339 | R | aggcccttgccaatgaat | | |
| F6 | 340 | F | ttctttgaatgcgcgggcgtcctggtgagcgtagtcca | 10447 | M3C1 |
| | | | gc | | |
| | 341 | R | agccctcccgtatcgtagtt | | |

M8

| | | | | | |
|---|---|---|---|---|---|
| F1 | 342 | F | aaaacaaaagcccggaaaccgggctttcgtctcttgc | 6610 | M3C1 |
| | | | cgccggggttgatcctccggttg | | |
| | 343 | R | tgccccggcgtgagtcggggcaatcccgcaaggag | | |
| | | | ggtgaccgcttgccctcatctgtta | | |
| F2 | 344 | F | ctggccggctaccgccggcgtaacagatgagggca | 11686 | M3C1 |
| | | | agcggtcaccctccttgcgggattg | | |
| | 345 | R | tgacctggtgagggtagggggatatcgctatcccctc | | |
| | | | cccggtcgtagctcctaagagtc | | |
| F3 | 346 | F | agcccgcccaatcctgaaacgactcttaggagctacg | 3710 | M3C1 |
| | | | accggggaggggatagcgatatc | | |
| | 347 | R | cattgcaaagcgactgatgt | | |
| F4 | 348 | F | gatccgctccttgaactctg | 6253 | M3C1 |
| | 349 | R | aggcccttgccaatgaat | | |
| F5 | 350 | F | ttctttgaatgcgcgggcgtcctggtgagcgtagtcca | 4262 | M3C1 |
| | | | gc | | |
| | 351 | R | gggcacttcgcccaggtcagcaaccggaggatcaac | | |
| | | | cccggcggcaagagacgaaagccc | | |

M3C1_F1

| | | | | | |
|---|---|---|---|---|---|
| F1 | 352 | F | tgccgccgcgcgcatggtcgtaatgggaccgatagc | 11593 | M3C1 |
| | | | ccgt | | |
| | 353 | R | tgctggtccatgaagatgaa | | |
| F2 | 354 | F | tcgagctgatgtttgacgac | 11800 | M3C2 |
| | 355 | R | atcggcgtgaagcccaacagggcca | | |
| F3 | 356 | F | gtggacattggtttcagcaa | 6500 | M3C2 |
| | 357 | R | aagcgatgaatgatcccaag | | |
| F4 | 358 | F | tgtaacgcttcccggtagtc | 11797 | M3C2 |
| | 359 | R | aggcccttgccaatgaat | | |
| F5 | 360 | F | ttctttgaatgcgcgggcgtcctggtgagcgtagtcca | 10447 | M3C2 |
| | | | gc | | |
| | 361 | R | agccctcccgtatcgtagtt | | |

M3C1_F2

| | | | | | |
|---|---|---|---|---|---|
| F1 | 362 | F | tgccgccgcgcgcatggtcgtaatgggaccgatagc | 11593 | M3C2 |
| | | | ccgt | | |
| | 363 | R | tgctggtccatgaagatgaa | | |
| F2 | 364 | F | tcgagctgatgtttgacgac | 11800 | M3C1 |
| | 365 | R | atcggcgtgaagcccaacagggcca | | |
| F3 | 366 | F | gtggacattggtttcagcaa | 6500 | M3C2 |
| | 367 | R | aagcgatgaatgatcccaag | | |
| F4 | 368 | F | tgtaacgcttcccggtagtc | 11797 | M3C2 |
| | 369 | R | aggcccttgccaatgaat | | |
| F5 | 370 | F | ttctttgaatgcgcgggcgtcctggtgagcgtagtcca | 10447 | M3C2 |
| | | | gc | | |
| | 371 | R | agccccccgtatcgtagtt | | |

M3C1_F3

| | | | | | |
|---|---|---|---|---|---|
| F1 | 372 | F | tgccgccgcgcgcatggtcgtaatgggaccgatagc | 11593 | M3C2 |
| | | | ccgt | | |
| | 373 | R | tgctggtccatgaagatgaa | | |
| F2 | 374 | F | tcgagctgatgtttgacgac | 11800 | M3C2 |
| | 375 | R | atcggcgtgaagcccaacagggcca | | |
| F3 | 376 | F | gtggacattggtttcagcaa | 6500 | M3C1 |
| | 377 | R | aagcgatgaatgatcccaag | | |
| F4 | 378 | F | tgtaacgcttcccggtagtc | 11797 | M3C2 |
| | 379 | R | aggcccttgccaatgaat | | |
| F5 | 380 | F | ttctttgaatgcgcgggcgtcctggtgagcgtagtcca | 10447 | M3C2 |
| | | | gc | | |
| | 381 | R | agccctcccgtatcgtagtt | | |

M3C1_F4

| | | | | | |
|---|---|---|---|---|---|
| F1 | 382 | F | tgccgccgcgcgcatggtcgtaatgggaccgatagc | 11593 | M3C2 |
| | | | ccgt | | |
| | 383 | R | tgctggtccatgaagatgaa | | |
| F2 | 384 | F | tcgagctgatgtttgacgac | 11800 | M3C2 |
| | 385 | R | atcggcgtgaagcccaacagggcca | | |
| F3 | 386 | F | gtggacattggtttcagcaa | 6500 | M3C2 |
| | 387 | R | aagcgatgaatgatcccaag | | |
| F4 | 388 | F | tgtaacgcttcccggtagtc | 11797 | M3C1 |
| | 389 | R | aggcccttgccaatgaat | | |
| F5 | 390 | F | ttctttgaatgcgcgggcgtcctggtgagcgtagtcca | 10447 | M3C2 |
| | | | gc | | |
| | 391 | R | agccctcccgtatcgtagtt | | |

M3C1_F5

| | | | | | |
|---|---|---|---|---|---|
| F1 | 392 | F | tgccgccgcgcgcatggtcgtaatgggaccgatagc | 11593 | M3C2 |
| | | | ccgt | | |
| | 393 | R | tgctggtccatgaagatgaa | | |
| F2 | 394 | F | tcgagctgatgtttgacgac | 11800 | M3C2 |
| | 395 | R | atcggcgtgaagcccaacagggcca | | |
| F3 | 396 | F | gtggacattggtttcagcaa | 6500 | M3C2 |
| | 397 | R | aagcgatgaatgatcccaag | | |
| F4 | 398 | F | tgtaacgcttcccggtagtc | 11797 | M3C2 |
| | 399 | R | aggcccttgccaatgaat | | |
| F5 | 400 | F | ttctttgaatgcgcgggcgtcctggtgagcgtagtcca | 10447 | M3C1 |
| | | | gc | | |
| | 401 | R | agccctcccgtatcgtagtt | | |

pTA-Mob 2.0 Tp

| | | | | | |
|---|---|---|---|---|---|
| F1 | 402 | F | tgccgccgcgcgcatggtcgtaatgggaccgatagc | 5752 | pTA-Mob 2.0 |
| | | | ccgt | | |
| | 403 | R | tttaacctacttcctttggttccgggggatctcgcgactc | | |
| F2 | 404 | F | atcgaagagaagcaggacga | 6374 | pTA-Mob 2.0 |
| | 405 | R | tgctggtccatgaagatgaa | | |
| F3 | 406 | F | tcgagctgatgtttgacgac | 6137 | pTA-Mob2.0 |
| | 407 | R | ggacttgaggttgctctgct | | |
| F4 | 408 | F | ggaccaggcgcagtccaccatcaacggcctgatgag | 6000 | pTA-Mob 2.0 |
| | | | cgcc/ | | |
| | 409 | R | atcggcgtgaagcccaacagggcca | | |
| F5 | 410 | F | gtggacattggtttcagcaa | 6273 | pTA-Mob 2.0 |
| | 411 | R | agctcatgcatcacaacagc | | |
| F6 | 412 | F | gagcaatggatagccgatgt | 6206 | pTA-Mob 2.0 |
| | 413 | R | aagcgatgaatgatcccaag | | |
| F7 | 414 | F | tgtaacgcttcccggtagtc | 6295 | pTA-Mob 2.0 |
| | 415 | R | cattgcaaagcgactgatgt | | |
| F8a | 416 | F | gatccgctccttgaactctg | 1881 | pTA-Mob 2.0 |
| | 417 | R | atccaacggcgtcagccgagggcaagcggatggct | | |
| | | | gatgaaaccaagccaaccaggaagg | | |
| F8b | 418 | F | ccttcctggttggcttggtttcatcagccatccgcttgcc | 4435 | pTA-Mob 2.0 |
| | | | ctcggctgacgccgttggat | | |
| | 419 | R | aggcccttgccaatgaat | | |
| F9 | 420 | F | ttctttgaatgcgcgggcgtcctggtgagcgtagtcca | 6000 | pTA-Mob 2.0 |
| | | | gc | | |
| | 421 | R | cgttcccgcctgcccctgattggcccgctgatcgacc | | |
| | | | gct | | |
| F10 | 422 | F | aatgttgcaaggcgatcag | 5745 | pTA-Mob 2.0 |
| | 423 | R | agccctcccgtatcgtagtt | | |

pTA-Mob 2.0 To

| | | | | | |
|---|---|---|---|---|---|
| F1 | 424 | F | tgccgccgcgcgcatggtcgtaatgggaccgatagc | 5752 | pTA-Mob 2.0 |
| | | | ccgt | | |
| | 425 | R | tttaacctacttcctttggttccgggggatctcgcgactc | | |
| F2 | 426 | F | atcgaagagaagcaggacga | 6374 | pTA-Mob 2.0 |
| | 427 | R | tgctggtccatgaagatgaa | | |
| F3 | 428 | F | tcgagctgatgtttgacgac | 6137 | pTA-Mob2.0 |
| | 429 | R | ggacttgaggttgctctgct | | |
| F4 | 430 | F | ggaccaggcgcagtccaccatcaacggcctgatgag | 6000 | pTA-Mob 2.0 |
| | | | cgcc | | |
| | 431 | R | atcggcgtgaagcccaacagggcca | | |
| F5 | 432 | F | gtggacattggtttcagcaa | 6273 | pTA-Mob 2.0 |
| | 433 | R | agctcatgcatcacaacagc | | |
| F6 | 434 | F | gagcaatggatagccgatgt | 6206 | pTA-Mob 2.0 |
| | 435 | R | aagcgatgaatgatcccaag | | |
| F7 | 436 | F | tgtaacgcttcccggtagtc | 6295 | pTA-Mob 2.0 |
| | 437 | R | cattgcaaagcgactgatgt | | |
| F8a | 438 | F | gatccgctccttgaactctg | 1821 | pTA-Mob 2.0 |
| | 439 | R | gcagcccacctatcaaggtgtactgccttccagacga | | |
| | | | acgaagagcgattgaggaaaagg | | |
| F8b | 440 | F | taggccgacaggctcatgccggccgccgccgcctttt | 4526 | pTA-Mob 2.0 |
| | | | cctcaatcgctcttogttcgtct | | |
| | 441 | R | aggcccttgccaatgaat | | |
| F9 | 442 | F | ttctttgaatgcgcgggcgtcctggtgagcgtagtcca | 6000 | pTA-Mob 2.0 |
| | | | gc | | |
| | 443 | R | cgttcccgcctgcccctgattggcccgctgatcgacc | | |
| | | | gct | | |
| F10 | 444 | F | aatgttgcaaggcgatcag | 5745 | pTA-Mob 2.0 |
| | 445 | R | agccctcccgtatcgtagtt | | |

Sequencing primers to check mutations in traJ region

| | | | |
|---|---|---|---|
| F1 | 446 | F | gtttcagcaggccgcccagg |
| | 447 | R | cgctgcataaccctgcttcg |

pSC5

| | | | | | |
|---|---|---|---|---|---|
| F1 | 448 | F | tgccgccgcgcgcatggtcgtaatgggaccgatagc | 7280 | pTA-Mob 2.0- |
| | | | ccgt | | NAT |
| | 449 | R | tttaacctacttcctttggttccgggggatctcgcgactc | | |
| F2 | 450 | F | atcgaagagaagcaggacga | 6373 | M3C1 |
| | 451 | R | tgctggtccatgaagatgaa | | |
| F3 | 452 | F | tcgagctgatgtttgacgac | 11800 | M3C1 |
| | 453 | R | atcggcgtgaagcccaacagggcca | | |
| F4 | 454 | F | gtggacattggtttcagcaa | 3692 | M3C1 |
| | 455 | R | caagcattgggttccgtatctaaccatgaccgtgcttc | | |
| | | | gaaccactcggagggacggttt | | |
| F5 | 456 | F | aaaccgtccctccgagtggttcgaagcacggtcatgg | 2573 | pGMO1 |
| | | | ttagatacggaacccaatgcttg | | |
| | 457 | R | aggagaactagagcgtaattaccctgttatccctacaa | | |
| | | | acaccctttcaatgggcttcga | | |
| F6 | 458 | F | cattgaaagggtgtttgtagggataacagggtaattac | 3477 | M3C1 |
| | | | gctctagttctccttagtaggt | | |
| | 459 | R | aagcgatgaatgatoccaag | | |
| F7 | 460 | F | tgtaacgcttcccggtagtc | 11797 | M3C1 |
| | 461 | R | aggcccttgccaatgaat | | |
| F8 | 462 | F | ttctttgaatgcgcgggcgtcctggtgagcgtagtcca | 10447 | M3C1 |
| | | | gc | | |
| | 463 | R | cgttcccgcctgcccctgattggcccgctgatcgacc | | |
| | | | gct | | |
| F9 | 464 | F | aatgttgcaaggcgatcag | 5745 | MV3C1 |
| | 465 | R | agccccccgtatcgtagtt | | |

pSC5GGv1

| | | | | | |
|---|---|---|---|---|---|
| F1 | 466 | F | tgccgccgcgcgcatggtcgtaatgggaccgatagc | 6112 | pSC5 |
| | | | ccgt | | |
| | 467 | R | taattagcatttttcgctttattctgttgtcgagatcttct | | |
| | | | ccttgcaggttcaacaact | | |
| F2 | 468 | F | aagatctcgacaacagaataaagcgaaaaatgctaat | 7196 | pSC5 |
| | | | aatgcactaacactcaggcctc | | |
| | | | (To domesticate BsaI site) | | |
| | 469 | R | tgctggtccatgaagatgaa | | |
| F3 | 470 | F | tcgagctgatgtttgacgac | 6137 | pSC5 |
| | 471 | R | ggacttgaggttgctctgct | | |
| F4 | 472 | F | ggaccaggcgcagtccaccatcaacggcctgatgag | 6000 | pSC5 |
| | | | cgcc | | |
| | 473 | R | atcggcgtgaagcccaacagggcca | | |
| F5 | 474 | F | gtggacattggtttcagcaa | 11926 | pSC5 |
| | 475 | R | caatgtctgatgcaatatggacaattggtttcttggtctc | | |
| | | | attaccctgttatccctaca | | |
| F6 | 476 | F | accttcgggtgggcctttctgcgtttataggtctcatgct | 1187 | pSC5 |
| | | | tacgctctagttctccttag | | |
| | 477 | R | cgaattgaaacggagggcgacaagaagggcgaga | | |
| | | | agtcgggcttctacgteggccacctc | | |
| F7 | 478 | F | cgacttctcgcccttcttgtcgccctccgtttcaattcgg | 8239 | pSC5 |
| | | | tgcttcttgccgtccatga | | |
| | | | (To domesticate BsaI site) | | |
| | 479 | R | cattgcaaagcgactgatgt | | |
| F8 | 480 | F | gatccgctccttgaactctg | 6256 | pSC5 |
| | 481 | R | aggcccttgccaatgaat | | |
| F9 | 482 | F | aatgttgcaaggcgatcag | 4928 | pSC5 |
| | 483 | R | agccctcccgtatcgtagtt | | |
| F10 | 484 | F | attgaaagggtgtttgtagggataacagggtaatgag | 1276 | PAGE2.0-i |
| | | | accaagaaaccaattgtccatat | | |
| | | | (To amplify RFP Gene) | | |
| | 485 | R | Gagaacctactaaggagaactagagcgtaagcatga | | |
| | | | gacctataaacgcagaaaggccca | | |
| | | | (To amplify RFP Gene) | | |

Sh ble fragment for Golden Gate assembly

| | | | | | |
|---|---|---|---|---|---|
| Sh ble gene | 486 | F | ggtctcagtaatatcaagcttg | 1063 | pRS32 (From |
| | 487 | R | taggtctcaagcaactggatggcg | | Shapiro lab) |

HindII fragment for Golden Gate assembly

| | | | | | |
|---|---|---|---|---|---|
| HindII toxic | 488 | F | ggtctcagtaaggcgcgcccttggcagaacatatcca | 1618 | pUC57 HindII |
| | | | tcgcgtccgccatctccagcagc | | plasmid |
| gene | 489 | R | ggtctcagatttatcttcgtttcctgcaggtttttgttctgt | | (Synthesized |
| | | | gcagttgggttaagaata | | vector) |

A. laidlawii toxic gene fragments for Golden Gate assembly

| | | | | | |
|---|---|---|---|---|---|
| 1st half of Al | 490 | F | ggtctcagtaaggtaatgcttggcatgttcatatagatg | 2072 | A. laidlawii |
| toxic gene | | | gtttaacagatcattataaag | | strain PG-8A |
| | 491 | R | ggtctcatagtgcattaaatcctggaggcgttaatttact | | gDNA |
| | | | tctatgtgcttcaaaggctt | | |
| ACTI intron | 492 | F | ggtctcaactagtatgttctagcgcttgcaccatcccat | 327 | S. cerevisiae |
| | | | ttaactgtaagaagaattgca | | gDNA |
| | 493 | R | ggtctcactaaacatataatatagcaacaaaaagaatg | | |
| | | | aagcaatcgatgttagtacatg | | |
| 2nd half of Al | 494 | F | ggtctcattagtctaatgaaggtttacttcaatattacgct | 1061 | A. laidlawii |
| toxic gene | | | tcattaaatggtttaactg | | strain PG-8A |
| | 495 | R | ggtctcaagcaggaccataagaagtccgaaaaactat | | gDNA |
| | | | taatctgtccaaaatgtttaata | | |

Genotyping NAT marker

| | | | | |
|---|---|---|---|---|
| NAT gene | 496 | F | tccagttgatccaccattga | 283 |
| | 497 | R | caaccacaaatgaccagcac | |

pSC5GGv2

| | | | | | |
|---|---|---|---|---|---|
| F1 | 498 | F | tgccgccgcgcgcatggtcgtaatgggaccgatagc | 4235 | pSC5GGv1 C8 |
| | | | ccgt | | |
| | 499 | R | Atatggacaattggtttcttggtctcattacatatataca | | |
| | | | catgtatatatatcgtatgc | | |
| F2 | 500 | F | tgggcctttctgcgtttataggtctcatgctgtatacctat | 3149 | pSC5GGv1 C8 |
| | | | gaatgtcagtaagtatgta | | |
| | 501 | R | tttaacctacttcctttggttccgggggatctcgcgactc | | |
| F3 | 502 | F | taggagtgcggttggaacgt | 6177 | pSC5GGv1 C8 |
| | 503 | R | tgctggtccatgaagatgaa | | |
| F4 | 504 | F | tcgagctgatgtttgacgac | 6137 | pSC5GGv1 C8 |
| | 505 | R | ggacttgaggttgctctgct | | |
| F5 | 506 | F | ggaccaggcgcagtccaccatcaacggcctgatgag | 6000 | pSC5GGv1 C8 |
| | | | cgcc | | |
| | 507 | R | atcggcgtgaagcccaacagggcca | | |
| F6 | 508 | F | gtggacattggtttcagcaa | 6300 | pSC5GGv1 C8 |
| | 509 | R | aggagaactagagcgtaattaccctgttatccctacaa | | |
| | | | acaccctttcaatgggcttcga | | |
| F7 | 510 | F | cattgaaagggtgtttgtagggataacagggtaattac | 3512 | pSC5GGv1 C8 |
| | | | gctctagttctccttagtaggt | | |
| | 511 | R | aagcgatgaatgatcccaag | | |
| F8 | 512 | F | tgtaacgcttcccggtagtc | 6295 | pSC5GGv1 C8 |
| | 513 | R | cattgcaaagcgactgatgt | | |
| F9 | 514 | F | gatccgctccttgaactctg | 6253 | pSC5GGv1 C8 |
| | 515 | R | aggcccttgccaatgaat | | |
| F10 | 516 | F | ttctttgaatgcgcgggcgtcctggtgagcgtagtcca | 6000 | pSC5GGv1 C8 |
| | | | gc | | |
| | 517 | R | cgttcccgcctgcccctgattggcccgctgatcgacc | | |
| | | | gct | | |
| F11 | 518 | F | aatgttgcaaggcgatcag | 4928 | pSC5GGv1 C8 |
| | 519 | R | agccctcccgtatcgtagtt | | |
| F12 | 520 | F | gcatacgatatatatacatgtgtatatatgtaatgagac | 1276 | pAGE2.0-i |
| | | | caagaaaccaattgtccatat | | |
| | | | (To amplify RFP Gene) | | |
| | 521 | R | tacatacttactgacattcataggtatacagcatgagac | | |
| | | | ctataaacgcagaaaggccca | | |
| | | | (To amplify RFP Gene) | | |

pTA-Mob 2.1

| | | | | | |
|---|---|---|---|---|---|
| F1 | 522 | F | tgccgccgcgcgcatggtcgtaatgggaccgatagc | 5259 | pTA-Mob 2.0 |
| | | | ccgt | | |
| | 523 | R | gggagtatctggctgggccaacgttccaaccgcactc | | |
| | | | ctaccggccagcctcgcagagca | | |
| F2 | 524 | F | gcggtgctcaacgggaatcctgctctgcgaggctgg | 6177 | pTA-Mob 2.0 |
| | | | ccggtaggagtgcggttggaacgt | | |
| | 525 | R | tgctggtccatgaagatgaa | | |
| F3 | 526 | F | tcgagctgatgtttgacgac | 6137 | pTA-Mob 2.0 |
| | 527 | R | ggacttgaggttgctctgct | | |
| F4 | 528 | F | ggaccaggcgcagtccaccatcaacggcctgatgag | 6000 | pTA-Mob 2.0 |
| | | | cgcc | | |
| | 529 | R | atcggcgtgaagcccaacagggcca | | |
| F5 | 530 | F | gtggacattggtttcagcaa | 6289 | pTA-Mob 2.0 |
| | 531 | R | agctcatgcatcacaacagc | | |
| F6 | 532 | F | gagcaatggatagccgatgt | 6204 | pTA-Mob 2.0 |
| | 533 | R | aagcgatgaatgatcccaag | | |
| F7 | 534 | F | tgtaacgcttcccggtagtc | 6295 | pTA-Mob 2.0 |
| | 535 | R | cattgcaaagcgactgatgt | | |
| F8 | 536 | F | gatccgctccttgaactctg | 6256 | pTA-Mob 2.0 |
| | 537 | R | aggcccttgccaatgaat | | |
| F9 | 538 | F | ttctttgaatgcgcgggcgtcctggtgagcgtagtcca | 6000 | pTA-Mob 2.0 |
| | | | gc | | |
| | 539 | R | cgttcccgcctgcccctgattggcccgctgatcgacc | | |
| | | | gct | | |
| F10 | 540 | F | aatgttgcaaggcgatcag | 4928 | pTA-Mob 2.0 |
| | 541 | R | agccctcccgtatcgtagtt | | |

pSC5.1

| | | | | | |
|---|---|---|---|---|---|
| F1 | 542 | F | tgccgccgcgcgcatggtcgtaatgggaccgatagc | 6757 | pSC5 |
| | | | ccgt | | |
| | 543 | R | cacgcgacaagcacgagcgagatatoccaatcaagc | | |
| | | | tagtccggccagcctcgcagagca | | |
| F2 | 544 | F | gcggtgctcaacgggaatcctgctctgcgaggctgg | 6177 | pSC5 |
| | | | ccggtaggagtgcggttggaacgt | | |
| | 545 | R | tgctggtccatgaagatgaa | | |
| F3 | 546 | F | tcgagctgatgtttgacgac | 6137 | pSC5 |
| | 547 | R | ggacttgaggttgctctgct | | |
| F4 | 548 | F | ggaccaggcgcagtccaccatcaacggcctgatgag | 6000 | pSC5 |
| | | | cgcc | | |
| | 549 | R | atcggcgtgaagcccaacagggcca | | |
| F5 | 550 | F | gtggacattggtttcagcaa | 9812 | pSC5 |
| | 551 | R | aagcgatgaatgatcccaag | | |
| F6 | 552 | F | tgtaacgcttcccggtagtc | 6295 | pSC5 |
| | 553 | R | cattgcaaagcgactgatgt | | |
| F7 | 554 | F | gatccgctccttgaactctg | 6256 | pSC5 |
| | 555 | R | aggcccttgccaatgaat | | |
| F8 | 556 | F | ttctttgaatgcgcgggcgtcctggtgagcgtagtcca | 10549 | pSC5 |
| | | | gc | | |
| | 557 | R | agccctcccgtatcgtagtt | | |

qPCR primers

| | | | | | |
|---|---|---|---|---|---|
| rrsA gene | 558 | F | ctcttgccatcggatgtgccca | 106 | pTA-Mob 2.1 |
| | 559 | R | ccagtgtggctggtcatcctctca | | and pSC5.1 |
| | | | | | DNA |
| cysG gene | 560 | F | ttgtcggcggtggtgatgtc | 136 | pTA-Mob 2.1 |
| | | | | | and pSC5.1 |
| | 561 | R | atgcggtgaactgtggaataaacg | | CDNA |
| traJ gene | 562 | F | acgacgcccgtgattttgtag | 109 | pTA-Mob 2.1 |
| | | | | | and pSC5.1 |
| | 563 | R | gccttccagacgaacgaaga | | CDNA |

TABLE 5
Whole plasmid sequencing of minimal conjugative plasmid 3 (M3C1 and M3C2).

| | | Plasmid | Nucleotide | Amino Acid | |
|---|---|---|---|---|---|
| Plasmid | Fragment | position | Mutation | Mutation | Gene |
| M3C1 | Fragment 1 | 3,342 | 1 bp insertion (T) | | |
| M3C1 | Fragment 2 | 14,111 | G₇₃₁ → T | R₂₄₄ → L | trbF |
| M3C1 | Fragment 2 | 20,676 | G₅₀₅ → T | D₁₆₉ → Y | trbN |
| M3C1 | Fragment 3 | 25,788 | C₁₃₈ → A | | |
| M3C1 | Fragment 3 | 26,489 | 4,912 bp Deletion | | URA3 |
| M3C1 | Fragment 4 | 37,156 | T₇₇ → G | E₂₆ → A | traJ |
| M3C1 | Fragment 4 | 37,242-37,246 | GAATT → CTCGG | | |
| M3C2 | Fragment 3 | 25,787 | C₁₃₈ → A | | parE |
| M3C2 | Fragment 3 | 26,488 | 4,912 bp Deletion | | URA3 |
| M3C2 | Fragment 4 | 37,458 | G → T | | |
| M3C2 | Fragment 5 | 48,347 | A₄₇ → G | L₁₆ → S | klaA |

Mutations in M3C1 and M3C2 identified by next-generation sequencing and alignment to the reference sequence: pTA-Mob 2.0. Nucleotide numbering begins at the forward primers of Fragment 1.
Note:
The 4,912 bp is designed deletion.

TABLE 6
Cis- and trans- conjugation of super conjugative plasmid (pSC5).

| | | Rep 1 | Rep 2 | Rep 3 | Rep 4 | Average |
|---|---|---|---|---|---|---|
| Configuration | Plasmid | (CFU mL⁻¹) | (CFU mL⁻¹) | (CFU mL⁻¹) | (CFU mL⁻¹) | (CFU mL⁻¹) |
| Cis | pTA-Mob 2.0 | 3.1 × 10³ | 4.2 × 10³ | 3.6 × 10³ | 3.8 × 10³ | 3.7 × 10³ |
| (S. cerevisiae) | pSC5 | 1.9 × 10⁶ | 1.3 × 10⁵ | 1.3 × 10⁵ | 1.0 × 10⁵ | 1.4 × 10⁵ |
| Trans | pTA-Mob 2.0 | 6.6 × 10² | 5.7 × 10² | 1.3 × 10³ | 2.8 × 10³ | 1.3 × 10³ |
| (S. cerevisiae) | pSC5 | 5.3 × 10⁴ | 8.3 × 10⁴ | 8.5 × 10⁴ | 6.2 × 10⁴ | 7.1 × 10⁴ |
| Cis | pTA-Mob 2.0 | 2.2 × 10⁶ | 1.0 × 10⁶ | 2.2 × 10⁶ | 2.7 × 10⁶ | 2.1 × 10⁶ |
| (E. coli) | pSC5 | 1.0 × 10⁶ | 1.4 × 10⁶ | 1.0 × 10⁶ | 1.2 × 10⁶ | 1.2 × 10⁶ |

S. cerevisiae transconjugant concentrations following bacterial conjugation of pSC5 compared to pTA-Mob 2.0 in either cis- (self-transmissible) or trans- (mobilization of a secondary plasmid - pAGE2.0.T) and E. coli transconjugant colony counts following bacterial conjugation of pSC5 compared to pTA-Mob 2.0 in cis- from E. coli (FIG. 4).
Results are shown as colony-forming units per mL (CFU mL⁻¹) for four biological replicates each with two technical replicates.

TABLE 7
Recipient yeast cell concentrations used in conjugation experiments of FIG. 4.

| | | Rep 1 | Rep 2 | Rep 3 | Rep 4 | Average |
|---|---|---|---|---|---|---|
| Configuration | Plasmid | (CFU mL⁻¹) | (CFU mL⁻¹) | (CFU mL⁻¹) | (CFU mL⁻¹) | (CFU mL⁻¹) |
| Cis | pTA-Mob 2.0 | 1.3 × 10⁷ | 1.5 × 10⁷ | 3.9 × 10⁷ | 4.7 × 10⁷ | 2.9 × 10⁷ |
| | pSC5 | 6.7 × 10⁷ | 6.9 × 10⁷ | 1.1 × 10⁸ | 1.1 × 10⁸ | 9.1 × 10⁷ |
| Trans | pTA-Mob 2.0 | 8.0 × 10⁶ | 4.0 × 10⁶ | 4.3 × 10⁷ | 3.7 × 10⁷ | 2.3 × 10⁷ |
| | pSC5 | 1.2 × 10⁷ | 1.5 × 10⁷ | 9.5 × 10⁷ | 9.3 × 10⁷ | 5.4 × 10⁷ |

Counts of S. cerevisiae colonies formed on the non-selective plate (1 × YPAD supplemented with ampicillin 100 μg mL⁻¹) following the bacterial conjugation of pSC5 and pTA-Mob 2.0 in cis- and trans- configuration.
Colony-forming units are presented per mL (CFU mL⁻¹) for four biological replicates each with two technical replicates.

TABLE 8
S. cerevisiae cell viability following conjugation with different E. coli strains.

| | Hemocytometer Cell Count | Colony Count (CFU mL⁻¹) | |
|---|---|---|---|
| Donor (D)/Recipient (R) | (Cells mL⁻¹) | Technical replicas | Average |
| D: no E. coli | 5.08 × 10⁸ | 2.01 × 10⁸ | 2.08 × 10⁸ |
| R: S. cerevisiae | | 2.27 × 10⁸ | |
| | | 1.97 × 10⁸ | |
| D: E. coli Epi300 | 6.14 × 10⁸ | 2.05 × 10⁸ | 2.19 × 10⁸ |
| R: S. cerevisiae | | 2.37 × 10⁸ | |
| | | 2.15 × 10⁸ | |
| D: E. coli Epi300 | 2.56 × 10⁸ | 1.4 × 10⁷ | 1.63 × 10⁷ |
| with pTA-Mob 2.0 | | 1.0 × 10⁷ | |
| R: S. cerevisiae | | 2.5 × 10⁷ | |
| D: E. coli Epi300 | 4.06 × 10⁸ | 1.41 × 10⁸ | 1.44 × 10⁸ |
| with pSC5 | | 1.45 × 10⁸ | |
| R: S. cerevisiae | | 1.47 × 10⁸ | |

S. cerevisiae cell concentrations obtained by hemocytometer (all cells) or plating on non-selective (1 × YPAD supplemented with ampicillin 100 μg mL⁻¹) plates (live cells) following a 3-hour incubation at 30 °C alone or with E. coli either harboring no plasmid, pTA-Mob 2.0, or pSC5.

TABLE 9
S. cerevisiae cell viability following conjugation with different S. meliloti strains.

| | Hemocytometer Cell Count (Cells mL⁻¹) | | Colony Count (CFU mL⁻¹) | |
|---|---|---|---|---|
| | Biological | | Biological | |
| Donor (D)/Recipient (R) | replicas | Average | replicas | Average |
| D: no S. meliloti | 3.04 × 10⁸ | 3.04 × 10⁸ | 1.27 × 10⁸ | 1.27 × 10⁸ |
| R: S. cerevisiae | | | | |
| D: S. meliloti Rm4126 | 3.16 × 10⁸ | 3.07 × 10⁸ | 9.70 × 10⁷ | 8.47 × 10⁷ |
| R⁻ with pTA-Mob 2.0 | 3.48 × 10⁸ | | 1.10 × 10⁸ | |
| R: S. cerevisiae | 2.58 × 10⁸ | | 4.70 × 10⁷ | |
| D: S. meliloti Rm4126 | 3.84 × 10⁸ | 4.11 × 10⁸ | 1.85 × 10⁸ | 1.60 × 10⁸ |
| R⁻ with pSC5 | 4.52 × 10⁸ | | 1.67 × 10⁸ | |
| R: S. cerevisiae | 3.98 × 10⁸ | | 1.27 × 10⁸ | |

S. cerevisiae cell counts by hemocytometer (all cells) or plating on non-selective (1 × YPAD supplemented with ampicillin 100 μg mL⁻¹) plates (live cells) following a 3-hour incubation at 30 °C alone or with S. meliloti either harboring pTA-Mob 2.0 or pSC5.

TABLE 10
S. cerevisiae transconjugant colony count
following conjugation with S. meliloti.

Colony Count (CFU)

| | Plasmid | 100 μL | 50 μL |
|---|---|---|---|
| Experiment | pTA-Mob 2.0 #1 | 114 | 53 |
| 1 | pTA-Mob 2.0 #2 | 108 | 34 |
| | pTA-Mob 2.0 #3 | 92 | 46 |
| | pSC5 #1 | 2514 | 1341 |
| | pSC5 #2 | 2399 | 1254 |
| | pSC5 #3 | 2504 | 1220 |
| Experiment | pTA-Mob 2.0 #1 | 147 | 68 |
| 2 | pTA-Mob 2.0 #2 | 123 | 54 |
| | pTA-Mob 2.0 #3 | 87 | 39 |
| | pSC5 #1 | 1044 | 546 |
| | pSC5 #2 | 2145 | 1159 |
| | pSC5 #3 | 572 | 260 |

Colony counts of S. cerevisiae transconjugant re-suspension (2 mL) following bacterial conjugation with three biological replicates of S. meliloti (Rm4126) harboring either pTA-Mob 2.0 or pSC5, plated on complete synthetic yeast media lacking histidine and supplemented with ampicillin (100 μg mL⁻¹) (FIG. 9).

TABLE 11
Yeast transconjugant colony counts for the conjugation-
based antifungal experiment (FIG. 7).

| | | Yeast Selection Marker (CFU) | | | | | | | | |
|---|---|---|---|---|---|---|---|---|---|---|
| | | HIS3 | | | | TRP1 | | | | Ratio |
| Plasmid | | Rep 1 | Rep 2 | Rep 3 | Average | Rep 1 | Rep 2 | Rep 3 | Average | HIS3/TRP1 |
| 1 X | pSC5 | tmtc | tmtc | 592 | 592.00 | tmtc | tmtc | 302 | 302.0 | 1.960 |
| | pSC5-toxic1 | tmtc | tmtc | 230 | 230.00 | tmtc | tmtc | 264 | 264.0 | 0.871 |
| | pSC5-toxic2 | 25 | 17 | 5 | 15.67 | 594 | tmtc | 169 | 381.5 | 0.041 |
| | pSC5-toxic3 | 2 | 1 | 0 | 1.00 | 428 | tmtc | 188 | 308.0 | 0.003 |
| 10 X | pSC5 | 208 | 436 | 55 | 233 | 174 | 262 | 33 | 156.3 | 1.490 |
| | pSC5-toxic1 | 62 | 105 | 27 | 64.7 | 107 | 149 | 23 | 93 | 0.695 |
| | pSC5-toxic2 | 4 | 1 | 0 | 1.7 | 66 | 72 | 18 | 52 | 0.032 |
| | pSC5-toxic3 | 0 | 0 | 0 | 0 | 50 | 107 | 17 | 58 | 0 |

S. cerevisiae transconjugant colony counts following bacterial conjugation with E. coli harboring pAGE2.0.T and either pSC5, pSC5-toxic1, pSC5-toxic2, or pSC5-toxic3.
Note:
Colonies were counted from plating 100 μL of undiluted (1 X) and diluted (10 X) yeast transconjugant re-suspension (2 mL) on synthetic yeast media lacking either histidine or tryptophan.
Colonies were counted manually.
tmtc—too many to count.

Sequence Listing
traJ (wild type)
Underlined - traJ promoter region
Non-underlined - traJ open reading frame
Bolded - wild type partial promoter region - does not improve conjugation from bacteria to
eukaryotic cell
SEQ ID NO: 1
cgtctctcgcctgtcccctcagttcagtaatttcctgcatttgcctgtttccagtcggtagatattccacaaaacagcagggaagcagcgcttttc
cgctgcataaccctgcttcggggtcattatagcgattttttcggtatatccatcctttttcgcacgatatacaggattttgccaaagggttcgtgta
gactttccttggtgtatccaacggcgtcagaattcggcaagcggatggctgatgaaaccaagccaaccaggaagggcagcccacctatca
aggtgtactgccttccagacgaacgaagagagattgaggaaaaggcggcggcggccggcatgagcctgtcggcctacctgctggccgtc
ggccagggctacaaaatcacgggcgtcgtggactatgagcacgtccgcgagctggcccgcatcaatggcgacctgggccgcctgggcg
gcctgctgaaactctggctcaccgacgacccgcgcacggcgcggttcggtgatgccacgatcctcgccctgctggcgaagatcgaagag
aagcaggacgagcttggcaaggtcatgatgggcgtggtccgcccgagggcagagccatga
traJ (mutated)
Underlined - traJ promoter region
Non-underlined - traJ open reading frame
Bolded - mutated partial promoter region responsible for conjugation improvement from bacteria
to eukaryotic cell
SEQ ID NO: 2
cgtctctcgcctgtcccctcagttcagtaatttcctgcatttgcctgtttccagtcggtagatattccacaaaacagcagggaagcagcgcttttc
cgctgcataaccctgcttcggggtcattatagcgattttttcggtatatccatcctttttcgcacgatatacaggattttgccaaagggttcgtgta
gactttccttggtgtatccaacggcgtcagccgagggcaagcggatggctgatgaaaccaagccaaccaggaagggcagcccacctatc
aaggtgtactgccttccagacgaacgaagagagattgaggaaaaggcggcggcggccggcatgagcctgtcggcctacctgctggccgt
cggccagggctacaaaatcacgggcgtcgtggactatgagcacgtccgcgagctggcccgcatcaatggcgacctgggccgcctgggc
ggcctgctgaaactctggctcaccgacgacccgcgcacggcgcggttcggtgatgccacgatcctcgccctgctggcgaagatcgaaga
gaagcaggacgagcttggcaaggtcatgatgggcgtggtccgcccgagggcagagccatga
SEQ ID NO: 3: ccgag

### REFERENCES

(1) Fisher, M. C., Gurr, S. J., Cuomo, C. A., Blehert, D. S., Jin, H., Stukenbrock, E. H., Stajich, J. E., Kahmann, R., Boone, C., Denning, D. W., Gow, N. A. R., Klein, B. S., Kronstad, J. W., Sheppard, D. C., Taylor, J. W., Wright, G. D., Heitman, J., Casadevall, A., and Cowen, L. E. (2020) Threats posed by the fungal kingdom to humans, wildlife, and agriculture. MBio. 11, e00449-20.
(2) Hyde, K. D., Xu, J., Rapior, S., Jeewon, R., Lumyong, S., Niego, A. G. T., Abeywickrama, P. D., Aluthmuhandiram, J. V. S., Brahamanage, R. S., Brooks, S., Chaiyasen, A., Chethana, K. W. T., Chomnunti, P., Chepkirui, C., Chuankid, B., de Silva, N. I., Doilom, M., Faulds, C., Gentekaki, E., Gopalan, V., Kakumyan, P., Harishchandra, D., Hemachandran, H., Hongsanan, S., Karunarathna, A., Karunarathna, S. C., Khan, S., Kumla, J., Jayawardena, R. S., Liu, J.-K., Liu, N., Luangharn, T., Macabeo, A. P. G., Marasinghe, D. S., Meeks, D., Mortimer, P. E., Mueller, P., Nadir, S., Nataraja, K. N., Nontachaiyapoom, S., O'Brien, M., Penkhrue, W., Phukhamsakda, C., Ramanan, U. S., Rathnayaka, A. R., Sadaba, R. B., Sandargo, B., Samarakoon, B. C., Tennakoon, D. S., Siva, R., Sriprom, W., Suryanarayanan, T. S., Sujarit, K., Suwannarach, N., Suwunwong, T., Thongbai, B., Thongklang, N., Wei, D., Wijesinghe, S. N., Winiski, J., Yan, J., Yasanthika, E., and Stadler, M. (2019) The amazing potential of fungi: 50 ways we can exploit fungi industrially. Fungal Divers. 97, 1-136.
(3) Parapouli, M., Vasileiadis, A., Afendra, A. S., and Hatziloukas, E. (2020) Saccharomyces cerevisiae and its industrial applications. AIMS Microbiol. 6, 1-31.
(4) Edwards-Ingram, L., Gitsham, P., Burton, N., Warhurst, G., Clarke, I., Hoyle, D., Oliver, S. G., and Stateva, L. (2007) Genotypic and physiological characterization of Saccharomyces boulardii, the probiotic strain of Saccharomyces cerevisiae. Appl. Environ. Microbiol. 73, 2458-2467.
(5) Everard, A., Matamoros, S., Geurts, L., Delzenne, N. M., and Cani, P. D. (2014) Saccharomyces boulardii administration changes gut microbiota and reduces hepatic steatosis, low-grade inflammation, and fat mass in obese and type 2 diabetic db/db mice. MBio. 5, e01011-14.
(6) Pappas, P. G., Lionakis, M. S., Arendrup, M. C., Ostrosky-Zeichner, L., and Kullberg, B. J. (2018) Invasive candidiasis. Nature Reviews Disease Primers. 4, 1-20.
(7) Limon, J. J., Tang, J., Li, D., Wolf, A. J., Michelsen, K. S., Funari, V., Gargus, M., Nguyen, C., Sharma, P., Maymi, V. I., Iliev, I. D., Skalski, J. H., Brown, J., Landers, C., Borneman, J., Braun, J., Targan, S. R., McGovern, D. P. B., and Underhill, D. M. (2019) Malassezia is associated with Crohn's disease and exacerbates colitis in mouse models. Cell Host Microbe. 25, 377-388.e6.
(8) Aykut, B., Pushalkar, S., Chen, R., Li, Q., Abengozar, R., Kim, J. I., Shadaloey, S. A., Wu, D., Preiss, P., Verma, N., Guo, Y., Saxena, A., Vardhan, M., Diskin, B., Wang, W., Leinwand, J., Kurz, E., Kochen Rossi, J. A., Hundeyin, M., Zambrinis, C., Li, X., Saxena, D., and Miller, G. (2019) The fungal mycobiome promotes pancreatic oncogenesis via activation of MBL. Nature. 574, 264-267.
(9) Proctor, D. M., Dangana, T., Sexton, D. J., Fukuda, C., Yelin, R. D., Stanley, M., Bell, P. B., Baskaran, S., Deming, C., Chen, Q., Conlan, S., Park, M., NISC Comparative Sequencing Program, Welsh, R. M., Vallabhaneni, S., Chiller, T., Forsberg, K., Black, S. R., Pacilli, M., Kong, H. H., Lin, M. Y., Schoeny, M. E., Litvintseva, A. P., Segre, J. A., and Hayden, M. K. (2021) Integrated genomic, epidemiologic investigation of Candida auris skin colonization in a skilled nursing facility. Nat. Med. 27, 1401-1409.
(10) Du, H., Bing, J., Hu, T., Ennis, C. L., Nobile, C. J., and Huang, G. (2020) Candida auris: Epidemiology, biology, antifungal resistance, and virulence. PLOS Pathog. 16, e1008921.
(11) Geddes-McAlister, J., and Shapiro, R. S. (2019) New pathogens, new tricks: emerging, drug-resistant fungal pathogens and future prospects for antifungal therapeutics. Ann. N. Y. Acad. Sci. 1435, 57-78.
(12) Palacios Araya, D., Palmer, K. L., and Duerkop, B. A. (2021) CRISPR-based antimicrobials to obstruct antibiotic-resistant and pathogenic bacteria. PLOS Pathog. 17, e1009672.
(13) Citorik, R. J., Mimee, M., and Lu, T. K. (2014) Sequence-specific antimicrobials using efficiently delivered RNA-guided nucleases. Nat. Biotechnol. 32, 1141-1145.
(14) Kiga, K., Tan, X.-E., Ibarra-Chávez, R., Watanabe, S., Aiba, Y., Sato′o, Y., Li, F.-Y., Sasahara, T., Cui, B., Kawauchi, M., Boonsiri, T., Thitiananpakorn, K., Taki, Y., Azam, A. H., Suzuki, M., Penadés, J. R., and Cui, L. (2020) Development of CRISPR-Cas13a-based antimicrobials capable of sequence-specific killing of target bacteria. Nat. Commun. 11, 2934.
(15) Chavez, A., Pruitt, B. W., Tuttle, M., Shapiro, R. S., Cecchi, R. J., Winston, J., Turczyk, B. M., Tung, M., Collins, J. J., and Church, G. M. (2018) Precise Cas9 targeting enables genomic mutation prevention. Proc. Natl. Acad. Sci. U.S.A 115, 3669-3673.
(16) Gomaa, A. A., Klumpe, H. E., Luo, M. L., Selle, K., Barrangou, R., and Beisel, C. L. (2014) Programmable removal of bacterial strains by use of genome-targeting CRISPR-Cas systems. mBio. 5, e00928-13.
(17) Selle, K., Fletcher, J. R., Tuson, H., Schmitt, D. S., McMillan, L., Vridhambal, G. S., Rivera, A. J., Montgomery, S. A., Fortier, L.-C., Barrangou, R., Theriot, C. M., and Ousterout, D. G. (2020) In vivo targeting of Clostridioides difficile using phage-delivered CRISPR-Cas3 antimicrobials. MBio. 11, e00019-20.
(18) Bikard, D., Euler, C. W., Jiang, W., Nussenzweig, P. M., Goldberg, G. W., Duportet, X., Fischetti, V. A., and Marraffini, L. A. (2014) Exploiting CRISPR-Cas nucleases to produce sequence-specific antimicrobials. Nat. Biotechnol. 32, 1146-1150.
(19) Hamilton, T. A., Pellegrino, G. M., Therrien, J. A., Ham, D. T., Bartlett, P. C., Karas, B. J., Gloor, G. B., and Edgell, D. R. (2019) Efficient inter-species conjugative transfer of a CRISPR nuclease for targeted bacterial killing. Nat. Commun. 10, 4544.
(20) López-Igual, R., Bernal-Bayard, J., Rodríguez-Patón, A., Ghigo, J.-M., and Mazel, D. (2019) Engineered toxin-intein antimicrobials can selectively target and kill antibiotic-resistant bacteria in mixed populations. Nat. Biotechnol. 37, 755-760.
(21) Neil, K., Allard, N., Roy, P., Grenier, F., Menendez, A., Burrus, V., and Rodrigue, S. (2021) High-efficiency delivery of CRISPR-Cas9 by engineered probiotics enables precise microbiome editing. Mol. Syst. Biol. 17, e10335.
(22) Neil, K., Allard, N., Grenier, F., Burrus, V., and Rodrigue, S. (2020) Highly efficient gene transfer in the mouse gut microbiota is enabled by the Incl 2 conjugative plasmid TP114. Communications biology. 3, 1-9.
(23) Rodrigues, M., McBride, S. W., Hullahalli, K., Palmer, K. L., and Duerkop, B. A. (2019) Conjugative delivery of CRISPR-Cas9 for the selective depletion of antibiotic-resistant enterococci. Antimicrob. Agents Chemother. 63, e01454-19.
(24) Karas, B. J., Diner, R. E., Lefebvre, S. C., McQuaid, J., Phillips, A. P. R., Noddings, C. M., Brunson, J. K., Valas, R. E., Deerinck, T. J., Jablanovic, J., Gillard, J. T. F., Beeri, K., Ellisman, M. H., Glass, J. I., Hutchison, C. A., 3rd, Smith, H. O., Venter, J. C., Allen, A. E., Dupont, C. L., and Weyman, P. D. (2015) Designer diatom episomes delivered by bacterial conjugation. Nat. Commun. 6, 6925.
(25) Brumwell, S. L., Macleod, M. R., Huang, T., Cochrane, R., Meaney, R. S., Zamani, M., Matysiakiewicz, O., Dan, K. N., Janakirama, P., Edgell, D. R., Charles, T. C., Finan, T. M., and Karas, B. J. (2019) Designer Sinorhizobium meliloti strains and multi-functional vectors enable direct inter-kingdom DNA transfer. PLOS One. 14, e0206781.
(26) Hayman, G. T., and Bolen, P. L. (1993) Movement of shuttle plasmids from Escherichia coli into yeasts other than Saccharomyces cerevisiae using trans-kingdom conjugation. Plasmid. 30, 251-257.
(27) Moriguchi, K., Edahiro, N., Yamamoto, S., Tanaka, K., Kurata, N., and Suzuki, K. (2013) Transkingdom genetic transfer from Escherichia coli to Saccharomyces cerevisiae as a simple gene introduction tool. Appl. Environ. Microbiol. 79, 4393-4400.
(28) Soltysiak, M. P. M., Meaney, R. S., Hamadache, S., Janakirama, P., Edgell, D. R., and Karas, B. J. (2019) Trans-kingdom conjugation within solid media from Escherichia coli to Saccharomyces cerevisiae. Int. J. Mol. Sci. 20, 5212.
(29) Zoolkefli, F. I. R. M., Moriguchi, K., Cho, Y., Kiyokawa, K., Yamamoto, S., and Suzuki, K. (2021) Isolation and analysis of donor chromosomal genes whose deficiency is responsible for accelerating bacterial and trans-kingdom conjugations by IncP1 T4SS machinery. Front. Microbiol. 12, 971.
(30) Norberg, P., Bergström, M., Jethava, V., Dubhashi, D., and Hermansson, M. (2011) The IncP-1 plasmid backbone adapts to different host bacterial species and evolves through homologous recombination. Nat. Commun. 2, 268.
(31) Pansegrau, W., Lanka, E., Barth, P. T., Figurski, D. H., Guiney, D. G., Haas, D., Helinski, D. R., Schwab, H., Stanisich, V. A., and Thomas, C. M. (1994) Complete nucleotide sequence of Birmingham IncPa plasmids: compilation and comparative analysis. J. Mol. Biol. 239, 623-663.
(32) Ziegelin, G., Fürste, J. P., and Lanka, E. (1989) TraJ protein of plasmid RP4 binds to a 19-base pair invert sequence repetition within the transfer origin. J. Biol. Chem. 264, 11989-11994.
(33) Fürste, J. P., Pansegrau, W., Ziegelin, G., Kröger, M., and Lanka, E. (1989) Conjugative transfer of promiscuous IncP plasmids: interaction of plasmid-encoded products with the transfer origin. Proc. Natl. Acad. Sci. U.S.A 86, 1771-1775.
(34) Pansegrau, W., Balzer, D., Kruft, V., Lurz, R., and Lanka, E. (1990) In vitro assembly of relaxosomes at the transfer origin of plasmid RP4. Proc. Natl. Acad. Sci. U.S.A. 87, 6555-6559.
(35) Pansegrau, W., Schröder, W., and Lanka, E. (1993) Relaxase (TraI) of IncP alpha plasmid RP4 catalyzes a site-specific cleaving-joining reaction of single-stranded DNA. Proc. Natl. Acad. Sci. U.S.A 90, 2925-2929.
(36) Pansegrau, W., and Lanka, E. (1996) Mechanisms of initiation and termination reactions in conjugative DNA processing: independence of tight substrate binding and catalytic activity of relaxase (TraI) of IncPa plasmid RP4*. J. Biol. Chem. 271, 13068-13076.
(37) Ziegelin, G., Pansegrau, W., Lurz, R., and Lanka, E. (1992) TraK protein of conjugative plasmid RP4 forms a specialized nucleoprotein complex with the transfer origin. J. Biol. Chem. 267, 17279-17286.
(38) Rees, C. E., and Wilkins, B. M. (1990) Protein transfer into the recipient cell during bacterial conjugation: studies with F and RP4. Mol. Microbiol. 4, 1199-1205.
(39) Cabezón, E., Sastre, J. I., and de la Cruz, F. (1997) Genetic evidence of a coupling role for the TraG protein family in bacterial conjugation. Mol. Gen. Genet. 254, 400-406.
(40) Grahn, A. M., Haase, J., Bamford, D. H., and Lanka, E. (2000) Components of the RP4 conjugative transfer apparatus form an envelope structure bridging inner and outer membranes of donor cells: implications for related macromolecule transport systems. J. Bacteriol. 182, 1564-1574.
(41) Eisenbrandt, R., Kalkum, M., Lurz, R., and Lanka, E. (2000) Maturation of IncP pilin precursors resembles the catalytic dyad-like mechanism of leader peptidases. J. Bacteriol. 182, 6751-6761.
(42) Eisenbrandt, R., Kalkum, M., Lai, E. M., Lurz, R., Kado, C. I., and Lanka, E. (1999) Conjugative pili of IncP plasmids, and the Ti plasmid T pilus are composed of cyclic subunits. J. Biol. Chem. 274, 22548-22555.
(43) Gordon, Z. B., Soltysiak, M. P. M., Leichthammer, C., Therrien, J. A., Meaney, R. S., Lauzon, C., Adams, M., Lee, D. K., Janakirama, P., Lachance, M.-A., and Karas, B. J. (2019) Development of a transformation method for Metschnikowia borealis and other CUG-Serine Yeasts. Genes. 10, 78.
(44) Karas, B. J., Tagwerker, C., Yonemoto, I. T., Hutchison, C. A., 3rd, and Smith, H. O. (2012) Cloning the Acholeplasma laidlawii PG-8A genome in Saccharomyces cerevisiae as a yeast centromeric plasmid. ACS Synth. Biol. 1, 22-28.
(45) Marillonnet, S., and Grützner, R. (2020) Synthetic DNA assembly using golden gate cloning and the hierarchical modular cloning pipeline. Curr. Protoc. Mol. Biol. 130, e115.
(46) Engler, C., Kandzia, R., and Marillonnet, S. (2008) A one pot, one step, precision cloning method with high throughput capability. PLOS One. 3, e3647.
(47) Ng, R., and Abelson, J. (1980) Isolation and sequence of the gene for actin in Saccharomyces cerevisiae. Proc. Natl. Acad. Sci. U.S.A 77, 3912-3916.
(48) Ruotsalainen, P., Penttinen, R., Mattila, S., and Jalasvuori, M. (2019) Midbiotics: conjugative plasmids for genetic engineering of natural gut flora. Gut Microbes. 10, 643-653.
(49) Hullahalli, K., Rodrigues, M., and Palmer, K. L. (2017) Exploiting CRISPR-Cas to manipulate Enterococcus faecalis populations. Elife. 6, e26664.
(50) Ram, G., Ross, H. F., Novick, R. P., Rodriguez-Pagan, I., and Jiang, D. (2018) Conversion of staphylococcal pathogenicity islands to CRISPR-carrying antibacterial agents that cure infections in mice. Nat. Biotechnol. 36, 971-976.
(51) Lam, K. N., Spanogiannopoulos, P., Soto-Perez, P., Alexander, M., Nalley, M. J., Bisanz, J. E., Nayak, R. R., Weakley, A. M., Yu, F. B., and Turnbaugh, P. J. (2021) Phage-delivered CRISPR-Cas9 for strain-specific depletion and genomic deletions in the gut microbiome. Cell Rep. 37, 109930.
(52) Vercoe, R. B., Chang, J. T., Dy, R. L., Taylor, C., Gristwood, T., Clulow, J. S., Richter, C., Przybilski, R., Pitman, A. R., and Fineran, P. C. (2013) Cytotoxic chromosomal targeting by CRISPR/Cas systems can reshape bacterial genomes and expel or remodel pathogenicity islands. PLOS Genet. 9, e1003454.
(53) Kim, J.-S., Cho, D.-H., Park, M., Chung, W.-J., Shin, D., Ko, K. S., and Kweon, D.-H. (2016) CRISPR/Cas9-mediated re-sensitization of antibiotic-resistant Escherichia coli harboring extended-spectrum β-lactamases. J. Microbiol. Biotechnol. 26, 394-401.
(54) Wang, P., He, D., Li, B., Guo, Y., Wang, W., Luo, X., Zhao, X., and Wang, X. (2019) Eliminating mcr-1-harbouring plasmids in clinical isolates using the CRISPR/Cas9 system. J. Antimicrob. Chemother. 74, 2559-2565.
(55) Valderrama, J. A., Andrés Valderrama, J., Kulkarni, S. S., Nizet, V., and Bier, E. (2019) A bacterial gene-drive system efficiently edits and inactivates a high copy number antibiotic resistance locus. Nature Communications. 10, 1-8.
(56) Wang, K., and Nicholaou, M. (2017) Suppression of antimicrobial resistance in MRSA using CRISPR-dCas9. Clin. Lab. Sci. 30, 207-213.
(57) Li, Q., Zhao, P., Li, L., Zhao, H., Shi, L., and Tian, P. (2020) Engineering a CRISPR interference system to repress a class 1 integron in Escherichia coli. Antimicrob. Agents Chemother. 64, e01789-19.
(58) Park, J. Y., Moon, B. Y., Park, J. W., Thornton, J. A., Park, Y. H., and Seo, K. S. (2017) Genetic engineering of a temperate phage-based delivery system for CRISPR/Cas9 antimicrobials against Staphylococcus aureus. Sci. Rep. 7, 44929.
(59) Gietz, R. D., and Woods, R. A. (2001) Genetic transformation of yeast. Biotechniques. 30, 816-20, 822-6, 828 passim.
(60) Martín, J. F. (2015) Fungal transformation: from protoplasts to targeted recombination systems, in Genetic Transformation Systems in Fungi, Volume 1 (van den Berg, M. A., and Maruthachalam, K., Eds.), pp 3-18. Springer International Publishing, Cham.
(61) Zatyka, M., Jagura-Burdzy, G., and Thomas, C. M. (1994) Regulation of transfer genes of promiscuous IncP alpha plasmid RK2: repression of Tra1 region transcription both by relaxosome proteins and by the Tra2 regulator TrbA. Microbiology. 140 (Pt 11), 2981-2990.
(62) Kostylev, M., Otwell, A. E., Richardson, R. E., and Suzuki, Y. (2015) Cloning should be simple: Escherichia coli DH5α-mediated assembly of multiple DNA fragments with short end homologies. PLOS One. 10, e0137466.
(63) Stinchcomb, D. T., Struhl, K., and Davis, R. W. (1979) Isolation and characterisation of a yeast chromosomal replicator. Nature. 282, 39-43.
(64) Clarke, L., and Carbon, J. (1980) Isolation of a yeast centromere and construction of functional small circular chromosomes. Nature. 287, 504-509.
(65) Cai, P., Gao, J., and Zhou, Y. (2019) CRISPR-mediated genome editing in non-conventional yeasts for biotechnological applications. Microb. Cell Fact. 18, 63.
(66) Morio, F., Lombardi, L., and Butler, G. (2020) The CRISPR toolbox in medical mycology: state of the art and perspectives. PLOS Pathog. 16, e1008201.
(67) Raschmanová, H., Weninger, A., Glieder, A., Kovar, K., and Vogl, T. (2018) Implementing CRISPR-Cas technologies in conventional and non-conventional yeasts: current state and future prospects. Biotechnol. Adv. 36, 641-665.
(68) Stovicek, V., Holkenbrink, C., and Borodina, I. (2017) CRISPR/Cas system for yeast genome engineering: advances and applications. FEMS Yeast Res. 17.
(69) Shan, L., Dai, Z., and Wang, Q. (2021) Advances and opportunities of CRISPR/Cas technology in bioengineering non-conventional yeasts. Front Bioeng. Biotechnol. 9, 765396.
(70) Uthayakumar, D., Sharma, J., Wensing, L., and Shapiro, R. S. (2021) CRISPR-based genetic manipulation of Candida species: historical perspectives and current approaches. Frontiers in Genome Editing. 2, 606281.
(71) Vyas, V. K., Barrasa, M. I., and Fink, G. R. (2015) A Candida albicans CRISPR system permits genetic engineering of essential genes and gene families. Sci. Adv. 1, e1500248.
(72) Enkler, L., Richer, D., Marchand, A. L., Ferrandon, D., and Jossinet, F. (2016) Genome engineering in the yeast pathogen Candida glabrata using the CRISPR-Cas9 system. Sci. Rep. 6, 35766.
(73) Rosiana, S., Zhang, L., Kim, G. H., Revtovich, A. V., Uthayakumar, D., Sukumaran, A., Geddes-McAlister, J., Kirienko, N. V., and Shapiro, R. S. (2021) Comprehensive genetic analysis of adhesin proteins and their role in virulence of Candida albicans. Genetics. 217, iyab003.
(74) Shapiro, R. S., Chavez, A., Porter, C. B. M., Hamblin, M., Kaas, C. S., DiCarlo, J. E., Zeng, G., Xu, X., Revtovich, A. V., Kirienko, N. V., Wang, Y., Church, G. M., and Collins, J. J. (2018) A CRISPR-Cas9-based gene drive platform for genetic interaction analysis in Candida albicans. Nat. Microbiol. 3, 73-82.
(75) Zoppo, M., Luca, M. D., Villarreal, S. N., Poma, N., Barrasa, M. I., Bottai, D., Vyas, V. K., and Tavanti, A. (2019) A CRISPR/Cas9-based strategy to simultaneously inactivate the entire ALS gene family in Candida orthopsilosis. Future Microbiol. 14, 1383-1396.
(76) Santana, D. J., and O'Meara, T. R. (2021) Forward and reverse genetic dissection of morphogenesis identifies filament-competent Candida auris strains. Nature Communications. 12, 1-13.
(77) Huang, M. Y., Woolford, C. A., May, G., McManus, C. J., and Mitchell, A. P. (2019) Circuit diversification in a biofilm regulatory network. PLOS Pathog. 15, e1007787.
(78) Min, K., Biermann, A., Hogan, D. A., and Konopka, J. B. (2018) Genetic analysis of NDT80 family transcription factors in Candida albicans using new CRISPR-Cas9 approaches. mSphere. 3, e00545-18.
(79) Wensing, L., Sharma, J., Uthayakumar, D., Proteau, Y., Chavez, A., and Shapiro, R. S. (2019) A CRISPR interference platform for efficient genetic repression in Candida albicans. mSphere. 4, e00002-19.
(80) Ennis, C. L., Hernday, A. D., and Nobile, C. J. (2021) A markerless CRISPR-mediated system for genome editing in Candida auris reveals a conserved role for Cas5 in the caspofungin response. Microbiol. Spectr. 9, e0182021.

1. A DNA fragment for facilitating the transfer of a plasmid transferable (“transferable plasmid”) by conjugation from a donor bacterium to a recipient eukaryotic cell, the DNA fragment comprising a mutated traJ promoter operatively linked to a traJ gene, wherein the mutated traJ promoter includes a mutation that enables transfer of the transferable plasmid by conjugation from the donor bacterium to the recipient eukaryotic cell.

2-6. (canceled)

7. The DNA fragment of claim 1, wherein the DNA fragment is included in the transferable plasmid and wherein the DNA fragment further comprises a gene of interest that can be expressed in the recipient eukaryotic cell.

8. (canceled)

9. The DNA fragment of claim 7, wherein the gene of interest encodes for a protein that kills the recipient eukaryotic cell.

10. The DNA fragment of claim 7, wherein expression of the gene of interest is inducible.

11. (canceled)

12. The DNA fragment of claim 1, wherein the recipient eukaryotic cell the fungal cell is a yeast cell.

13-19. (canceled)

20. The DNA fragment of claim 1, wherein the traJ promoter is the traJ promoter of a pTA-Mob 2.0 plasmid.

21. The DNA fragment of claim 1, wherein the mutated traJ promoter includes the nucleotide sequence ccgag (SEQ ID NO: 3).

22. The DNA fragment of claim 1, wherein the mutated traJ promoter comprises SEQ ID NO: 2.

23. The DNA fragment of claim 1, wherein at least one of (i) the DNA fragment, (ii) the donor bacterium, (iii) the transferable plasmid and (iv) a non-transferable plasmid within the donor bacterium, includes further mutations that in combination with the mutated traJ promoter improves the transfer of the transferable plasmid from the donor bacterium to the recipient cell of the eukaryotic species.

24. A bacterium for facilitating the transfer of a plasmid transferable (“transferable plasmid”) by conjugation from the bacterium to a recipient eukaryotic cell, the bacterium comprising: (i) a transferable plasmid and (ii) a DNA fragment carrying a mutated traJ promoter operatively linked to a traJ gene, wherein the mutated traJ promoter includes a mutation that enables transfer of the transferable plasmid by conjugation from the donor bacterium to the recipient eukaryotic cell.

25-29. (canceled)

30. The bacterium of claim 24, wherein the DNA fragment is included in the transferable plasmid and wherein the DNA fragment further carries a gene of interest and the gene of interest is expressed in the recipient eukaryotic cell.

31. (canceled)

32. The bacterium of claim 30, wherein the gene of interest encodes for a protein that kills the recipient eukaryotic cell.

33. The bacterium of claim 30, wherein expression of the gene of interest is inducible.

34. (canceled)

35. The bacterium of claim 24 wherein the recipient eukaryotic cell is a yeast cell.

36-42. (canceled)

43. The bacterium of claim 24, wherein the traJ promoter is of a pTA-Mob 2.0 plasmid.

44. The bacterium of claim 24, wherein the mutated traJ promoter includes the nucleotide sequence ccgag (SEQ ID NO: 3).

45. The bacterium of claim 24, wherein the mutated traJ promoter comprises SEQ ID NO: 2.

46. The bacterium of claim 24, wherein at least one of the DNA fragment, the bacterium and the transferable plasmid, includes further mutations that in combination with the mutated traJ improves the transfer of the transferable plasmid from the donor bacterium to the recipient eukaryotic cell.

47. A method of conjugatively transferring a gene of interest from a donor bacterium into a recipient eukaryotic cell, the method comprising:
(a) providing the bacterium of claim 30, wherein the gene of interest is not native to the donor bacterium, and
(b) conjugatively transferring the transferable plasmid from the bacterium to the recipient eukaryotic cell, thereby transferring the gene of interest to the recipient eukaryotic cell.

48-49. (canceled)

50. The method of claim 47, wherein the gene of interest encodes a protein that kills the recipient eukaryotic cell.

51. (canceled)

52. The method of claim 47, wherein
expression of the gene of interest is inducible.

53. The method of claim 47, wherein the mutated traJ promoter includes the nucleotide sequence ccgag (SEQ ID NO: 3).

54. The method of claim 47, wherein the mutated traJ promoter comprises SEQ ID NO: 2.
